(19) United States
(12) Patent Application Publication
(10) Pub. No.: US 2012/0231083 A1
(43) Pub. Date: Sep. 13, 2012

(54) SUSTAINED RELEASE CANNABINOID MEDICAMENTS

(75) Inventors: David Carley, Evanston, IL (US); Kenton Fedde, Wildwood, MO (US); John KOLENG, Austin, TX (US); Peter LETENDRE, Lafayette, CO (US)

(73) Assignees: The Board of Trustees of the University of Illinois, Urbana, IL (US); PIER PHARMACEUTICALS, Louisville, CO (US)

(21) Appl. No.: 13/474,666

(22) Filed: May 17, 2012

Related U.S. Application Data
(63) Continuation of application No. PCT/US10/57302, filed on Nov. 18, 2010.

(51) Int. Cl. A61K 9/14; A61P 11/00; A61K 31/352

(52) U.S. Cl. 424/494; 514/454; 424/490; 424/497

(57) ABSTRACT

The present invention provides a medicament which results in delivery of a therapeutic level of one or more cannabinoids during a clinically relevant therapeutic window. The therapeutic window is a longer window than provided by an immediate release medicament such as Marinol containing an equivalent amount of the cannabinoid. Oral administration of the present compositions provides therapeutic dosing while maintaining safe, side effect sparing, levels of a cannabinoid. The present invention also provides methods of treating cannabinoid-sensitive disorders.

### CROSS REFERENCE TO RELATED APPLICATIONS

This application is a continuation of PCT/US2010/057302, filed 10 Nov. 2010, which claims priority to U.S. Provisional 61/262,523, filed 18 Nov. 2009, which are hereby incorporated by reference in their entirety.

### TECHNICAL FIELD

The present invention relates to cannabinoid compositions and methods of treating cannabinoid-sensitive disorders (e.g. apnea) with cannabinoids.

### BACKGROUND

Over the past several years, much effort has been devoted to the study of a discrete group of breathing disorders that occur primarily during sleep with consequences that may persist throughout the waking hours, most commonly in the form of sleepiness and/or cognitive/motor impairment, thereby manifesting itself into substantial economic loss (e.g., thousands of lost man-hours) or employment safety factors (e.g., employee non-attentiveness during operation of heavy-machinery). Sleep-related breathing disorders are characterized by repetitive reduction in breathing (hypopnea), cessation of breathing (apnea), or a continuous or sustained reduction in ventilation (hypoventilation).

In general, sleep apnea is defined as an intermittent cessation of airflow at the nose and mouth during sleep. By convention, apneas of at least 10 seconds in duration have been considered important, but in most individuals, the apneas are 20-30 seconds in duration and may be as long as 2-3 minutes. While there is some uncertainty as to the minimum number of apneas that should be considered clinically important, by the time most individuals come to attention of the medical community they have at least 10 to 15 events per hour of sleep.

Sleep apneas have been classified into three types: central, obstructive, and mixed. In central sleep apnea the neural drive to all respiratory muscles is transiently abolished. In obstructive sleep apneas, airflow ceases despite continuing respiratory drive because of occlusion of the oropharyngeal airway. Mixed apneas, which comprise a central apnea followed by an obstructive component, are a variant of obstructive sleep apnea. The most common type of apnea is obstructive sleep apnea. Although airflow persists during hypopneas, like apneas they are associated with reduced oxygen levels in the arterial blood and/or arousals from sleep. Apneas and hypopneas are viewed as carrying equal clinical significance. Because airflow persists, hypopneas are not classified as either central or obstructive.

Obstructive sleep apnea syndrome (OSAS) has been identified in as many as 24% of working adult men and 9% of similar women, with peak prevalence in the sixth decade of life. Habitual heavy snoring, which is an almost invariant feature of OSAS, has been described in up to 24% of middle aged men, and 14% of similarly aged women, with even greater prevalence in older subjects.

Obstructive sleep apnea syndrome's definitive event is the occlusion of the upper airway, frequently at the level of the oropharynx. The resultant apnea generally leads to a progressive-type asphyxia until the individual is briefly aroused from the sleeping state, thereby restoring airway patency and thus restoring airflow.

An important factor that leads to the collapse of the upper airway in OSAS is the generation of a critical subatmospheric pressure during the act of inspiration that exceeds the ability of the airway dilator and abductor muscles to maintain airway stability. Sleep plays a crucial role by reducing the activity of the muscles of the upper airways including the dilator and abductor muscles.

In most individuals with OSAS the patency of the airway is also compromised structurally and is therefore predisposed to occlusion. In a minority of individuals the structural compromise is usually due to obvious anatomic abnormalities, i.e, adenotonsillar hypertrophy, retrognathia, or macroglossia. However, in the majority of individuals predisposed to OSAS, the structural abnormality is simply a subtle reduction in airway size, i.e., “pharyngeal crowding.” Obesity also frequently contributes to the reduction in size seen in the upper airways. The act of snoring, which is actually a high-frequency vibration of the palatal and pharyngeal soft tissues, usually aggravates the narrowing via the production of edema in the soft tissues.

The recurrent episodes of nocturnal asphyxia and of arousal from sleep that characterize OSAS lead to a series of secondary physiologic events, which in turn give rise to the clinical complications of the syndrome. The most common manifestations are neuropsychiatric and behavioral disturbances that are thought to arise from the fragmentation of sleep and loss of slow-wave sleep induced by the recurrent arousal responses. Nocturnal cerebral hypoxia also may play an important role. The most pervasive manifestation is excessive daytime sleepiness, although insomnia is common in the elderly with OSAS. OSAS is now recognized as a leading cause of daytime sleepiness and has been implicated as an important risk factor for such problems as motor vehicle accidents. Other related symptoms include intellectual impairment, memory loss, personality disturbances, and impotence.

Other major manifestations are cardiorespiratory in nature and are thought to arise from the recurrent episodes of nocturnal asphyxia. Most individuals demonstrate a cyclical slowing of the heart during the apneas to 30 to 50 beats per minute, followed by tachycardia of 90 to 120 beats per minute during the ventilatory phase. A small number of individuals develop severe bradycardia with asystoles of 8 to 12 seconds in duration or dangerous tachyarrhythmias, including unsustained ventricular tachycardia. OSAS also aggravates left ventricular failure in patients with underlying heart disease. This complication is most likely due to the combined effects of increased left ventricular afterload during each obstructive event, secondary to increased negative intrathoracic pressure, recurrent nocturnal hypoxemia, and chronically elevated sympathoadrenal activity.

Central sleep apnea is less prevalent as a syndrome than OSAS, but can be identified in a wide spectrum of patients with medical, neurological, and/or neuromuscular disorders associated with diurnal alveolar hypoventilation or periodic breathing. The definitive event in central sleep apnea is transient abolition of central drive to the ventilatory muscles. The resulting apnea leads to a primary sequence of events similar to those of OSAS. Several underlying mechanisms can result in cessation of respiratory drive during sleep. First are defects in the metabolic respiratory control system and respiratory neuromuscular apparatus. Other central sleep apnea disorders arise from transient instabilities in an otherwise intact respiratory control system.

Many healthy individuals demonstrate a small number of central apneas during sleep, particularly at sleep onset and in REM sleep. These apneas are not associated with any physiological or clinical disturbance. In individuals with clinically significant central sleep apnea, the primary sequence of events that characterize the disorder leads to prominent physiological and clinical consequences. In those individuals with central sleep apnea alveolar hypoventilation syndrome, daytime hypercapnia and hypoxemia are usually evident and the clinical picture is dominated by a history of recurrent respiratory failure, polycythemia, pulmonary hypertension, and right-sided heart failure. Complaints of sleeping poorly, morning headache, and daytime fatigue and sleepiness are also prominent. In contrast, in individuals whose central sleep apnea results from instability in respiratory drive, the clinical picture is dominated by features related to sleep disturbance, including recurrent nocturnal awakenings, morning fatigue, and daytime sleepiness.

Currently, the most common and most effective treatment, for adults with sleep apnea and other sleep-related breathing disorders, are mechanical forms of therapy that deliver positive airway pressure (PAP). Under PAP treatment, an individual wears a tight-fitting plastic mask over the nose when sleeping. The mask is attached to a compressor, which forces air into the nose creating a positive pressure within the patient's airways. The principle of the method is that pressurizing the airways provides a mechanical “splinting” action, which prevents airway collapse and therefore, obstructive sleep apnea. Although an effective therapeutic response is observed in most patients who undergo PAP treatment, many patients cannot tolerate the apparatus or pressure and refuse treatment. Moreover, covert monitoring studies clearly demonstrate that long-term compliance with PAP treatment is very poor. A variety of upper airway and craniofacial surgical procedures have been attempted for treatment of OSAS. Adenotonsillectomy appears to be an effective cure for OSAS in many children, but upper airway surgery is rarely curative in adult patients with OSAS. Surgical “success” is generally taken to be a 50% reduction in apnea incidence and there are no useful screening methods to identify the individuals that would benefit from the surgery versus those who would not derive a benefit.

Recently, the present inventors demonstrated that intraperitoneal injection of THC at 10 mg/kg in a rat model of sleep apnea reduced breathing cessation during non-REM (“NREM”) sleep as described in US 2004/0127572. What is needed in the art is a convenient oral medicament in an amount that reduces apnea.

Also needed is an oral dosage that is effective yet minimizes undesirable effects (e.g. of inducing psychotropic responses). Also needed is a medicament that provides therapeutic efficacy for a period of time roughly equivalent to a typical human sleep period (e.g. about 6 to 8 hours) and that doesn't require repeated dosage through that period. Also needed is an oral medicament that allows the subject to wake from sleep without residual side effects that negatively impact wakefulness and alertness without other known affects such as an overly-stimulated appetite.

### SUMMARY OF THE INVENTION

The present invention provides new compositions (medicaments) that result in delivery of a therapeutic level of one or more cannabinoids during a clinically relevant therapeutic window. The therapeutic window provided by present medicaments is a longer window than provided by immediate release medicaments such as Marinol, containing an equivalent amount of the cannabinoid. The present compositions are especially suitable for conditions that are benefitted by therapeutic levels for more than about 5 hours, e.g. about 6 to about 8 hours or for as long as about 8 or about 12 hours.

The present compositions provide therapeutic dosing at levels controlled to generally avoid levels typically associated with certain side effects. The present compositions comprise a cannabinoid partitioned in an immediate release compartment and in a delayed release compartment.

The present invention provides methods of treating cannabinoid-sensitive disorders. Examples of cannabinoid-sensitive disorders are sleep apnea, anxiety, stress, headache, nausea, glaucoma, pain, arthritis, irritable bowel syndrome, ulcerative colitis, Crohn's disease, anorexia or cachexia syndrome, bladder dysfunction, spasticity due to multiple sclerosis, Huntington's disease, and Alzheimer's disease.

Optionally, the present medicaments provide oral dosing of about 0.1 to about 75 mg of a cannabinoid. Optionally, the subject sleeps during the therapeutic window.

Optionally, the oral medicaments of the present invention provide a therapeutic response without causing, or while causing only mild, side effects associated with cannabinoids. Optionally, the oral medicaments of the present invention, when administered to a subject immediately before a sleep cycle, provide a therapeutic response without causing (or without substantially causing) side effects once the subject has awoken (e.g. post treatment window).

In one embodiment, the invention provides a medicament comprising a cannabinoid dose, an excipient, and one or more release modifiers present in an amount to provide, when administered orally, a therapeutic window which extends over both an early treatment window (e.g. providing a Tmax of about 30 minutes to about 3 hours) and a late therapeutic window.

The applicants also describe an invention comprising treating a subject with a low dose (e.g. about 0.1 to about 20 mg) of a cannabinoid (e.g. THC) in an oral dosage form that does not contain a release modifier (e.g. Marinol).

### BRIEF DESCRIPTION OF THE DRAWINGS

FIG. 1 depicts the % of subjects with a 75% reduction in AHI versus duration of reduction: the effect of THC dose.

FIG. 2 depicts dose and time dependent effects of THC on apnea suppression.

FIG. 3 depicts AHI in sleep apnea patients during a target treatment window.

FIG. 4 depicts the relationship of Cmax (ng/ml) and cannabinoid amount (mg) for an immediate release compartment (Marinol formulation).

FIG. 5 depicts plasma profiles depicted in US 2009/0181080 of a THC formulation.

### DETAILED DESCRIPTION OF THE INVENTION

As used here, the following definitions and abbreviations apply.
“AHI” means apnea-hypopnea index, which is calculated by dividing the number of apnea and hypopnea events by the number of hours of sleep. The AHI index generally quantifies the overall severity of sleep apnea including sleep disruptions and desaturations. Typically, an AHI of 5-15 is considered mild, 15-30 is moderate, and above 30 is severe.
“AUC” (area-under-the-curve) is the overall amount of THC (or metabolite thereof) in the bloodstream or plasma after a dose. AUC can be calculated by collecting multiple blood samples over a period of time, graphing the drug concentrations, and calculating the drug as the area under this drug concentration curve. AUC can be expressed in units of amount of THC×time/volume (e.g. ng×hr/ml).
“Cmax” means the maximum plasma concentration of THC (or a metabolite thereof) during an interval of time.
“Cannabinoid-sensitive disorder” means a disorder that, when a cannabinoid or a cannabinoid receptor modulator is administered, modulates a pathophysiologic pathway that ameliorates the disorder or clinically relevant symptoms thereof. Relevant pathophysiologic pathways can be desirably modulated by present medicaments. For example, administration may modulate the pathways of acid (e.g. GABA, glutamate), monoamine (e.g. histamine, dopamine, serotonin, noradrenaline) purine (e.g. adenosine, ADP, ATP), peptide (e.g. somatostatin, neuropeptide Y, neurokinin, cholecystokinin), vanilloid, prostanoid, opiod and/or other neurotransmitters. Accordingly, cannabinoid-sensitive disorders include disorders mediated by or sensitive to neurotransmitter action.
“Cₘᵢₙ” (or trough) is the lowest concentration of THC (or a metabolite thereof) in the plasma (following the Cmax) within a defined treatment window.
“Delayed release” (or “DR”) means release of a cannabinoid in a manner such that release of the cannabinoid in vivo is delayed in comparison to the release from an immediate release compartment (e.g. a Marinol formulation). In vivo release can be estimated from an in vitro release assay appropriate for the formulation (e.g. as set forth in Example 10). Examples of medicament-types which display delayed release include extended release, sustained release, continuous release, timed-release, and pulsatile release (e.g. timed release or instant release plus timed release).
“Delayed release dosage compartment” is a dosage compartment comprising a release-modifying amount of a release modifier. In one embodiment, the release of a cannabinoid partitioned in a delayed release compartment is delayed by about any of: 100%, 150%, 200%, 300%, 400%, 500%, 600%, 700%, or 800%, compared to an immediate release medicament such as Marinol in an in vitro dissolution assay (e.g. Example 10).
“Dosage compartment” means a discrete layer, sphere, fraction or formulation encompassing a cannabinoid. In one embodiment, the delayed release compartment may be dispersed in or co-localized to a component of the immediate release compartment. It should further be understood that in some dosage forms, the immediate release dosage compartment(s) and the delayed release dosage compartment(s) are distinguished by their location within the dosage form. By way of example, in the monolithic matrix tablets of Example 17—e.g. a matrix, the immediate release compartment is the outside portion of the tablet (i.e. the release of the drug in this portion is not delayed by a release modifier). Similarly, in this example, the delayed release compartment is the portion of the tablet towards the interior, where the release modification (or release modifier) is the functional result of the location within the dosage form.
“Examplary” (or “e.g.” or “by example”) means a non-limiting example.
“Extended therapeutic window” means a therapeutic window which extends over both an early treatment window and a late treatment window 0039 “Immediate release dosage compartment” (or “IR”) means a dosage compartment that does not contain a release modifier in a release modifying amount. In one embodiment, the immediate release dosage compartment is the portion of a dosage form that releases greater than 75% of the cannabinoid contained in the portion within 60 minutes, or greater than 50% within 30 minutes in an in vitro dissolution assay (e.g. a dissolution assay described in Example 10). Typically, an immediate release compartment releases the cannabinoid in an in vitro dissolution assay at a rate of at least 50% of a Marinol formulation, e.g. about 50% to 150% of a Marinol formulation.
“Marinol” means a gel capsule medicament of dronabinol as it generally is formulated and available under the trademark MARINOL®. Where reference is made to Marinol at a concentration that is not commercially available, it is meant to refer to a medicament formulated similarly to other strengths of MARINOL®, i.e. containing dronabinol, gelatin, glycerin, and sesame oil.
“Release modifiers” means a pharmaceutically acceptable ingredient or ingredients that act independently or in concert to cause the delayed release of a cannabionoid from a medicament or dosage compartment and/or that slow or delay the absorption of the cannabionoid in the gut.
“Substantially similar”, as it relates to a referenced quantifiable parameter (e.g. THC plasma level, Cmax, Tmax, or therapeutic response) means that the subject parameter is from about 50% to about 200% of the referenced parameter, or from about 75% to about 150%, or about 80% to about 120%.
“Therapeutic window” means a period of time during which a therapeutically effective level of drug is maintained.
“Treatment window” means the period of time beginning at the time of administration (i.e. Tₒ) of the drug composition and ending at a defined time. The treatment window can be further divided into sub-periods, such as “early treatment window” (e.g. T₁ₕᵣ−T₄ₕᵣ or T₁ₕᵣ−T₅ₕᵣ) or “late treatment window” (e.g. T₄ₕᵣ−T₈ₕᵣ or T₃ₕᵣ−T₈ₕᵣ). The units of subscript of “T”, where not stated, are “hours” (for example, T₁=T₁ₕᵣ).

“THC” means a cannabinoid of the present invention (as described below).
“Therapeutic efficiency” means the ratio of therapeutic response to side effects (i.e. any treatment-related effects that are not a therapeutic response). 0047 “Therapeutic response” means any response that can be considered to represent a reduction in the signs or symptoms of a medical condition. For apnea, a therapeutic response is, for example, a reduction in apnea-hypopnea index, snoring, oxygen desaturation of the arterial blood, or sleep disruption.
“Tmax” means the time between the administration of the medicament and the time that a maximum plasma level (Cmax) of the referenced cannabinoid (or metabolite) is achieved.

Present Medicaments for Cannabinoid Sensitive Disorders

The present medicaments are surprisingly effective for treating certain cannabinoid sensitive disorders. Technical features include providing, when administered to certain subjects: (1) a therapeutic window which begins within about 30 minutes or about 1 hour or about 2 hours of administration (e.g. as shown by Example 3); (2) a therapeutic window that is about 1 to about 10 hours longer than the therapeutic window of an immediate release dosage (e.g. as shown in Example 5); and (3) plasma levels that do not elevate into a level where reduced therapeutic efficacy and/or deleterious side effects are produced (e.g. as shown in Example 3, it has been surprisingly discovered that certain patients with cannabinoid-sensitive disorders exhibit a non-monotonic dose-response of the inverted U type).

Cannabinoids

The compositions of the present invention provide one or more cannabinoids in a medicament that can deliver to a subject a desired target PK profile, where the PK profile achieves a therapeutic level of a cannabinoid during a therapeutic window. Cannabinoids of the present invention are any member of a group of substances that are structurally related to tetrahydrocannabinol and that bind to a cannabinoid receptor such as CB1 or CB2 or both (‘THC’). The cannabinoid can be a naturally occurring compound (e.g. present in Cannabis), a compound metabolized by a plant or animal, or a synthetic derivative.

The cannabinoid may be included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms.

The cannabinoids of the present invention are further meant to encompass natural cannabinoids, natural cannabinoids that have been purified or modified, and synthetically derived cannabinoids, for example, United States Patent Application Publication 2005/0266108, hereby incorporated by reference in its entirety, describes a method of purifying cannabinoids obtained from plant material.

The cannabinoids of the present invention can be any of 9-tetrahydrocannabinol, 8-tetrahydrocannabinol, (+)-1,1-dimethylheptyl analog of 7-hydroxy-delta-6-tetrahydrocannabinol, 3-(5′-cyano-1′,1′-dimethylpentyl)-1-(4-N-morpholinobutyryloxy) delta 8-tetrahydrocannabinol hydrochloride], dexanabinol, nabilone, levonantradol, or N-(2-hydroxyethyl)hexadecanoamide. The cannabinoids of the present invention can be any of the non-psychotropic cannabinoid 3-dimethylnepty 11 carboxylic acid homologine 8, delta-8-tetrahydrocannabinol. (J. Med. Chem. 35, 3135, 1992).

The cannabinoids of the present invention can further be any of the active metabolites, derivatives, or analogs as taught in the National Institute on Drug Abuse Research Monograph Series 79, “Structure-Activity Relationships of the Cannabinoids.

In certain embodiments of the present invention, the cannabinoid can be Delta-9-tetrahydrocannabinol, also known as dronabinol. Dronabinol is naturally-occurring and has been extracted from Cannabis sativa L. (marijuana). It has also been produced chemically as described in U.S. Pat. No. 3,668,224. Dronabinol is a light-yellow resinous oil that is sticky at room temperature, but hardens upon refrigeration. It turns to a flowable liquid when heated at higher temperatures. Dronabinol is insoluble in water and typically formulated in sesame oil. It has a pKa of 10.6 and an octanol-water partition coefficient: 6,000:1 at pH 7. Dronabinol is available in natural (extracted from plant) and synthetic forms. On the other hand, synthetic dronabinol may be utilized and may be synthesized using the starting materials: Olivetol and p-2,8-menthadien-2-ol (PMD).

The term “dronabinol” is further meant to encompass naturally occurring dronabinol, synthetically derived dronabinol, and synthetically modified dronabinol starting with a molecule obtained from a natural source for example, United States Patent Application Publication 2005/0171361, hereby incorporated by reference in its entirety, describes a method of extracting delta-9−THC acid from the plant material by chromatography and then synthetically converting it to dronabinol.

Structural features of the present cannabinoids, and structure-function relationships are well-known in the art and taught, for example, by Rao Rapaka and Alexandros Makriyannis in “Structure-Activity Relationships of the Cannabinoids”, NIDA Research Monograph 79 (1987).

PK Profile

Compositions of the present invention comprise cannabinoids in an amount of about 0.1 mg to about 75 mg.

These doses in the compositions of the present invention are formulated to provide PK profiles that unexpectedly result in therapeutically effective levels of a cannabinoid during a desired therapeutic window. Among the useful PK profiles of the present invention are the following (where the medicament of the present invention is administered at T₀):

PK Profile 1: A present THC medicament is orally administered resulting in (a) a THC plasma concentration of 50% to 200% from T₁ through T₄ when compared to the THC plasma concentration from T₁ through T₄ attained by orally administering a comparator of ½ of the THC dose in a Marinol capsule at Tₒ and (b) a THC plasma concentration of 50% to 200% from T₅ through T₈ when compared to the plasma concentration from T₅ through T₈ attained by orally administering a comparator of twice the THC dose in a Marinol capsule at T₀.

PK Profile 2: A present THC medicament is orally administered resulting in a THC plasma concentration of 50% to 200% from T₁ through T₇ when compared to the plasma concentration attained by orally administering a comparator of ½ of the THC dose in a Marinol capsule at Tₒ and ½ of the THC dose in a Marinol capsule at T₃.

PK Profile 3: A present THC medicament is orally administered resulting in a THC plasma concentration greater than about 150% from T₄ through T₆, when compared to the plasma concentration attained by orally administering ½ of the THC dose amount in a Marinol capsule. at T₄.

PK Profile 4: A present THC medicament is orally administered resulting in a THC plasma concentration at T₄ that is greater than about 75% when compared to the plasma level at T₃ attained by orally administering ½ of the THC dose in a Marinol capsule.

PK Profile 5: A present THC medicament is orally administered resulting in a THC AUC of 8.64-11.52 ng×hr/ml, a Cmax between 0.4 ng/ml and 4.5 ng/ml; and a Tmax between 0.5 and 3.5 hours, optionally between 0.5 and 1.5 hours.

PK Profile 6: A present THC medicament is orally administered resulting in a THC plasma concentration during an early-treatment window (e.g. during T₀ₕᵣ−T₄ₕᵣ) and a maximum THC plasma concentration (Cmax) that is substantially similar to that provided by an immediate release form (e.g. a single Marinol capsule containing about ½ the dose).

PK Profile 7: A present THC medicament is orally administered resulting in a THC Tmax substantially similar to the Tmax of an orally administered immediate release Marinol capsule containing about ½ the dose. Optionally, the PK profile provides a Tmax during an early treatment window (e.g. from T_{1/2hr}−T_{1.5hr}, T_{1/2hr}−T₁ₕᵣ or at about T₁ₕᵣ).

PK Profile 8: A present THC medicament is orally administered resulting in a plasma cannabinoid level (or therapeutic response) during a late-treatment window (e.g. during T₄ₕᵣ−T₈ₕᵣ) substantially similar to that produced when two Marinol capsules containing about ½ the dose are orally administered in succession (e.g. one at T₀ and one at about T₃ₕᵣ).

PK Profile 9: A present THC medicament is orally administered resulting in a THC plasma level during a late-treatment window (e.g. during T₄ₕᵣ−T₈ₕᵣ) substantially similar to that produced during an early-treatment window (e.g. during T₁ₕᵣ−T₄ₕᵣ) by a Marinol capsule containing about ½ the dose. Optionally, the plasma level and/or AUC during the late-treatment window is substantially similar to that produced during the early-treatment window by the Marinol capsule.

PK Profile 10: A present THC medicament is orally administered resulting in a plasma THC level during a late-treatment window (e.g. during T₄ₕᵣ−T₈ₕᵣ such as during T₄ₕᵣ−T₅ₕᵣ, T₅ₕᵣ−T₆ₕᵣ, and/or T₆ₕᵣ−T₇ₕᵣ) that is greater than that produced during a mid-treatment window (e.g. during T₃ₕᵣ−T₆ₕᵣ such as hours T₄ₕᵣ−T₅ₕᵣ and/or T₅ₕᵣ−T₆ₕᵣ) by a Marinol capsule containing about ½ the dose. Optionally, the plasma level and/or AUC during the late-treatment window is greater than that produced during the mid-treatment window by the Marinol capsule.

PK Profile 11: A present THC medicament is orally administered resulting in an AUC during a treatment window (e.g. during T₀ₕᵣ−T₈ₕᵣ) which is about 3-4 times greater than that of a Marinol capsule containing about ½ the dose. Optionally, the Cmax is substantially similar to that of the Marinol capsule. Optionally, the Tmax is less than about T₂ₕ (e.g. from about T_{0.5h}−T_{1.5h}, such as about T₁ₕ).

PK Profile 12: A present THC medicament is orally administered resulting in plasma levels and/or an AUC during an early treatment window (e.g. during T₀ₕᵣ−T₄ₕᵣ) which are substantially similar to (e.g. 50%-200% of) those produced when a reference Marinol capsule containing ½ the THC dose is orally administered.

PK Profile 13: The PK profile of PK Profile 12, where the plasma levels and/or an AUC during a late treatment window (e.g. between T₄ₕᵣ and T₈ₕᵣ or T₄ₕᵣ and T₆ₕᵣ) are greater than (e.g. about 150% or more of) those seen during a late treatment window (e.g. between T₄ₕᵣ and T₈ₕᵣ or T₄ₕᵣ and T₆ₕᵣ) after orally administering the reference Marinol capsule.

PK Profile 14: The PK profile of PK Profile 12 where the plasma levels are greater than (e.g. about 150% or more of) those seen during a mid-treatment window (e.g. between T₃ₕᵣ and T₅ₕᵣ) after orally administering the reference Marinol capsule.

PK Profile 15: The PK profile of PK Profile 12 where the plasma levels are substantially similar to (e.g. 50%-200%) of those seen during an early treatment window after orally administering the reference Marinol capsule.

PK Profile 16: The PK profile of PK Profile 12 where the plasma levels and/or an AUC during a mid treatment window (e.g. between T₃ₕᵣ and T₅ₕᵣ or at T₄ₕᵣ) are greater than about 75% of those seen during an early treatment window (e.g. between T₂ₕᵣ and T₄ₕᵣ or at T₃ₕ) after orally administering the reference Marinol capsule.

PK Profile 17: The PK profile of PK Profile 12 where the plasma levels and/or an AUC during a treatment window (e.g. between T₁ₕᵣ and Tₕᵣ) are substantially similar to (e.g. 50%-200% of) to those observed when two Marinol capsules containing about ½ the THC dose are orally administered in succession; e.g. one at T₀ and one at T₃ₕᵣ.

PK Profile 18: The PK profile of PK Profile 12 where the plasma levels and/or an AUC during a late treatment window (e.g. between T₄ₕᵣ and T₈ₕᵣ or T₄ₕᵣ and T₆ₕᵣ) are less than those observed when two Marinol capsules containing about ½ the THC dose are orally administered in succession; i.e. one at T₀ and one at T₃ₕᵣ).

PK Profile 19: The PK profile of PK Profile 12 where the plasma levels and/or an AUC during a late treatment window (e.g. between T₄ₕᵣ and T₈ₕᵣ or T₄ₕᵣ and T₆ₕᵣ) are substantially similar to [and optionally less than] (e.g. about 30%-100% of) those observed when two Marinol capsules containing about ½ the THC dose are orally administered in succession; i.e. one at T₀ and one at T₃ₕᵣ.

PK Profile 20: The PK profile of any one of PK Profile 1-19 where the plasma levels and/or an AUC during a late treatment window (e.g. between T₄ₕᵣ and Tₐₕᵣ or T₄ₕᵣ and T₆ₕ) are substantially similar to plasma levels and/or an AUC between T₈ₕᵣ and T₁₀ₕᵣ.

Medicaments

With the teachings provided herein, one skilled in the art can now provide a medicament that, when orally administered to a subject, produces a therapeutic response over a desired therapeutic window (e.g. extending over both an early treatment window and a late treatment window). In some embodiments, the formulations minimize the total amount of drug administered, thus significantly decreasing side effects and increasing the therapeutic efficiency.

The medicaments of the present invention comprise a cannabinoid partitioned between an immediate release compartment and a delayed release compartment. The portion of the cannabinoid in the immediate release compartment can be about any of the following per cents (%): 10-75, 10-50, 20-50, 25-75, 40-60, 35-75, or 40-80.

The compositions of the present invention comprise a cannabinoid and one or more excipients (e.g. release modifiers) in appropriate amounts to provide a desired PK profile (e.g. any of PK profiles 1-20). The component(s) of the compositions may be in any form, e.g. liquid, solid, and semi-solid components. The medicament can be formulated to provide any desired release properties, for example, immediate release, pulsatile release, extended release, delayed release, controlled-release, continuous release, prolonged release, timed release, and combinations thereof (e.g. immediate release+controlled-release, immediate release+delayed release, etc.).

The appropriate selection and amount of excipients is often influenced by the selection and amount (or fraction of the total dose) of cannabinoid(s) associated (e.g. compounded) with the excipients in a component (e.g. microparticle) of the dosage (and vice versa). With the teachings provided herein, one skilled in the art can now formulate medicaments which contain an excipient and release modifiers in an effective amount to provide both an immediate (or early) effect and an extended (or late) effect. For example, in one embodiment an immediate effect (or a substantial portion thereof) is provided while the sustained effect (or a substantial portion thereof) is provided by one or more sustained (e.g. extended or delayed) release modifiers. The skilled artisan will, of course, recognize that sustained effects (e.g. late plasma levels) may be influenced by an immediate release component in a dosage, for example, due to residual levels of the immediately released drug. However, with the teachings provided herein, one skilled in the art can adjust the release properties of one or more release components (e.g. by varying the amount of drug therein or the relative amounts or types of release modifiers therein).

The desired PK profiles can be achieved by the skilled artisan, in many cases, without performing clinical studies. For example, useful guidance is provided by the United States Food and Drug Administration (FDA) in the guidance document entitled “Extended Release Oral Medicaments: Development, Evaluation and Application of In Vitro/In Vivo Correlation”.

The medicaments of the present invention can be made with different polymorphic forms (e.g. salts, crystalline forms, hydrates, esters, and solvates), each with physicochemical properties affecting drug delivery (e.g. absorption). Selection of the release modifiers is done with consideration of the THC form. A number of such forms are well known in the art.

For example, in one embodiment of the invention, the cannabinoid form used in the formulation is a cannabinoid ester or salts thereof (e.g. a polar ester such as an ester of a terminal carboxylic acid). Esterified forms of THC are described, for example, in U.S. Pat. No. 4,933,368, U.S. Pat. No. 5,389,375 and U.S. Pat. No. 6,008,383. Other useful polar esters are the hemi-ester of malonic acid and the alaninate ester of alanine. It has been reported, e.g., in U.S. Pat. No. 5,508,051 and U.S. Pat. No. 5,389,375, that salts of the terminal carboxylic acid group of the ester, for example, the N-methyl glutamine salt as well as the sodium and potassium salts are also useful.

In another example, the cannabinoid form used in the formulation is in crystalline form. Optionally, the cannabinoid form is crystalline trans-(+/−)-THC. Examples of such crystalline forms are described, for example, in US 2007/0072939.

A medicament of the present invention can optionally provide a therapeutic window which extends over both an early treatment window and a late treatment window.

One embodiment of the present invention provides a medicament comprising a compartment comprising a cannabinoid and one or more excipients in an effective amount to provide an immediate therapeutic response (e.g. during an early-treatment window) and one or more release modifiers in an effective amount to provide an extended therapeutic window (e.g. providing a therapeutic response during a late-treatment window).

A medicament of the present invention comprises one or more dosage compartments.

A dosage compartment of the present invention can be a delayed release dosage compartment or an immediate release dosage compartment.

Optionally the delayed release dosage compartment comprises a liquid delayed release component (e.g. a delayed release aqueous or semi-aqueous component or syrup).

Optionally the delayed release dosage compartment comprises a solid delayed release component (e.g. an osmotic component, or a lyophilized or dried solid component selected from a lipid component, a glass sugar component, a guest-host component, or a co-precipitate component), or a semi-solid delayed release component (e.g. a semi-solid SEDDS component). Optionally, the solid delayed release dosage component or semi-solid delayed release dosage component is a component coated or complexed with a delayed release layer or compound, for example to provide a timed-release compartment.

Optionally, the delayed release dosage compartment comprises a solid component, for example, a lyophilized or dried, solid component selected from: a solid fat component, a glass sugar component, a guest-host component, and a co-precipitate component; wherein the solid component has been coated or otherwise compounded with a delayed (e.g. timed) release layer or delayed release modifier.

Optionally, a medicament comprises more than two or more compartments (e.g. 3, 4, 5, or 6 compartments), such as timed-release compartments having different times of cannabinoid release. Optionally, the medicament comprises an immediate release compartment and a plurality of delayed (e.g. timed) release compartments. Optionally, the plurality of delayed release compartments includes one or two or more delayed release compartments (e.g. 2, 3, or 4, with different release times) and/or one or more extended release compartments.

Liquid Dosage Compartments

In one embodiment, a dosage compartment of the present composition is a liquid or predominantly liquid (a liquid) dosage compartment. Any formulation useful for oily or lipophilic compounds may be used. For example, the component may be in the form of an aqueous or non-aqueous liquid, an oil or other lipophilic medium, an emulsion, a syrup, and the like. Optionally, a liquid compartment is encapsulated (e.g. a hard gel or soft gel). Optionally, the compartment is coated with a delayed (e.g. extended, sustained, or timed) release coating. The skilled artisan will readily appreciate that the liquid dosage compartments taught herein are amenable to delayed release or immediate release compartments, depending on, for example, the appropriate selection and amount of excipients.

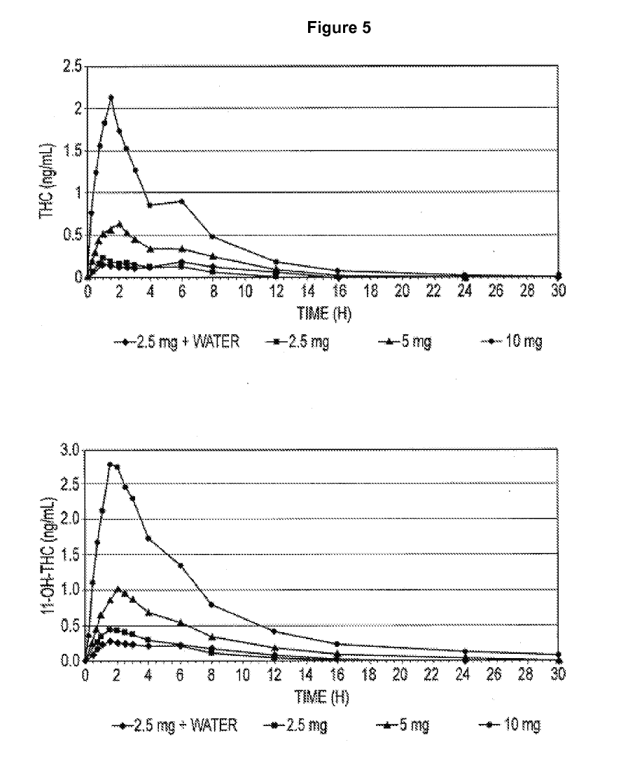

Optionally, the liquid dosage compartment of the present invention comprises an aqueous or semi-aqueous liquid component comprising a cannabinoid and organic co-solvents (e.g. ethanol, propylene glycol and polyethylene glycol). Optionally, the composition further comprises a buffer. An example of such a component is described in US 2009/0181080. For example, optionally a component contains from 15% to about 65% ethanol, from about 10% to about 60% buffered aqueous solution, from about 0.1 to about 25% propylene glycol and from about 1% to about 25% polyethylene glycol. Optionally, the component is buffered to a pH of about 6.5 to 7.5 (e.g. 7). Such an aqueous or semi-aqueous liquid component may be formulated for tailored (e.g. delayed) release. For example, FIG. 5 (reproduced from FIG. 6 of US 2009/0181080) shows the profile of several delayed release components that may be incorporated into a present dosage (e.g. by formulating with an immediate release component taught herein and/or varying the drug amount and/or selection or relative amounts of excipients in the component). In one embodiment, the liquid aqueous or semi-aqueous compartment is formulated as an immediate release compartment. In another embodiment, the liquid aqueous or semi-aqueous compartment is formulated as a delayed release compartment.

Optionally, the liquid dosage compartment of the present invention comprises a lipophilic medium (sometimes referred to simply as an ‘oil’) liquid component comprising a cannabinoid and a lipophilic medium, for example, an oil or oil-based carrier. Optionally, the oil comprises one or more triglycerides (e.g. a triglyceride component). Optionally, the oil comprises one or more phospholipids (e.g. a phospholipids component). Optionally, the oil comprises one or more triglycerides and one or more phospholipids. Optionally, the oil comprises a mixture of two or more of: phospholipids, glycolipids, triglycerides, sterols, small quantities of fatty acids, carbohydrates and sphingolipids, such as a complex mixture thereof (e.g. lecithin). Optionally, the oil comprises an antioxidant oil. Such oil-based carriers are readily available from commercial sources. Examples of suitable oils or oil-based carriers (e.g. triglyceride carriers) include, but are not limited to, Aceituno oil, Almond oil, Arachis oil, Babassu oil, Blackcurrant seed oil, Borage oil, Buffalo ground oil, Candlenut oil, Canola oil, Lipex 108 (Abitec), Castor oil, Chinese vegetable tallow oil, Cocoa butter, Coconut oil Pureco 76 (Abitec), Coffee seed oil, Corn oil, Cottonseed oil, Crambe oil, Cuphea species oil, Evening primrose oil, Grapeseed oil, Groundnut oil, Hemp seed oil, Illipe butter, Kapok seed oil, Linseed oil, Menhaden oil, Mowrah butter, Mustard seed oil, Oiticica oil, Olive oil, Palm oil, Palm kernel oil, Peanut oil, Poppy seed oil, Rapeseed oil, Rice bran oil, Safflower oil, Sal fat, Sesame oil, Shark liver oil, Shea nut oil, Soybean oil, Stillingia oil, Sunflower oil, Tall oil, Tea seed oil, Tobacco seed oil, Tung oil (China wood oil), Ucuhuba, Vernonia oil, Wheat germ oil, mixtures of any of the foregoing, and the like. Fractionated triglycerides, modified triglycerides, synthetic triglycerides, and mixtures of triglycerides are also within the scope of the invention. Other examples of oils, for example, triglycerides, include vegetable oils, fish oils, animal fats, hydrogenated vegetable oils, partially hydrogenated vegetable oils, medium and long-chain triglycerides, and structured triglycerides. Optionally, the vegetable oil is soybean oil, olive oil, cotton seed oil, peanut oil, sesame oil and castor oil, with sesame oil and castor oil optionally being preferred. Examples of useful phospholipids include: Phosal® 50 PG; Phosal® 53MCT; Phosal® 75SA, Phospholipon® 80; Phospholipon® 80H; Phospholipon® 85G; Phospholipon® 90G; Phospholipon® 90H; and Phospholipon® 90NG. Examplary phospholipids suitable for dermal medicaments include: Phosal® 50 PG; Phosal® 50SA; Phosal® 53MCT; Phosal® 75SA; Phospholipon® 80; Phospholipon® 80H; Phospholipon® 85G; Phospholipon® 90NG; Phospholipon® 90G; Phospholipon® 90H; and Phospholipon® 100H. Optionally, the oil component is a mixture of oils or complex mixture (as described above), for example, as with lecithin. Optionally, the lecithin comprises a mixture of phospholipids, for example, phosphatidylcholine (e.g. 13-18%); phosphatidylethonolamine (e.g. 10-15%); phosphatidyinositol (e.g. 10-15%); and phosphatidic acid (e.g. 5-12%). Vitamin E (tocopherol) can also be used in the oil component. Optionally, the oil-based carrier is sesame oil. Optionally, the oil component (e.g. sesame oil) contains an effective amount of an anti-oxidant selected from the group consisting of sesamin, sesamol, sesamolin, lecithin and any combination of the foregoing (either already present in the (unpurified) sesame oil or added to purified sesame oil. Examples of components described above are described in WO 2006/063109. In one embodiment, the liquid lipophilic compartment is formulated as an immediate release compartment. In another embodiment, the liquid lipophilic compartment is formulated as a delayed release compartment.

Optionally, the liquid dosage compartment of the present invention is a SEDDS dosage compartment comprising a cannabinoid, an oily medium, and at least one surfactant. An example of such a SEDDS dosage compartment is described in US 2007/0104741. The oily medium may comprise, for example, triglycerides and/or mixed glycerides and/or free fatty acids containing medium and/or long chain saturated, mono-unsaturated, and/or poly-unsaturated free fatty acids. The surfactant promotes self-emulsification, which, for example, promotes targeted chylomicron delivery and optimal availability to a mammalian intestinal lumen. The dosage compartment may, for example, include co-solvents, anti-oxidants, viscosity modifying agents, cytochrome P450 metabolic inhibitors, and P-GP efflux inhibitors, for tailored (e.g. delayed) release rates. Optionally, the SEDDS component is a Type I, II, or III SEDDS. Type I formulations contain an oily medium (e.g. triglycerides and/or mixed glycerides and/or medium/long chain saturated, mono-unsaturated, and poly-unsaturated free fatty acids); whereas the oily medium may also be polyfunctional with potential surfactant characteristics to promote self-emulsification. Type II contains an oily medium (e.g. triglycerides and/or mixed glycerides and/or medium/long chain saturated, mono-unsaturated, and poly-unsaturated free fatty acids) and at least one surfactant component to promote self-emulsification. Type III contains an oily medium (e.g. triglycerides and/or mixed glycerides and/or medium/long chain saturated, mono-unsaturated, and poly-unsaturated free fatty acids) and at least one surfactant component to promote self-emulsification, and at least one hydrophilic cosolvent. Optionally, the SEDDS compartment is coated with a release modifying coating (e.g. timed, extended, or delayed). In one embodiment, the liquid SEDDS compartment is formulated as an immediate release compartment. In another embodiment, the liquid SEDDS compartment is formulated as a delayed release compartment.

Optionally, the liquid dosage compartment is combined with another compartment in a multi-phase dosage form. For example, the liquid dosage compartment can be provided as an immediate release compartment and combined with a solid dosage compartment formulated for delayed release. Examples of useful multi-phase dosage forms are described in U.S. Pat. No. 7,670,612.

Semi-Solid Dosage Compartments

In one embodiment, a dosage compartment of the present composition is a semi-solid dosage compartment. Any formulation useful for oily or lipophilic compounds may be used. For example, the compartment may be in the form of self-emulsifying drug delivery system (SEDDS), or a lipophilic medium compartment. Optionally, the semi-solid compartment is encapsulated (e.g. a hard gel or soft gel). Optionally, the compartment is coated with a release modifying coating (e.g. timed release coating).

Optionally, the dosage compartment is a semi-solid SEDDS dosage compartment comprising a cannabinoid, an oily medium, at least one surfactant, and a semi-solid inducer (e.g. an amphiphilic/non-amphiphilic solute such as ascorbyl palmitate). Such a SEDDS component is described, for example, in US 2007/0104741. The oily medium may comprise, for example, triglycerides and/or mixed glycerides and/or free fatty acids containing medium and/or long chain saturated, mono-unsaturated, and/or poly-unsaturated free fatty acids. The surfactant promotes self-emulsification, which, for example, promotes targeted chylomicron delivery and optimal availability to a mammalian intestinal lumen. The amphiphilic/non-amphiphilic solute promotes prolonged dissolution profiles (e.g. for delayed release). The component may, for example, include co-solvents, anti-oxidants, viscosity modifying agents, cytochrome P450 metabolic inhibitors, P-GP efflux inhibitors, and finally amphiphilic/non-amphiphilic solutes to induce semi-solid formation for tailored (e.g. delayed) release rates. For example, an isotropic semi-solid or waxy solid phase may be prepared by dissolving a high concentration of ascorbyl palmitate (or other amphiphilic/non-amphiphilic solutes) in an oily liquid state. Upon administration as an isotropic semi-solid phase and upon initial dilution in the gastric region of a mammal, the contents immediately form a solid dispersion or coarse colloidal dispersion for protection against acid catalyzed degradation of cannabinoids. Optionally, the SEDDS component is a Type I, II, or III SEDDS. Type I formulations contain an oily medium (e.g. triglycerides and/or mixed glycerides and/or medium/long chain saturated, mono-unsaturated, and poly-unsaturated free fatty acids); whereas the oily medium may also be polyfunctional with potential surfactant characteristics to promote self-emulsification. Type II contains an oily medium (e.g. triglycerides and/or mixed glycerides and/or medium/long chain saturated, mono-unsaturated, and poly-unsaturated free fatty acids) and at least one surfactant component to promote self-emulsification. Type III contains an oily medium (e.g. triglycerides and/or mixed glycerides and/or medium/long chain saturated, mono-unsaturated, and poly-unsaturated free fatty acids) and at least one surfactant component to promote self-emulsification, and at least one hydrophilic cosolvent. Optionally, the SEDDS compartment is coated with a release modifying coating (e.g. delayed release coating or timed-release coating).

Optionally, the semi-solid dosage compartment of the present invention comprises a lipophilic (sometimes referred to simply as ‘oil’) semi-solid component comprising a cannabinoid and a lipophilic medium, for example, an oil or oil-based carrier. Optionally, the oil comprises one or more triglycerides (e.g. a triglyceride component). Optionally, the oil comprises one or more phospholipids (e.g. a phospholipids component). Optionally, the oil comprises one or more triglycerides and one or more phospholipids. Optionally, the oil comprises a mixture of two or more of: phospholipids, glycolipids, triglycerides, sterols, small quantities of fatty acids, carbohydrates and sphingolipids, such as a complex mixture thereof (e.g. Lecithin). Optionally, the oil comprises an antioxidant oil. Such oil-based carriers are readily available from commercial sources. Examples of suitable oils or oil-based carriers (e.g. triglyceride carriers) include, but are not limited to, Aceituno oil, Almond oil, Arachis oil, Babassu oil, Blackcurrant seed oil, Borage oil, Buffalo ground oil, Candlenut oil, Canola oil, Lipex 108 (Abitec), Castor oil, Chinese vegetable tallow oil, Cocoa butter, Coconut oil Pureco 76 (Abitec), Coffee seed oil, Corn oil, Cottonseed oil, Crambe oil, Cuphea species oil, Evening primrose oil, Grapeseed oil, Groundnut oil, Hemp seed oil, Illipe butter, Kapok seed oil, Linseed oil, Menhaden oil, Mowrah butter, Mustard seed oil, Oiticica oil, Olive oil, Palm oil, Palm kernel oil, Peanut oil, Poppy seed oil, Rapeseed oil, Rice bran oil, Safflower oil, Sal fat, Sesame oil, Shark liver oil, Shea nut oil, Soybean oil, Stillingia oil, Sunflower oil, Tall oil, Tea seed oil, Tobacco seed oil, Tung oil (China wood oil), Ucuhuba, Vernonia oil, Wheat germ oil, mixtures of any of the foregoing, and the like. Fractionated triglycerides, modified triglycerides, synthetic triglycerides, and mixtures of triglycerides are also within the scope of the invention. Other examples of oils, for example, triglycerides, include vegetable oils, fish oils, animal fats, hydrogenated vegetable oils, partially hydrogenated vegetable oils, medium and long-chain triglycerides, and structured triglycerides. Optionally, the vegetable oil is soybean oil, olive oil, cotton seed oil, peanut oil, sesame oil and castor oil, with sesame oil and castor oil optionally being preferred. Examples of useful phospholipids include: Phosal® 50 PG; Phosal® 53MCT; Phosal® 75SA, Phospholipon® 80; Phospholipon® 80H; Phospholipon® 85G; Phospholipon® 90G; Phospholipon® 90H; and Phospholipon® 90NG. Examplary phospholipids suitable for dermal medicaments include: Phosal® 50 PG; Phosal® 50SA; Phosal® 53MCT; Phosal® 75SA; Phospholipon® 80; Phospholipon® 80H; Phospholipon® 85G; Phospholipon® 90NG; Phospholipon® 90G; Phospholipon® 90H; and Phospholipon® 100H. Optionally, the oil component is a mixture of oils or complex mixture (as described above), for example, as with lecithin. Optionally, the lecithin comprises a mixture of phospholipids, for example, phosphatidylcholine (e.g. 13-18%); phosphatidylethonolamine (e.g. 10-15%); phosphatidyinositol (e.g. 10-15%); and phosphatidic acid (e.g. 5-12%). Vitamin E (tocopherol) can also be used in the oil component. Optionally, the oil-based carrier is sesame oil. Optionally, the oil component (e.g. sesame oil) contains an effective amount of an anti-oxidant selected from the group consisting of sesamin, sesamol, sesamolin, lecithin and any combination of the foregoing (either already present in the (unpurified) sesame oil or added to purified sesame oil. In one embodiment, the semi-solid oil compartment is formulated as an immediate release compartment. In another embodiment, the semi-solid oil compartment is formulated as a delayed release compartment. Examples of compartments described above are described in WO 2006/063109.

Solid Dosage Compartments

In one embodiment, a dosage compartment of the present invention is a solid dosage compartment. Any formulation useful for oily or lipophilic compounds may be used. For example, the dosage compartment may be a solid lipid dosage compartment, a solid dosage compartment produced from an aqueous mixture or emulsion, a solid dosage compartment produced by extrusion (e.g. hot melt extrusion), or a solid emulsion that is, for example, dried. Other solid dosage compartments include osmotic particles. Optionally, a solid dosage compartment (e.g. powdered, spray dried, or freeze dried forms) is formed into a tablet, pill, microsphere, and the like. Optionally, a solid dosage compartment is coated or otherwise compounded with pharmaceutically acceptable materials and/or excipients known in the art to provide a cannabinoid component affording delayed release (e.g. delayed release modifiers or timed-release coating).

Optionally, the solid dosage compartment is a solid lipid dosage compartment comprising a cannabinoid, a solid fat and one or more phospholipids, for example, as described in WO9736577. The dosage compartment may be prepared by dissolving the cannabinoid together with lipid components comprising at least one solid fat and at least one phospholipid in a suitable organic solvent; evaporating the solvent to dryness; hydrating the dry solid lipid mixture with an aqueous phase, with mechanical shaking, to obtain a lipid dispersion in water; homogenizing the resultant lipid dispersion, such as by high-pressure homogenization, to reduce the particle size to the submicron range; and drying the submicron dispersion. Alternatively, the dosage compartment may be prepared by directly drying the lipid mixture that is dissolved in the organic solvent. For example, the solid lipid mixture formulations can be spray dried or freeze-dried to obtain dry compositions suitable for the preparation of solid-dosage compartments, such as hard gelatin capsules or tablets. These solid dosage compartments may, for example, further comprise cryoprotectants, antioxidants, free flowing imparting agents, surface active materials and/or emulsifiers. In one embodiment, the solid lipid compartment is formulated as an immediate release compartment. In another embodiment, the solid lipid compartment is formulated as a delayed release compartment.

Optionally, the solid dosage compartment is a solubilizing compartment comprises a solubilizing agent (e.g. host such as a cyclodextrin) and the cannabinoid. An example of such a dosage compartment is described in WO9932107. The solid dosage compartment may be produced from an aqueous mixture or emulsion. The solubilizing action of, for example, cyclodextrins, is caused by the formation of so-called inclusion complexes or guest-host complexes, which may then be dried (e.g. freeze dried) to produce a powder material and optionally mixed with a microsphere (e.g. swelling starch microsphere). Other microspheres that may be used in the present compositions include those made from chitosan, polyvinylpyrrolidone, alginate, polycarbophil, pectin, hyaluronic acid (and esters thereof), agar agarose, dextran, albumin, ovalbumin, collagen, and casein. In one embodiment, the solid solubilizing compartment is formulated as an immediate release compartment. In another embodiment, the solid solubilizing compartment is formulated as a delayed release compartment.

Optionally, the solid dosage compartment is a co-precipitate compartment comprising a co-precipitate of a cannabinoid, a tocopherol polyethyleneglycol succinate (TPGS) or equivalent; and a dispersion adjuvant in an amount sufficient to assist in dispersing the lipophilic substance in the succinate. An example of such a dosage compartment is described for a cannabinoid in U.S. Pat. No. 5,891,469. The dosage compartment may be produced, for example, by co-melting TPGS and the cannabinoid at 40.degree.-60.degree. C.; adding a dispersion adjuvant to the melted mixture with agitation; adding a fumed silica to the mixture with agitation; and drying the resultant mixture at 100.degree. C. to yield a dry powder co-precipitate. In one embodiment, the solid co-precipitate compartment is formulated as an immediate release compartment. In another embodiment, the solid co-precipitate compartment is formulated as a delayed release compartment.

Optionally, the solid dosage compartment is a sugar glass compartment sugar comprising, a cannabinoid, and a glass of a sugar, a sugar alcohol, a mixture of sugars or a mixture of sugar alcohols. An example of such a dosage compartment is described in US 2003/0229027. Optionally, the cannabinoid is incorporated in the sugar glass as a monomolecular encapsulation without formation of a guest-host complex. The solid dosage compartment may, for example, be prepared by a) dissolving the cannabinoid compound in an organic solvent that is soluble in water and dissolving the sugar, sugar alcohol, mixture of sugars or mixture of sugar alcohols in water; b) mixing the dissolved cannabonoid compound and the dissolved sugar, sugar alcohol, mixture of sugars or mixture of sugar alcohols to obtain a sufficiently stable mixture; and c) drying the mixture by freeze drying, spray drying, vacuum drying, or super critical drying. The dissolution rate of the cannabinoid may be tailored by manipulating the dissolution rate of the sugar glass, for example, to produce delayed release. In one embodiment, the solid sugar glass compartment is formulated as an immediate release compartment. In another embodiment, the solid sugar glass compartment is formulated as a delayed release compartment.

Optionally, the solid dosage compartment is a hot melt extrudate. An examplary method of making a solid hot melt extrudate comprises mixing a cannabinoid and a carrier by a rotating screw through the heated barrel of an extruder and pressing the melt through a die into a product of uniform shape. Useful carriers include polymers known in the art as extrudate carriers. For example, the carrier can be a hydrophobic and/or hydrophilic fusible carrier or diluent (e.g. with a melting point from 35° C. 150° C.). As another example, the carrier can be a high molecular weight poly(ethylene oxide). As another example, the carrier can comprise a polymer matrix comprising a thermoplastic polymer or lipophilic carrier. Optionally, the carrier further comprises a plasticizer. Optionally, the carrier further comprises a delayed release modifier. Optionally, the polymers used in the extrusion process function as thermal binders, drug stabilizers, drug solubilizers and/or drug release controlling excipients with no compressibility requirements. Examples of useful polymer carriers include vinyl polymers (polyvinylpyrrolidone [PVP]), PVP-vinyl acetate [PVP-VA]), poly(ethylene oxide) (PEO), Eudragit (acrylates), PE glycol (PEG) and cellulose derivatives (hydroxypropylcellulose [HPC], hydroxypropyl methylcellulose [HPMC], HPMC acetate succinate [HPMCAS], cellulose acetate [CA] and CA-phthalate [CAP]). Other examples of useful hot melt extrusion carriers or methods are described in U.S. Pat. No. 6,488,963 B1, US 20100047340, US 20080274194, U.S. Pat. No. 6,375,963, U.S. Pat. No. 7,771,632, U.S. Pat. No. 6,743,442, and U.S. Pat. No. 5,849,240. In one embodiment, the hot melt extrudate compartment is formulated as an immediate release compartment. In another embodiment, the hot melt extrudate compartment is formulated as a delayed release compartment (e.g. sustained release or timed release).

Optionally, the solid dosage compartment is an osmotic compartment comprising an osmotic dosage compartment comprising a layered core (e.g. single layer or bilayer core) comprising a cannabinoid and an expandable polymer; a semipermeable membrane surrounding the core; and a passageway disposed in the semipermeable membrane for tailored (e.g. delayed) release of the cannabinoid. Optionally, the core is a multilayer (e.g. bilayer) comprising first layer comprising the cannabinoid and excipient (e.g. low molecular weight polymers) and a second layer comprising an osmopolymer. An example of such a dosage compartment is described in WO 2008/024490. Optionally, the second layer further comprises an osmoagent. Optionally, the one or more outer layers also comprise a cannabinoid to provide a more immediate release in addition to the tailored release. Useful osmopolymers include high molecular weight polyoxyethylene oxides or the derivatives thereof. Other useful osmopolymers include hydrogels such as Carbopol® acidic carboxy polymers, a polymer of acrylic acid crosslinked with a polyallyl sucrose, also known as carboxypolymethylene and carboxyvinyl polymer having a molecular weight of 250,000 to 4,000,000; Cyanamer® polyacrylamides; cross-linked water swellable indene-maleic anhydride hydrogel polymers; Good-rite® polyacrylic acid having a molecular weight of 80,000 to 200,000; Polyox® polyethylene oxide polymers having a molecular weight of 100,000 to 5,000,000 and higher; starch graft copolymers; Aqua-Keeps® acrylate polymer polysaccharides composed of condensed glucose units such as diester cross-linked polyglucan; and the like. Useful osmoagents include soluble salts of inorganic acids, such as magnesium chloride or sulphate, lithium, sodium or potassium chloride; soluble salts of organic acids, such as sodium or potassium acetate, magnesium succinate, sodium benzoate, sodium citrate, sodium ascorbate; carbohydrates, such as arabinose, ribose, xylose, glucose, fructose, galactose, mannose, sucrose, maltose, lactose, raffinose; hydrosoluble aminoacids, such as glycine, leukine, alanine, methionine; organic polymeric osmoagents, such as sodium carboxymethylcellulose, hydroxypropylmethylcellulose, hydroxyethylmethylcellulose, polyvinylpirrolidone, polyoxyethylene oxide, carbomers and polyacrylamides. Examples of useful osmotic dosage layers and components are described in U.S. Pat. No. 6,919,373, U.S. Pat. No. 6,930,129, U.S. Pat. No. 5,264,446, U.S. Pat. No. 5,674,895, U.S. Pat. No. 5,840,754, US 2010/0143472, U.S. Pat. No. 4,327,725, U.S. Pat. No. 4,111,202, and WO 2008/024490.

Optionally, the solid dosage compartment comprises a crystalline cannabinoid form. Optionally, the crystalline form is a mixture of cannabinoid enantiomers. Optionally, the crystalline form is a mixture of trans-(−)-THC and trans-(+)-THC (e.g. dronabinol and its (+) enantiomer). Optionally, the mixture is a racemic mixture. Examples of such crystalline forms are described in US 2007/0072939. It is interesting to note that US 2007/0072939 describes the trans-(−) entantiomer (dronabinol) as the more potent of the two, and that the activity of the trans-(+) enantiomer is only about 1% compared to the trans-(−)-enantiomer. This, however, has not been shown for sleep related disorders taught herein (e.g. apnea) or the activity associated with treatment. When formulating a medicament comprising a mixture of trans-(−)-THC and trans-(+)-THC, one embodiment provides that only trans-(−)-THC (e.g. dronabinol) is considered for PK profile evaluation and drug dose or dosage amount. In alternative embodiment, both the trans-(−)-THC and trans-(+)-THC are considered, but values for the trans-(+)-THC enantiomer are multiplied by a coefficient of about 1% to about 10% (e.g. multiplied by coefficient of about 1% to correct for its hypothetically reduced activity). In one embodiment, the crystalline compartment is formulated as an immediate release compartment. In another embodiment, the crystalline compartment is formulated as a delayed release compartment (e.g. sustained release or timed release).

Optionally, the solid dosage compartment is a delayed release compartment comprising a gastroretentive compartment. Gastroretentive compartments can comprise, for example, a water-swellable polymer that swells upon imbibition of water to a size that is large enough to promote retention of the dosage form in the stomach, and provides controlled delivery of the cannabinoid (e.g. by diffusion). Examples of useful gastroretentive compartments are described in WO 03/035029 A1, U.S. Pat. No. 6,635,280 B2, U.S. Pat. No. 6,340,475 B2, U.S. Pat. No. 6,488,962 B1, and PCT WO 2007/052125 A2.

Optionally, the solid dosage compartment is combined with another compartment in a multi-phase dosage form. For example, the solid dosage compartment can be provided as a delayed release compartment and combined with a liquid dosage compartment formulated for immediate release. Examples of useful multi-phase dosage forms are described in U.S. Pat. No. 7,670,612.

Delayed Release Coatings

As described herein, medicaments of the present invention may include a delayed release compartment. While any delayed release compartment is useful in the present invention, one embodiment provides a dosage compartment, wherein the compartment comprises a delayed release coating (e.g. sustained, extended or pulsatile release coating). For example, one or more pulsatile release coatings can be provided to deliver a burst of cannabinoid release at one or more predetermined time intervals (e.g. a burst at a time between about 1 and 5 hrs, 2 and 4 hrs, or 2.5-3.5 hrs, such as a burst at 2 hrs, 2.5 hrs, 3 hrs, 3.5 hrs, or 4 hrs).

Any dosage compartment taught herein may comprise a delayed release coating in order to provide a delayed release compartment. For example, an otherwise immediate release compartment (e.g. an immediate release liquid soft gel or hard gel) may be converted into a delayed release compartment by coating the compartment with a delayed release coating (i.e. a delayed release modifier coating). Additionally or alternatively, a delayed release compartment of the invention (e.g. a delayed release liquid soft gel or hard gel) may be coated in order to further modify its release properties. The coated compartment may be a solid compartment, liquid compartment, or semi-solid compartment.

By way of example, the delayed release coating may be a continuous coat which encapsulates the compartment as a whole (e.g. surrounding an entire tablet compartment, hard gel compartment or microparticles compartment as a whole). As an alternative example, the delayed release coating may be a discontinuous coating, and coat the compartment by individually encapsulating components of the compartment (e.g. by surrounding individual microparticles, hard gels, or tablets which make up the compartment). For example, a discontinuous delayed release coating is optionally useful when one compartment (e.g. solid microparticles) is mixed with another compartment (e.g. an immediate release liquid compartment) because it further segregates the compartments (in the medicament and/or once ingested).

Any release modifying coating (e.g. extended, pulsatire, or delayed) may be used to encapsulate a compartment taught herein. Examples of such are well known in the art. As an illustrative embodiment, the coating may comprise a hydrophobic release modifier, for example, an alkylcellulose (e.g. ethyl cellulose), an acrylic polymer, mixture thereof, and the like. Optionally, the delayed release coating is applied in the form of an organic or aqueous solution or dispersion. Optionally, the delayed release coating comprises a plasticizer, or other excipient, such as optional excipients taught herein. Optionally, the delayed release (e.g. pulsatile release) coating is pH dependent. Optionally, the delayed release coating is a water swellable sustained release coating. Optionally, the delayed release coating is low permeability coating or a high permeability coating, such as those described herein. Optionally, the delayed release coating is a low melting point hydrophobic material (e.g. wax).

A non-limiting example of a useful alkylcellulosic polymer is ethylcellulose, although the artisan will appreciate that other cellulose and/or alkylcellulose polymers may be readily employed, singly or in any combination, as all or part of a delayed release coating according to the invention. One commercially-available aqueous dispersion of ethylcellulose is Aquacoat® (FMC Corp., Philadelphia, Pa., U.S.A.). Aquacoat® is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. Prior to using the same as a coating, one may intimately mix the Aquacoat® with a suitable plasticizer. Another aqueous dispersion of ethylcellulose is commercially available as Surelease® (Colorcon, Inc., West Point, Pa., U.S.A.). This product is prepared by incorporating plasticizer into the dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion which can be applied directly onto substrates.

Examples of useful acrylic polymers include acrylic acid and methacrylic acid copolymers, methyl methacrylate copolymers, ethoxyethyl methacrylates, cynaoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

Optionally, a delayed release acrylic polymer is comprised of one or more ammonio methacrylate copolymers. Ammonio methacrylate copolymers are well known in the art, and are described, for example, as fully polymerized copolymers of acrylic and methacrylic acid esters with a low content of quaternary ammonium groups. In order to tailor a dissolution profile, one may, for example, incorporate two or more ammonio methacrylate copolymers having differing physical properties, such as different molar ratios of the quaternary ammonium groups to the neutral (meth)acrylic esters.

In one embodiment, the delayed release coating is pH dependent. Optionally, the pH dependent delayed release coating is a pulsatile release coating to deliver a burst of cannabinoid release at a predetermined time (e.g. upon entering the gut). Optionally, the pH dependent delayed release coating is a water-swellable coating. Optionally, the amount of water absorbed by the coating is pH-dependent. Optionally, the delayed release coating does not swell at about pH<5.7 and is soluble at about pH>6. Optionally, the delayed release coating does not swell at about pH<6.5 and is soluble at about pH>7. Methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit® from Evonik. There are several different types of Eudragit®. For example, Eudragit E is an example of a methacrylic acid copolymer which swells and dissolves in acidic media. Eudragit L is a methacrylic acid copolymer which does not swell at about pH<5.7 and is soluble at about pH>6. Eudragit S does not swell at about pH<6.5 and is soluble at about pH>7. Eudragit RL and Eudragit RS are water swellable, and the amount of water absorbed by these polymers is pH-dependent, however, medicaments coated with Eudragit RL and RS may be pH-independent.

Optionally, a useful acrylic delayed release coating comprises a copolymer of acrylic and methacrylic esters with a low content of quaternary ammonium groups, for example, having a molar ratio of ammonium groups to the remaining neutral (meth)acrylic esters of 1:20 to 1:40, such as 1:20 or 1:40. Optionally, a useful acrylic delayed release coating comprises a mixture of two acrylic resin lacquers, e.g. those commercially available from Evonik under the Tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. The code designations RL (high permeability) and RS (low permeability) refer to the permeability properties of these agents. Eudragit® RL/RS mixtures are insoluble in water and in digestive fluids. However, coatings formed from the same are swellable and permeable in aqueous solutions and digestive fluids. The Eudragit® RL/RS dispersions of the present invention may be mixed together in any desired ratio in order to ultimately obtain a delayed-release formulation having a desirable profile. Optional delayed-release formulations may be obtained, for instance, from a retardant coating derived from 100% Eudragit® RL, 50% Eudragit® RL:50% Eudragit® RS, and 10% Eudragit® RL:Eudragit® 90% RS. Of course, one skilled in the art will recognize that other acrylic polymers may also be used, such as, for example, Eudragit®L.

In one embodiment, a delayed release coating comprises a plasticizer. For example, where the coating is produced with an aqueous dispersion of a hydrophobic delayed release material, an effective amount of a plasticizer is optionally provided to further improve the physical properties of the release modifying coating. For example, because ethylcellulose has a relatively high glass transition temperature and does not form flexible films under normal coating conditions, one may incorporate a plasticizer into an ethylcellulose release coating (or other alkylcellulose or other delayed release coating) before using the same as a coating material. Generally, the amount of plasticizer included in a coating solution is based on the concentration of a film-former, e.g., most often from about 1 to about 50 percent by weight of the film-former. Concentration of the plasticizer, may be determined based on routine experimentation with the particular coating solution and method of application. Examples of suitable plasticizers for ethylcellulose and the like include dibutyl sebacate, diethyl phthalate, triethyl citrate, tibutyl citrate, and triacetin, although it is possible that other plasticizers (such as acetylated monoglycerides, phthalate esters, castor oil, etc.) may be used. Examples of suitable plasticizers for the acrylic polymers of the present invention include, but are not limited to citric acid esters such as triethyl citrate, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Optionally, triethyl citrate is used as a plasticizer for the aqueous dispersions of ethyl cellulose of the present invention.

In one embodiment, a delayed release coating comprises a small amount of talc. This may, for example, reduce the tendency of the aqueous dispersion of a hydrophobic delayed release coating to stick during processing, and may additionally act as a polishing agent.

Other examples of useful delayed release coatings and useful dosage forms which comprise delayed release coatings are described in U.S. Pat. No. 7,670,612.

Optional Excipients

Component and/or composition properties, for example, bulk stability, dissolution and other release properties of composition components (e.g. solid, liquid, or semi-solid dosage compartments) may be manipulated by choosing an appropriate excipient, amount thereof, or formulation method using such. Non-limiting examples of pharmaceutical excipients include:

(a) Binders such as acacia, alginic acid and salts thereof, cellulose derivatives, methylcellulose, hydroxyethyl cellulose, hydroxypropyl cellulose, magnesium aluminum silicate, polyethylene glycol, gums, polysaccharide acids, bentonites, hydroxypropyl methylcellulose, gelatin, polyvinylpyrrolidone, polyvinylpyrrolidone/viny-I acetate copolymer, crospovidone, povidone, polymethacrylates, hydroxypropylmethylcellulose, hydroxypropylcellulose, starch, pregelatinized starch, ethylcellulose, tragacanth, dextrin, microcrystalline cellulose, sucrose, or glucose, and the like.

(b) Disintegration agents such as starches, pregelatinized corn starch, pregelatinized starch, celluloses, cross-linked carboxymethylcellulose, crospovidone, cross-linked polyvinylpyrrolidone, a calcium, a sodium alginate complex, clays, alginates, gums, or sodium starch glycolate, and any disintegration agents used in tablet preparations.

(c) Filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

(d) Surfactants such as sodium lauryl sulfate, sorbitan monooleate, polyoxyethylene sorbitan monooleate, polysorbates, poloxamers, bile salts, glyceryl monostearate, Pluronic™ line (BASF), and the like.

(e) pH correcting agents (buffers) such as citric acid, succinic acid, fumaric acid, malic acid, tartaric acid, maleic acid, glutaric acid, their alkaline salts, sodium bicarbonate and sodium carbonate and the like.

(f) Stabilizers such as any antioxidation agents, buffers, or acids, and the like, can also be utilized.

(g) Lubricants such as magnesium stearate, calcium hydroxide, talc, sodium stearyl fumarate, hydrogenated vegetable oil, stearic acid, glyceryl behenate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like.

(h) Wetting agents such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like.

(i) Diluents such lactose, starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, or bentonite, and the like.

(j) Anti-adherents or glidants such as talc, corn starch, DL-leucine, sodium lauryl sulfate, and magnesium, calcium, or sodium stearates, and the like.

(k) Pharmaceutically compatible carriers comprising acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, or pregelatinized starch, and the like.

(l) delayed release modifiers (e.g. selected from optional excipients) such as delayed release coatings, extended release coatings, and extended release components which are complexed with a cannabinoid component.

Combinations

The present medicaments can optionally be combined with one or more additional therapeutic agents. Optionally, the one or more additional therapeutic agents can be provided as immediate release agents, delayed release agents, or a combination thereof.

The present medicaments can optionally be combined with therapeutic agents useful in the treatment of a cannabinoid-sensitive disorder. For example, the one or more additional therapeutic agents are optionally therapeutic agents useful in the treatment of cannabinoid-sensitive disorder selected from: apnea, seizures, a neurological disorder, a pain disorder, an appetite or wasting disorder, nausea, vomiting, a sleep disorder, a breathing disorder, or a sleep-related breathing disorder.

Optionally, the present medicaments are combined with one or more anti-apnea therapeutic agents, for example, any of: serotonin reuptake inhibitors, serotonin receptor antagonists, serotonin receptor (e.g. subtype 1) agonists, serotonin agonists, noradrenalin reuptake inhibitors, combined serotonin/noradrenalin reuptake inhibitors, glutamate receptor antagonists, glutamate antagonists, inhibitors of glutamate release, glycine antagonists, GABA receptor agonists, calcitonin gene-related peptide (CGRP) receptor antagonists or release inhibitors, adenosine, adenosine analogs and nucleoside (e.g. adenosine) uptake blockers or reuptake inhibitors, opioid antagonists, vanilloid receptor ligands, pilocarpine compounds, sodium proton pump inhibitors, ubidecarenones, antihistimines, prostaglandins, prostanoid receptor antagonists, inhibitors of prostanoid synthesis, modulators of CRTH2, COX-2 and/or FAAH, antitussive agents, compounds that stimulate the central nervous system, agents that prolong the action of endocannabimimetics, inhibitors of endocannabinoid membrane transport, inhibitors of cannabinoid metabolism, and cannabinoid degradative enzyme antagonists.

Optionally, the present medicaments are combined with one or more anti-apnea therapeutic agents or combinations of apnea therapeutic agents disclosed in any of: U.S. Pat. No. 6,331,536, U.S. Pat. No. 6,555,564, U.S. Pat. No. 6,727,242, U.S. Pat. No. 7,160,898, U.S. Pat. No. 6,974,814, U.S. Pat. No. 7,705,039, US20060241164, US20070123517, US20080200367, US20080261922, US20090005357, US20090221658, US20100137251, EP1066036, EP1372638, EP1572202, EP1868602, JP2010059195, WO/1999/043319, WO/2000/051590, WO/2002/080903, WO/2004/041272, WO/2006/113448, WO/2006/133197, WO/2007/047372, WO/2007/047575, WO/2007/047576, WO/2007/047577, which disclose useful examples of the anti-apnea therapeutic agents and/or other anti-apnea therapeutic agents.

Optionally, the present medicaments are combined with one or more anti-convulsants, for example, anti-convulsants of any of the following types: aldehydes, aromatic allylic alcohols, barbiturates, benzodiazepines, bromides, carbamates, carboxamides, fatty acids, fructose derivatives, gaba analogs, hydantoins, oxazolidinediones, propionates, pyrimidinediones, pyrrolidines, succinimides, sulfonamides, triazines, ureas, and valproylamides (amide derivatives of valproate).

Optionally, the present medicaments are combined with one or more analgesics, for example, any of: NSAIDs (e.g. ibuprofen), paracetamol, COX-1 inhibitors, COX-2 inhibitors, COX-3 inhibitors, opioids (e.g. hydrocodone or oxycodone), morphinomimetics, flupirtine, tricyclic antidepressants (e.g. amitriptyline), tetracyclic antidepressants, anticonvulsants (e.g. carbamazepine, gabapentin, or pregabalin), anticholinergics, antispasmodics, K+ channel openers, NMDA receptor antagonists (e.g, dextromethorphan, ketamine, and amantadine), steroids, anti-inflammatories, non-narcotic analgesic (e.g. tramadol), NK1 receptor antagonists (e.g., ezlopitant and SR-14033, SSR-241585), CCK receptor antagonists (e.g., loxiglumide), NK3 receptor antagonists (e.g., talnetant, osanetant SR-142801, SSR-241585), norepinephrine-serotonin reuptake inhibitors (NSRI; e.g., milnacipran), vanilloid receptor agonists and antagonists, cannabinoid receptor agonists (e.g., arvanil), sialorphin, inhibitors of neprilysin, CCK receptor agonists (e.g., caerulein), SSRIs (e.g. fluoxetine, paroxetine, or sertraline), serotonin receptor agonists, serotonin receptor antagonists, triptans (e.g. sumatriptan), GABA analogs (e.g. GABApentin or pre-gabalin), muscle relaxants, alpha-adrenergic, PDEV inhibitors, PDEVII inhibitors, and glycine antagonists.

Optionally, the present medicaments are combined with one or more anxiolytic or anti-anxiety therapeutic agents, for example, any of: benzodiazepines, buspirone, tricyclic antidepressants, SSRIs, monoamine oxidase inhibitors, antipsychotic agents, antihistamines (e.g. Atarax or Vistaril), barbiturates (e.g. phenobarbital), and beta-blockers (e.g. propranolol), and propanediols (e.g. meprobamate).

Optionally, the present medicaments are combined with one or more anti-wasting therapeutic agents or appetite stimulants, for example, any of: tricyclic antidepressants, tetracyclic antidepressants, cyproheptadine, buclizine, megestrol, ginger, EPA (fish oil), erthythropoietin, thalidomide, ghrelin, interferon, melatonin, non-steroidal anti-inflammatories, nandrolone, antidepressants, atypical antipsychotics such as olanzapine, dexamethasone, prednisolone and methylprednisolone.

Optionally, the present medicaments are combined with one or more anti-glaucoma therapeutic agents, e.g., fish oil and omega 3 fatty acids, bilberries, vitamin E, cannabinoids, carnitine, coenzyme Q10, curcurmin, Salvia miltiorrhiza, dark chocolate, erythropoietin, folic acid, Ginkgo biloba, Ginseng, L-glutathione, grape seed extract, green tea, magnesium, melatonin, methylcobalamin, N-acetyl-L cysteine, pycnogenols, resveratrol, quercetin and salt, magnesium, ginkgo, salt and fludrocortisone.

Optionally, the present medicaments are combined with one or more anti-emetics, e.g., serotonin receptor antagonist, dopamine antagonist, NK1 receptor antagonist, antihistamine, benzodiazepine, anticholinergic, or steroid such as dexamethasone.

Optionally, the present medicaments are combined with one or more additional therapeutic agents selected from: antihistamines, antimicrobial agents, fungistatic agents, germicidal agents, hormones, antipyretic agents, antidiabetic agents, bronchodilators, antidiarrheal agents, antiarrhythmic agents, coronary dilation agents, glycosides, spasmolytics, antihypertensive agents, antidepressants, antianxiety agents, antipsychotic agents, other psychotherapeutic agents, steroids, corticosteroids, analgesics, cold medications, vitamins, sedatives, hypnotics, contraceptives, nonsteroidal anti-inflammatory drugs, blood glucose lowering agents, cholesterol lowering agents, anticonvulsant agents, other antiepileptic agents, immunomodulators, anticholinergics, sympatholytics, sympathomimetics, vasodilatory agents, anticoagulants, antiarrhythmics, prostaglandins having various pharmacologic activities, diuretics, sleep aids, antihistaminic agents, antineoplastic agents, oncolytic agents, antiandrogens, antimalarial agents, and antileprosy agents.

The present medicaments can optionally be combined with one or more SSRIs, e.g., fluoxetine, paroxetine, fluvoxamine, sertraline, citalopram, norfluoxetine, r(−) fluoxetine, s(+) fluoxetine, demethylsertraline, demethylcitalopram, venlafaxine, milnacipran, sibutramine, nefazodone, R-hydroxynefazodone, (−)venlafaxine, and (+) venlafaxine.

The present medicaments can optionally be combined with one or more serotonin receptor antagonists, e.g., the free base form or a quaternized form of zatosetron, tropisetron, dolasetron, hydrodolasetron, mescaline, oxetorone, homochlorcyclizine, perlapine, ondansetron (GR38032F), ketanserin, loxapine, olanzapine, chlorpromazine, haloperidol, r (+) ondansetron, cisapride, norcisapride, (+) cisapride, (−) cisapride, (+) norcisapride, (−) norcisapride, desmethylolanzapine, 2-hydroxymethylolanzapine, 1-(2-fluorophenyl)-3-(4-hydroxyaminoethyl)-prop-2-en-1-one-0-(2-dimethylaminoethyl)-oxime, risperidone, cyproheptadine, clozapine, methysergide, granisetron, mianserin, ritanserin, cinanserin, LY-53,857, metergoline, LY-278,584, methiothepin, p-NPPL, NAN-190, piperazine, SB-206553, SDZ-205,557,3-tropanyl-indole-3-carboxylate, 3-tropanyl-indole-3-carboxylate methiodide, and other serotonin receptor antagonists and their quaternized forms or one of its pharmaceutically acceptable salts.

The present medicaments can optionally be combined with one or more serotonin receptor agonists, e.g., 8-OH-DPAT, sumatriptan, L694247 (2-[5-[3-(4-methylsulphonylamino)benzyl-1,2,4-oxadiazol-5-yl]-1H-indol-3yl]ethanamine), buspirone, aInitidan, zalospirone, ipsapirone, gepirone, zolmitriptan, risatriptan, 311C90, α-Me-5-HT, BW723C86 (1-[5(2-thienylmethoxy)-1H-3-indolyl[propan-2-amine hydrochloride), and MCPP (m-chlorophenylpiperazine).

The present medicaments can optionally be combined with one or more α 2 adrenergic receptor antagonists, e.g., phenoxybenzamine, phentolamine, tolazoline, terazosine, doxazosin, trimazosin, yohimbine, indoramin, ARC239, and prazosin.

The present medicaments can optionally be combined with one or more noradrenalin reuptake inhibitors, e.g. desipramine, nortriptyline, reboxetine, nisoxetine, atomoxetine, or LYI 39603 (tomoxetine).

The present medicaments can optionally be combined with one or more antitussive agents, e.g., RSD931, FK888, CP99994, SR48968, codeine, SR142801, SB235375, nociceptin/orphanin FQ, 15-HPETE, NADA, anandamide, lidocaine, benzonatate, mexiletine, NS1619, furosemide, zafirlukast, HOE140, dihydrocodone, oxycodone, BW443C noscapine, dextromethorphan, SKF10047, baclofen, diphenhydramine, caramiphen, glaucine, cilomilast, RO-64-6198, NKP608, levchromkalim, BRL55834, icatibant, suplatast tosilate, epinastine, levodropropizine, and other antitussive agents {Trends in Pharm Sci 25: 569-576, 2004; Br J Pharmacol 117:853-858, 1996).

The present medicaments can optionally be combined with one or more CCK receptor antagonists, e.g. CCK A receptor antagonist, a CCK B receptor antagonists, or an antagonist exhibits activity against both CCK A and CCK B receptors. Examplary antagonists which exhibit activity toward both CCK A and CCK B receptors include benzotript and proglumide. Examplary CCK A receptor antagonists include L-364,718 (devazepide); loxiglumide; dexloxiglumide; lorglumide; L-lorglumide; D-lorglumide; PD-140,548; TP-680; T-0632; A-67396; A-70276; A-71134 and SR 27897. Examplary CCK B receptor antagonists include CR2945; YMO22; itriglumide; L-740,093; L-365,260; L-156,586; LY-262691; ureidoacetamides (e.g., RP 69758, RP 72540, RP 73870); tetronothiodin; peptide analogs (CI-1015 and CI-988); YF476; A-63387 and GV150013X. Other examplary CCK receptor antagonists include, but are not limited to, A-64718; A-65186; spiroglumide; CR-2345; CR-2767; CR2622; tarazepide; L-365,260; L-708,474; L-368,730; L-369,466; L-736,380; FK-480; FR175985; FR193108; FR196979; FR202893, FR208418; FR208419; CP212,454; CP310,713; GV191869X; GV199114X; RPR1011367; S-0509; DA-3934; D51-9927; LY-202769; CCK-8; CCK-4; CAM1189; PD-135,666; CAM1481; PD-140,547; PD-140,723; PD-149,164; JB93182; AG-041 R; SR-27,897 (linitript); KSG-504; and 2-NAP.

The present medicaments can optionally be combined with one or more NSAIDs, e.g., aspirin, choline and magnesium salicylates, choline salicylate, celecoxib, diclofenac potassium, diclofenac sodium, diclofenac sodium with misoprostol, diflunisal, etodolac, fenoprofen calcium, flurbiprofen, ibuprofen, indomethacin, ketoprofen, magnesium salicylate, meclofenamate sodium, mefenamic acid, meloxicam, nabumetone, naproxen, naproxen sodium, oxaprozin, piroxicam, rofecoxib, salsalate, sodium salicylate, sulindac, tolmetin sodium, or valdecoxib.

The present medicaments can optionally be combined with one or more prostanoid receptor antagonists, e.g., AH-6809, ONO-8711 and ONO-8713, L-161,982 (4), AH-23848 (4), ONO-AE3-208 (4), SC19220, ONO-8711, SC51089, L-798,106, prostanoid receptor antisense oligonucleotides, ONO-8713, ONO-AE829, ONO-AE2-227, SC51322, ZD-6416, ONO-3144, ONO-3708, ONO-NT-12, SC-236, SC-299, SC-51234A, SKF-104493, SKF-105561, SKF-105809, SKF-106978, SKF-86002, SQ-28852, CGP-47969A, CGS-11776, FR-122047, L-640035, L-651392, L-651896, L-655240, L-657925, L-657926, L-745296, L-745337, L-746483, L-761066, L-768277, PD-138387, WY-48090, S-5751, BWA868C, ramatroban, Ifetroban, SQ-29548, ICI-192605, S-1452, FCE-27262, ONO-8809, ONO-NT-126, CBS-1108, L-636499, diphloretin phosphate, polyphloretin phosphate, BM-613, BM-500, BM-519, BM-567, BM-573, ONO-3708, seratrodast, GW627368 and taprostene, YM-158, YM-26734, YM-57158, Z-335, ZD-9583, 6-[N-(2-benzyloxy-5-bromobenzyl)-N-ethylamino]pyridazine-3-carboxylic acid, 6-[N-(5-bromo-2-(2-methylprop-2-en-1-yloxy) benzyl)-N-ethylamino]pyridazine-3-carboxylic acid (disclosed in Example 15 of International Patent Publication No. WO 97/00864), N-propanesulphonyl-6-[N-(5-bromo-2-(cyclopropylmethoxy)benzyl)-N-ethylamino]pyridazine-3-carboxamide (disclosed in Example 14 of International Patent Publication No. WO 97/00863), N-(3,5-dimethylisoxazol-4-ylsulphonyl)-6-[N-(5-chloro-2-(2-methylpropoxy)benzyl-N-ethylamino]pyridazine-3-carboxamide (disclosed as compound number 1 in Example 8 of International Patent Publication No. WO 97/00863), 6-[N-(5-bromo-2-(cyclopropylmethoxy) benzyl)-N-ethylamino]pyridazine-3-carboxylic acid (disclosed in Example 3 of International Patent Publication No. 97/00863), and the like.

The present medicaments can optionally be combined with one or more CGRP receptor antagonists, e.g., BIBN4096BS, SB-(+)-273779, CGRP₈₋₃₇, Compound 1 (4-(2-oxo-2,3-dihydro-benzoimidazol-1-ŷ-piperidine-1-carboxylic acid [1-(3,5-dibromo-4-hydroxy-benzyl)-2-oxo-2-(4-phenyl-piperazin-1-yl)-ethyl]-amide), and other CGRP receptor antagonists (see, Arulmani et ah, 2004, Eur J Pharmacol 500:315-330 for review)

The present medicaments can optionally be combined with one or more opioids, e.g. buprenorphine, butorphanol, codeine, fentanyl, hydrocodone, hydromorphone, methadone, morphine, oxycodone, or propoxyphene.

The present medicaments can optionally be combined with one or more glutamate antagonists, e.g. NMDA antagonists, AMPA antagonists, or kainate receptor antagonists. Examplary glutamate receptor antagonists include D-AP5 (D(−)-2-amino-5-phosphonopentanoate), CGS19755 (4-phosphonomethyl-2-piperidine carboxylic acid), CGP37849 (D,L-(E)-2-amino-4-methylphosphono-3-pentanoic acid), LY233053 (cis-(±)-4-(2H-tetrazol-5-yl)methyl-piperidine-2-carboxylic acid), AIDA (1-aminoindan-1,5(RS)-dicarboxylic acid), (S)-(+)-CBPG ((s)-(+)-2-(3′-carboxy-bicyclo(1.1.1.)pentyl)glycine), CPCCOEt (cyclopropan(b)chromen-1a-carboxylate), EGLU ((s)-(α)-ethylglutamate), LY307452 (2s,4s-2-amino-4-(4,4-diphenylbut-1-yl)pentan-1,5-dioc acid) LY341495 (2s-2-amino-2-(1s,2s-2-carboxy-cyclopropan-1-yl)-3-(xanth-9-yl)propanoic acid), PCCG-4 (2s,1's,2's,3′R)-2-(2′-carboxy-3′-phenylcyclopropyl)glycine), 4-CPG (4-carboxyphenylglycine), memantine, and amantadine.

The present medicaments can optionally be combined with one or more NMDA antagonist, e.g., L-glutamate derivatives, tetrahydroquinoline, imidazoloquinoxalinone, isatine, fused cycloalkylquinoxalinediones, quinoxaline, spermine, a 4-hydroxy-3-nitro-1,2-dihydroquinolon-2-one derivative, an indole derivative, a benzo-thiadiazine dioxide derivative, an indeno(1,2-b)pyrazin-3-one or corresponding 2,3-dione, a quinoline derivative, an ethyl (phenylcarbamoyl)ethenyl)dichloroindole carboxylate, a thienopyrazine 2,3-dione derivative, a 2-(2,3-dicarboxycyclopropyl) glycine, a 2-amino-3-substituted phenyl propionic acid derivative, 1-carboxyalkylquinoxaline-2,3(1H,4H) dione derivative, a thienyl-glycine derivative, a benzo-fused azacyclic compounds, an indole derivatives, a tricyclic quinoxaline-diene derivative, a 3-hydroxy anthranilic acid and salts, a decahydroisoquinoline compound, a tri- or terta-substituted guanidine derivatives, a D- or L-tryptophan derivative, a tetrazolyl(alkyl)-cyclohexykaminoacid derivative, an octahydrophenanthrene derivative, a benzomorphan compound, a piperazinyl or piperidinyl-alkyl substituted isoxazole derivative, a decahydroisoquinoline-3-carboxylic ester or amide preparation, a compounds based on Conantokin-G peptide, a 3-heterocyclykalkyl-benzopyran-2-one derivative, a phosphono-alkyl imidazo-pyrimidine carboxylic acid derivative, amantidine, memantine, rimantidine, a histogranin peptide or analogue, a nitrobenzoic acid derivative, e.g. 44(2-methoxycarbonyl-4-nitrophenyl)methyl)piperazine carboxylic acid, a diamine derivative with selective sigma receptor affinity, remacemide (2-amino-N-(1,2-diphenyl-1-methylethyl)acetamide), a phosphono-alkylidene- or phosphono-alkoxyimino-piperidine acid, a benzothiadiazine carboxylic acid derivative, a dihydro-benzothiadiazine dioxide carboxylic acid derivative, a 4-hydroxy 2 (H) pyrrolone derivative, a quinoxaline derivative, a tetrahydro-imidazo (1,2-a) pyrimidines or its salt, a alpha-amino acid, a 4-hydroxy-pyrrolo[1,2-b]pyridazin-2(1H)-one derivative, a nitroquinolone derivative, a 3-aryl-substd 2(1H)quinolone, a 2(1H)-quinolone, a phosphonic acid quinoline-2-carboxylic acid derivative, its per hydro quinoline derivative or salt, a benzimidazole(s) carrying 2 acidic groups, an N,N′-disubstituted guanidine derivative, a tricyclic quinoxaline dione, a 2-(2,3-dicarboxycyclopropyl) glycine stereoisomer, pregnenolone sulphate or one of its derivative, an isatine derivative, a 3-amino-indolyl-derivative, 2-phenyl-1,3-propanediol dicarbamate (felbamate), a benzomorphan derivative, a dihydrothienopyridine derivative, an enantiomer of (aminophenyl)-heteroaryl ethylamine, a pyridazine-dione derivative, a 2H-1-benzopyran-2-one compound, a 4-sulphonylamino-quinoline derivative, a R(plusy3-amino-1-hydroxy-pyrrolidine-2-one, a 2-carboxy indole, a substd. imino-methano dibenzo (A,D) cycloheptene derivative, an indole-hydrazone, a piperazine derivative, a 4,6-disubstituted tryptophan and kynurenine derivative, a fluorenamine compound, a diketo-pyrido pyrazine derivative or its salts, a 2-amino-3,4-dioxo-1-cyclobutene derivative, a 2-acyl-amido derivative of 3,4-dihydro-3-oxo-quinoxaline, a benzimidazole phosphono-aminoacid derivative, a quinoxaline phosphono-aminoacid derivative, a piperazine, piperidine or pyrrolidone derivative, ist salts and isomeric forms including stercoisomers, δ 4-hydroxy-2(1H)-quinolinone derivative, ist salts and prodrugs, a fused pyrazine derivative, a 2-phenyl or 2-thienyl-(2)-piperidine derivative, a 3-amido or 3-sulphamido-indolyl derivative, a 3-aryl-4-hydroxy-2-(1H)-quinolone derivative, a 2-heterocyclyk2-hydroxy-ethylamine derivative, a 1-aryl-2-aminomethylpyrrolidine, its optical isomers and acid-addn. salts, a 4,6-dihalo indole2-carboxylic acid derivative, a cyclic aminohydroxamate derivative, a tetracyclic amine derivative, a 2,4-dioxo-1,2,3,4-tetrahydroquinoline derivative, a 2,4-dioxo-1,2,3,4-tetrahydroquinoline derivative, a 3-phosphonopiperidine and p-pyrrolidine derivative, a benzothieno (2,3-B)-pyrazine-2,3-(1H,4H)-dione, a spiro dibenzosuberane derivative, a benzomorphan derivative, a preparation of 3,4-disubstituted 2-isoxazoline(s) and isoxazoles(s), a 3-indolyl thio-acetate derivative, an arginine-derived nitric oxide biosynthesis inhibitor, a dicyclic amine derivative, a spiroisoindole derivative, an imidazo(1,2-A)-pyridinylalkyl compound, a 1,2,3,4-tetrahydro-9H-pyrido indole or benzothiophene derivative, an indole-2,3-dione-3-oxime derivative, a 1-aryl-2-(aminomethyl)cyclopropanecarboxamide derivative, a 4-phosphono-2-amino-alkenoic acid derivative, a naphthopyran derivative, a beta-ketone, a beta oxime or beta hydrazine phosphonate, a topa quinone aminoacid, kynurenic acid or a derivative, a quinoline- or thienopyridine-carboxylic acid derivative, a 10,5⁻(imino-methano)-10,11-dihydro-5H-dibenzo(A,D)cyclohept ene or a derivative, a bicyclic amino-hydroxamate derivative, an indole-2-carboxylic acid derivative, a substituted adamantane derivative, a benzobicycloalkane derivative, a 2,4-disubstituted-1,2,3,4-tetrahydro-quinoline derivative, a dihydro-alkyl-substituted (immunomethano)-5H-dibenzo-cycloheptene, an aryl cyclohexylamine, an N-substd. benzobicycloalkane amine, an isoquinoline phosphonate derivative, an N,N′-disubstd.-guanidine compound, a phosphonopropenyl piperidine carboxylic acid compound, (2R,3S,4S)-alpha-carboxycyclo-propyl-glycine, a pyrrolidine derivative, a dihydroxy-fused heterocyclyl quinoxaline derivative, a hydrogenated derivative of MK801 and analogues, a 5-substd. 10,11-dihydro 5H-dibenzo (A,D) cycloheptene 5,10-imine, an 11-Exo-hydroxy MK 801 preparation including electrochemical cyclisation step to form 5,10-imine bridge in 5-methyl 5-oxyamino 5H-dibenzo (A,D) cycloheptene, a tetrahydro-isoquinoline or 2-benzazepine derivative, an N-3-phenylpropionyl-substd. spermine or related polyamine derivative, a 4a-amino-fluorene compound or a heterocyclic analogue, a cyclooctane-imine derivative, a R-3-amino-1-hydroxy pyrrolidin-2-one or methionine hydroxamate, a 10,11-dihydro-5H-dibenzo-cyclohepten-5,10-imine compound, a polyhydro-10,11-dihydro-5H-benzo(a,d)cyclohepten-5,10 imine derivative, a 4-oxo-1,4-dihydroquinoline compound with 2-acidic groups, a heterocyclykalkene-phosphonic acid compound, a phosphono gp-containing pyridine 2-carboxylic acid, an alpha-amino-alpha-(3-alkylphenyl)alkyl ethanoic acid, its esters or amides, a 10,11-dihydro-5H-dibenzo-A,D-cyclohepten-5,10-imine compound, a phosphorus containing unsaturated amino acid or its salts, a 5 Substd.-1-, 11-dihydro-5H-dibenzo-cyclohepten-5,10-imine or analogue, a heterocyclic phosphonic acid derivative or its salt, a substituted 4-(aminocarbonyl-amino)quinoline derivative, a tricyclic quinoxaline derivative, a butyryltyrosine spermine or one of its analogue, a tri- or tetra-substituted guanidine, a quinoxalinylalkyl-aminoalkane phosphonic acid derivative, a 2-(aminophenyl)-3-(2-carboxy-indol-3-ylypropenoic acid derivative, a 6-piperidinylpropiony-2(3H)-benzoxazolone derivative, 6-(3-[4-(4-fluorobenzyl)piperidin-1-yl]propionyl)-3H-benzoxazol-2-one or one of its salts, an imidazo[1,2-a]pyridine compound, a tetrahydroquinoline derivative or one of its salts, a 2-methyl-5,8-substituted 2,3,4,5-tetra- or 2,3A4a,5,9b-hexahydro-1H-pyrido[4,3-b]indole, a 3-aminoindolyl compound, a 6-pyrrolyl-quinoxaline-2,3-dione derivative, an imidazoly(mercaptoalkyl)-quinoxaline-dione compound, a 3-amidoindolyl derivative, a heterocyclyl-imidazolo-quinoxalinone compound, a naphthyl-substituted alpha-amino acid derivative, a 5-heteroaryl-2,3-quinoxaline-dione derivative, a quinoxaline derivative, a 5H, 10H-imidazo indeno 4-pyrazinone derivative, a hydroxy-(aryl-substituted phenyl)-quinolone compound, an imidazo indolo pyrazinone derivative, a ((phenyl-amino)-(m) ethylypyridine derivative, a tetrahydro-isoquinoline derivative, a 4-substituted piperidine analogue, a 2-substituted piperidine derivative, a tri- or tetra-substituted guanidine derivative, a 3-Hydroxy-4-imidazolidinone, a 3-aminoquinoxalin-2-one derivative, rapamycin or a derivative e.g. 1,3-Diels Alder adduct with phenyl-triazoline-dione, 1-amino-1-cyclobutanecarboxylic acid, a thiamorphinan derivative, a pyrido[4,3-b]indole derivative, 4-phenyl carbamoyl methylene tetrahydro quinoline-2-carboxylic acid or a derivative thereof, (3R,4SY3-(4-(4-fluorophenyl)-4-hydroxy-piperidin-1-yl)-chroman-4,7-dio a phenol derivative, an indeno-pyrazin-4-one, a 2,3-dioxo-1,2,4,5-tetrahydro-quinoxalinyl derivative, a 45-bridged quinoxalinedione or quinolone, (1S,2S)-1-(4-hydroxyphenyl)2-(4-hydroxy 4-phenyl piperidin-1-yl) I-propanol methane sulphonate trihydrate, a 4-sulphanimide-quinoline derivative, a methanobenzocyclodecen-13-amine compound, a derivatives of pregnenolone sulphate, a quinoxalinyl-(alkane,alkene, or alkyne)-phosphonic acid or one of ist esters, a diarylalkylamine related to spider and wasp venom toxins, a piperazine R-alpha-carboxylic acid derivative, an imidazo-indeno-pyrazin-4-one derivative, a pyridazino-quinoline derivative, a 1-substituted, or 1,3-di-substituted, 1,3-diaryl-guanidine compound, an aza-cycloalkykfused quinoxaline-dione, a 3-substd, 2-carboxy-indole derivative or intermediate, a (2R)—N-trityl-4-oxo-5-(dimethyl phosphono)-nor-valinate ester, a kynurenic acid derivative, an indole carboxylic acid derivative, a 6-(tetrazolyl or isoxazolyl-decahydroisoquinoline-3-carboxylic acid derivative, a phenyl- or pyridinyl-thieno-pyridinone derivative, a fused cycloalkylquinoxaline-dione derivative, a pyridazino-quinoline derivative, a 1-Alpha-amino-3-biphenyl-propanoic acid derivative, a 3-(Indo)-3-yl) propenoic acid derivative, a spiro-heterocycle-midazo-indeno-pyrazine-4-one derivative, a 2-heterocyclyk3-indolylpropenoic acid derivative, a piperidinoalkyl heterocyclic ketone or alcohol compound, a pyrrolyl-tetrahydro-benzoquinoxaline-dione derivative, a 7-imidazolyl or dialkylamino,tetrahydroquinoxaline dione compound, a dibenzocycloheptene, a quinoxaline derivative, an aryl-thio-quinoxaline derivative, a heterocyclic substd. imidazolo-quinoxaline derivative, a 1,4-dihydro-quinoxaline-2,3-dione derivative, an oxa- or thia-aliphatically bridged quinoxaline derivative, an aza-aliphatically bridged quinoxaline-2,3-dione compound, a 3-amido- or 3-sulphamido-indole compound, a 3,5-disubstd. phenyl-naphthalene derivative, an imidazo (1,2-a)indeno (1,2-e) pyrazine-2-carboxylic acid derivative, a 3-phenyl-fused ring pyridine-dione derivative, a 2-phenyl-pyridazino-indole-dione derivative, a 4,6-disubstd. kynurenine compound, a phosphono derivative of imidazo[1,2-a]pyrimidine-2-carboxamide, a tetrahydro-quinoxaline-dione derivative with N-(alkyl)carbonyl-amino- or ureido group, a tryptophan derivative, a hetero-aliphatic or hetero-araliphatic substd. quinolone derivative, an imidazo-pyridine dicarboxylic acid derivative, a composition containing pyrazolo-quinoline derivatives, an ethanodihydrobenzoquinolizinium salt, an oxopyridinylquinoxaline derivative, an indeno-triazolo-pyrazin-4-one derivative, an imidazo-indeno-pyrazinone derivative, an imidazo-indeno-pyrazin-4-one derivative, an imidazo[1,2-a]pyrazine-4-one derivative, a 5H-indeno-pyrazine-2,3-dione derivative, a phenyl-aminoalkyl-cyclopropane N,N-diethyl carboxamide compound, a dexanabinol derivative, a substituted chroman derivative, a sulphonamide quinazoline-2-4-dione compound, a 6- and 8-aza-, and 6,8-diaza-1,4-dihydro-quinoxaline-2,3-dione derivative, a substituted quinoline derivative, a tetrazolylalkyl cyclohexyl aminoalkanoic acid, a tricyclic indole 2-carboxylic acid derivative, a 6-substd-7H-imidazo-8-pyrazinone derivative, a quinoxaline dione derivative or one of its radiolabelled compounds, a tricyclic pyridazinopyridine derivative, an N-substituted heterocyclylidenemethyl-indole carboxylic acid derivative, a 3-aza-8-substituted-bicyclo(3.3.0)octane-2-carboxylic acid derivative, an ethano-heterocyclo-isoquinolinium salt, a phenyl alkanolamine derivative, a dihydrobenzothiadiazinedioxide carboxylic acid derivative, a methyl-butenylmethyl(hydroxy-propyl)carbazoledione, an imidazo pyrazinone derivative, an imidazo-(1,2-a)pyrazine-4-one, a benzazepine-dione derivative, disulfuram, a 3-(indo13-yl)-propenoic acid derivative, a 1,2,3,4-tetrahydro-quinoline-2,3,4-trione-3 or 4-oxime compound, a peptide antagonist at NMDA receptors, a 2-amino-2-phenyl(alkylyacetic acid derivative, 6-halo-tryptophan or a 4-halo-kynurenine, a 6-tetrazolyl or isoxazoly-decahydro-isoquinoline-3-carboxylic acid derivative, an an imidazolylbenzene or salts thereof.

The present medicaments can optionally be combined with one or more AMPA antagonists, e.g., L-glutamate derivatives, amino alkanoic acid derivatives, α-amino-3-hydroxy-5-methyl-4-isoxazolepropionate derivatives, acetyl-aminophenyl-dihydro-methyl-methyl-dioxolobenzodiazepine, acid amide derivatives, amino-phenyl-acetic acid, 2,3-benzodiazepin-4-one, alkoxy-phenyl-benzodiazepine, amino- or desamino 2,3-benzodiazepine, benzothiadiazine, α-carboline-3-carboxylic acid, fused cycloalkylquinoxalinediones, decahydroisoquinoline, 4-hydroxypyrrolone, 4-hydroxy-pyrrolo-pyridazinone, imidazo-pyrazinone, imidazolo-quinoxalinone, indeno-pyrazine-carboxylic acid, indeno-pyrazinone, indoloneoxime, indolo-pyrazinone, isatine, isatinoxime, oxadiazole, phenyl-azolophthalazine, phenylpyridazino-indole-1,4-dione, quinoline, quinolinone, quinoxaline, quinoxalinedione, quinazolinone, quinolone, nitroquinolone, and sulphamate derivatives.

The present medicaments can optionally be combined with one or more kainate receptor antagonists, e.g., L-glutamate derivatives, kainic acid derivatives, acid amide derivatives, aminoalkanoic acid derivatives, aminophenyl(alkyl)acetic acid derivatives, fused cycloalkylquinoxalinediones, quinoxalinedione, imidazolo-quinoxalinone, isatine, phenylazolophthalazine, pyridothiazines, 4-phosphonoalkyl-quinolinone, quinolinone, quinazoline, quinazolinedione, quinoxalinedione, and sulphamate derivatives.

The present medicaments can optionally be combined with one or more inhibitors of glutamate release, e.g., lamotrigine, BW1003C87, riluzole, isoguvacine, muscimol, THIP, piperidine-4-sulphonic acid, flunitrazepam, zolpidem, abecarnil, ZK93423, L-baclofen, CGP27492, piracetam, progabide, and CGP35024.

The present medicaments can optionally be combined with one or more glutamate reuptake promoters, e.g. zonisamide.

The present medicaments can optionally be combined with one or more combined serotonin/noradrenaline reuptake inhibitors, e.g., venlafaxine, milnacipran, duloxetine, pregabalin, LY248686, and Strattera.

The present medicaments can optionally be combined with one or more tricyclic antidepressants, e.g., amitriptyline, amitriptylinoxide, butriptyline, clomipramine, demexiptiline, desipramine, dibenzepin, dimetacrine, dosulepin/dothiepin, doxepin, imipramine, imipraminoxide, lofepramine, melitracen, metapramine, nitroxazepine, nortriptyline, noxiptiline, pipofezine, propizepine, protriptyline, and quinupramine.

The present medicaments can optionally be combined with one or more tetracylic antidepressants, e.g., amitriptyline, amitriptylinoxide, butriptyline, clomipramine, demexiptiline, desipramine, dibenzepin, dimetacrine, dosulepin/dothiepin, doxepin, imipramine, imipraminoxide, lofepramine, melitracen, metapramine, nitroxazepine, nortriptyline, noxiptiline, pipofezine, propizepine, protriptyline, or quinupramine.

The present medicaments can optionally be combined with one or more dopamine antagonist, e.g., domperidone, droperidol, haloperidol, chlorpromazine, promethazine, prochlorperazine, alizapride, or prochlorperazine.

The present medicaments can optionally be combined with one or more nk1 receptor antagonists, e.g., aprepitant or casopitant.

The present medicaments can optionally be combined with one or more antihistamine, e.g., cyclizine, diphenhydramine dimenhydrinate, meclizine, promethazine, or hydroxyzine.

The present medicaments can optionally be combined with one or more benzodiazepines, e.g., midazolam or lorazepam.

The present medicaments can optionally be combined with one or more anticholinergics, e.g., hyoscine.

The present medicaments can optionally be combined with one or more or steroids, e.g, dexamethasone.

Examplary Medicaments

By way of example, any of the formulations set forth in Table 1 can usefully be prepared to provide the PK profile of any of PK Profile 1 through PK Profile 19.

TABLE 1
Examplary Medicaments

| | | % of | | |
|---|---|---|---|---|
| | | cannabinoid | | |
| formu- | Total | in the | IR Dosage | |
| lation | Cannabinoid | IR Dosage | Compart- | DR Dosage |
| # | amount (mg) | compartment | ment | Compartment |
| 1 | 1 | 25, 40, | oil | SEDDS DR |
| | | 50, 60, 75, | | |
| | | and 80 | | |
| 2 | 2.5 | 25, 40, | oil | SEDDS DR |
| | | 50, 60, 75, | | |
| | | and 80 | | |
| 3 | 5 | 25, 40, | oil | SEDDS DR |
| | | 50, 60, 75, | | |
| | | and 80 | | |
| 4 | 10 | 25, 40, | oil | SEDDS DR |
| | | 50, 60, 75, | | |
| | | and 80 | | |
| 5 | 20 | 25, 40, | oil | SEDDS DR |
| | | 50, 60, 75, | | |
| | | and 80 | | |
| 6 | 1 | 25, 40, | oil | DR compartment |
| | | 50, 60, 75, | | from Example 10 |
| | | and 80 | | |
| 7 | 2.5 | 25, 40, | oil | DR compartment |
| | | 50, 60, 75, | | from Example 10 |
| | | and 80 | | |
| 8 | 5 | 25, 40, | oil | DR compartment |
| | | 50, 60, 75, | | from Example 10 |
| | | and 80 | | |
| 9 | 10 | 25, 40, | oil | DR compartment |
| | | 50, 60, 75, | | from Example 10 |
| | | and 80 | | |
| 10 | 20 | 25, 40, | oil | DR compartment |
| | | 50, 60, 75, | | from Example 10 |
| | | and 80 | | |
| 11 | 1 | 25, 40, | oil | oil or aqueous |
| | | 50, 60, 75, | | liquid-filled |
| | | and 80 | | capsule(s) with |
| | | | | DR coating |
| 12 | 2.5 | 25, 40, | oil | oil or aqueous |
| | | 50, 60, 75, | | liquid-filled |
| | | and 80 | | capsule(s) with |
| | | | | DR coating |
| 13 | 5 | 25, 40, | oil | oil or aqueous |
| | | 50, 60, 75, | | liquid-filled |
| | | and 80 | | capsule(s) with |
| | | | | DR coating |
| 14 | 10 | 25, 40, | oil | oil or aqueous |
| | | 50, 60, 75, | | liquid-filled |
| | | and 80 | | capsule(s) with |
| | | | | DR coating |
| 15 | 20 | 25, 40, | oil | oil or aqueous |
| | | 50, 60, 75, | | liquid-filled |
| | | and 80 | | capsule(s) with |
| | | | | DR coating |
| 16 | 1 | 25, 40, | oil | dried solid with |
| | | 50, 60, 75, | | DR coating |
| | | and 80 | | |
| 17 | 2.5 | 25, 40, | oil | dried solid with |
| | | 50, 60, 75, | | DR coating |
| | | and 80 | | |
| 18 | 5 | 25, 40, | oil | dried solid with |
| | | 50, 60, 75, | | DR coating |
| | | and 80 | | |
| 19 | 10 | 25, 40, | oil | dried solid with |
| | | 50, 60, 75, | | DR coating |
| | | and 80 | | |
| 20 | 20 | 25, 40, | oil | dried solid with |
| | | 50, 60, 75, | | DR coating |
| | | and 80 | | |
| 21 | 1 | 25, 40, | crystalline | crystalline form |
| | | 50, 60, 75, | form | with DR coating |
| | | and 80 | | |
| 22 | 2.5 | 25, 40, | crystalline | crystalline form |
| | | 50, 60, 75, | form | with DR coating |
| | | and 80 | | |
| 23 | 5 | 25, 40, | crystalline | crystalline form |
| | | 50, 60, 75, | form | with DR coating |
| | | and 80 | | |
| 24 | 10 | 25, 40, | crystalline | crystalline form |
| | | 50, 60, 75, | form | with DR coating |
| | | and 80 | | |
| 25 | 20 | 25, 40, | crystalline | crystalline form |
| | | 50, 60, 75, | form | with DR coating |
| | | and 80 | | |
| 26 | 1 | 25, 40, | SEDDS IR | SEDDS DR |
| | | 50, 60, 75, | | |
| | | and 80 | | |
| 27 | 2.5 | 25, 40, | SEDDS IR | SEDDS DR |
| | | 50, 60, 75, | | |
| | | and 80 | | |
| 28 | 5 | 25, 40, | SEDDS IR | SEDDS DR |
| | | 50, 60, 75, | | |
| | | and 80 | | |
| 29 | 10 | 25, 40, | SEDDS IR | SEDDS DR |
| | | 50, 60, 75, | | |
| | | and 80 | | |
| 30 | 20 | 25, 40, | SEDDS IR | SEDDS DR |
| | | 50, 60, 75, | | |
| | | and 80 | | |
| 31 | 1 | 25, 40, | crystalline | DR compartment |
| | | 50, 60, 75, | form | from Example 10 |
| | | and 80 | | |
| 32 | 2.5 | 25, 40, | crystalline | DR compartment |
| | | 50, 60, 75, | form | from Example 10 |
| | | and 80 | | |
| 33 | 5 | 25, 40, | crystalline | DR compartment |
| | | 50, 60, 75, | form | from Example 10 |
| | | and 80 | | |
| 34 | 10 | 25, 40, | crystalline | DR compartment |
| | | 50, 60, 75, | form | from Example 10 |
| | | and 80 | | |
| 35 | 20 | 25, 40, | crystalline | DR compartment |
| | | 50, 60, 75, | form | from Example 10 |
| | | and 80 | | |
| 36 | 1 | 25, 40, | oil | crystalline form |
| | | 50, 60, 75, | | with DR coating |
| | | and 80 | | |
| 37 | 2.5 | 25, 40, | oil | crystalline form |
| | | 50, 60, 75, | | with DR coating |
| | | and 80 | | |
| 38 | 5 | 25, 40, | oil | crystalline form |
| | | 50, 60, 75, | | with DR coating |
| | | and 80 | | |
| 39 | 10 | 25, 40, | oil | crystalline form |
| | | 50, 60, 75, | | with DR coating |
| | | and 80 | | |
| 40 | 20 | 25, 40, | oil | crystalline form |
| | | 50, 60, 75, | | with DR coating |
| | | and 80 | | |
| 41 | 1 | 25, 40, | aqueous | solid with DR |
| | | 50, 60, 75, | liquid | coating |
| | | and 80 | | |
| 42 | 2.5 | 25, 40, | aqueous | solid with DR |
| | | 50, 60, 75, | liquid | coating |
| | | and 80 | | |
| 43 | 5 | 25, 40, | aqueous | solid with DR |
| | | 50, 60, 75, | liquid | coating |
| | | and 80 | | |
| 44 | 10 | 25, 40, | aqueous | solid with DR |
| | | 50, 60, 75, | liquid | coating |
| | | and 80 | | |
| 45 | 20 | 25, 40, | aqueous | solid with DR |
| | | 50, 60, 75, | liquid | coating |
| | | and 80 | | |
| 46 | 1 | 25, 40, | oil | osmotic |
| | | 50, 60, 75, | | |
| | | and 80 | | |
| 47 | 2.5 | 25, 40, | oil | osmotic |
| | | 50, 60, 75, | | |
| | | and 80 | | |
| 48 | 5 | 25, 40, | oil | osmotic |
| | | 50, 60, 75, | | |
| | | and 80 | | |
| 49 | 10 | 25, 40, | oil | osmotic |
| | | 50, 60, 75, | | |
| | | and 80 | | |
| 50 | 20 | 25, 40, | oil | osmotic |
| | | 50, 60, 75, | | |
| | | and 80 | | |
| 51 | 1 | 25, 40, | aqueous | osmotic |
| | | 50, 60, 75, | liquid | |
| | | and 80 | | |
| 52 | 2.5 | 25, 40, | aqueous | osmotic |
| | | 50, 60, 75, | liquid | |
| | | and 80 | | |
| 53 | 5 | 25, 40, | aqueous | osmotic |
| | | 50, 60, 75, | liquid | |
| | | and 80 | | |
| 54 | 10 | 25, 40, | aqueous | osmotic |
| | | 50, 60, 75, | liquid | |
| | | and 80 | | |
| 55 | 20 | 25, 40, | aqueous | osmotic |
| | | 50, 60, 75, | liquid | |
| | | and 80 | | |
| 56 | 1 | 25, 40, | Adsorbate | Adsorbate solid |
| | | 50, 60, 75, | solid | coated with a DR |
| | | and 80 | | Coating |
| 57 | 2.5 | 25, 40, | Adsorbate | Adsorbate solid |
| | | 50, 60, 75, | solid | coated with a DR |
| | | and 80 | | Coating |
| 58 | 5 | 25, 40, | Adsorbate | Adsorbate solid |
| | | 50, 60, 75, | solid | coated with a DR |
| | | and 80 | | Coating |
| 59 | 10 | 25, 40, | Adsorbate | Adsorbate solid |
| | | 50, 60, 75, | solid | coated with a DR |
| | | and 80 | | Coating |
| 60 | 20 | 25, 40, | Adsorbate | Adsorbate solid |
| | | 50, 60, 75, | solid | coated with a DR |
| | | and 80 | | Coating |
| 61 | 1 | 25, 40, | Complexation | Complexation |
| | | 50, 60, 75, | Solid | solid coated with |
| | | and 80 | | a DR coating |
| 62 | 2.5 | 25, 40, | Complexation | Complexation |
| | | 50, 60, 75, | Solid | solid coated with |
| | | and 80 | | a DR coating |
| 63 | 5 | 25, 40, | Complexation | Complexation |
| | | 50, 60, 75, | Solid | solid coated with |
| | | and 80 | | a DR coating |
| 64 | 10 | 25, 40, | Complexation | Complexation |
| | | 50, 60, 75, | Solid | solid coated with |
| | | and 80 | | a DR coating |
| 65 | 20 | 25, 40, | Complexation | Complexation |
| | | 50, 60, 75, | Solid | solid coated with |
| | | and 80 | | a DR coating |
| 66 | 1 | 25, 40, | Adsorbate | Adsorbate solid |
| | | 50, 60, 75, | solid | in a DR matrix |
| | | and 80 | | |
| 67 | 2.5 | 25, 40, | Adsorbate | Adsorbate solid |
| | | 50, 60, 75, | solid | in a DR matrix |
| | | and 80 | | |
| 68 | 5 | 25, 40, | Adsorbate | Adsorbate solid |
| | | 50, 60, 75, | solid | in a DR matrix |
| | | and 80 | | |
| 69 | 10 | 25, 40, | Adsorbate | Adsorbate solid |
| | | 50, 60, 75, | solid | in a DR matrix |
| | | and 80 | | |
| 70 | 20 | 25, 40, | Adsorbate | Adsorbate solid |
| | | 50, 60, 75, | solid | in a DR matrix |
| | | and 80 | | |
| 71 | 1 | 25, 40, | Complexation | Complexation |
| | | 50, 60, 75, | Solid | solid in a DR |
| | | and 80 | | matrix |
| 72 | 2.5 | 25, 40, | Complexation | Complexation |
| | | 50, 60, 75, | Solid | solid in a DR |
| | | and 80 | | matrix |
| 73 | 5 | 25, 40, | Complexation | Complexation |
| | | 50, 60, 75, | Solid | solid in a DR |
| | | and 80 | | matrix |
| 74 | 10 | 25, 40, | Complexation | Complexation |
| | | 50, 60, 75, | Solid | solid in a DR |
| | | and 80 | | matrix |
| 75 | 20 | 25, 40, | Complexation | Complexation |
| | | 50, 60, 75, | Solid | solid in a DR |
| | | and 80 | | matrix |
| 76 | | | | |
| 77 | | | | |
| 78 | | | | |
| 79 | | | | |
| 80 | | | | |
| 81 | | | | |
| 82 | | | | |

In one embodiment, a medicament comprises a formulation comprising components set forth in Table 1, except that the total cannabinoid amount is +/−50% (e.g. +/−30% or +/−20%) of that listed in Table 1.

Therapeutic Window

Medicaments of the present invention provide a therapeutic window that is a longer window than provided by an immediate release medicament (e.g. as compared to a Marinol capsule containing an equivalent amount of the cannabinoid). Examples of useful therapeutic windows provided by the present medicaments are about 4 to about 12 hours. Optionally, useful therapeutic windows provided here are about 6 to about 10 hours, about 6 to about 9 hours, about 6 to about 8 hours, about 7 to about 8 hours, or about 7 to about 9 hours.

Optionally, within an hour or two following the end of the therapeutic window, the plasma levels are reduced below a level that can result in undesired side effects such as impeded cognitive and/or psychomotor performance, tachycardia, hypotension, or impaired learning and memory.

Dosing

Compositions of the present invention comprise cannabinoids in an amount of about 0.1 mg to about 75 mg or optionally about any of the following amounts (in mg): 0.5 to 50, 0.5 to 25, 0.5 to 20, 0.5 to 10, 1 to 15, 1 to 5, 1 to 2.5, 2.5 to 50, 2.5 to 20.

The unexpected results from the clinical studies reported here teach useful doses for populations and for individuals. However, the skilled artisan will readily recognize from these studies that the therapeutic level and side-effect producing levels of plasma THC can vary within individuals. For example, while a 10 mg THC medicament of the present invention typically will provide a side-effect sparing efficacy for an extended treatment window, certain individuals will have a higher threshold for both therapeutic efficacy and for side effects. The same is true for certain individuals who will have a lower threshold for efficacy and for side effects. Therefore, there is a remarkable and unexpected utility of the present medicaments containing a cannabinoid in the full range of about 0.1 mg to about 75 mg.

In one embodiment, the invention provides a method of treating apnea comprising administering less than about 20 mg of cannabinoid during a therapeutic window taught herein.

Titration

The skilled artisan will recognize (with the teachings of this invention) that “optimal” dosing of an individual is determined by evaluating, among other factors, efficacy and safety (i.e. the “therapeutic profile”). Through inventor insight, it is apparent that subjects with certain cannabinoid-sensitive disorders may demonstrate an increased-responsiveness to medicaments of the present invention upon an extended treatment period (e.g. about one week or longer, or about 2 weeks or longer, or about one month or longer, or about six months or longer). Taken together, in one embodiment, the present invention is a titration kit and titration methods for determining optimal dose.

Subjects are initially administered a low dose of the medicament for a treatment period. At the completion of the treatment period, the dose in the medicament is increased or the number of medicament units is increased (a “step-up”) for an additional treatment period. This “escalation” cycle can be repeated multiple times until 1) optimal clinical benefit is achieved, 2) clinically relevant side effects become apparent, or 3) until the maximum dose generally considered safe is administered.

Optionally, treatment-related side effects (or trial related adverse events) are evaluated during treatment periods. Relevant evaluations include mental alertness, emotional health, quality of life, sleepiness, etc.

Optionally, a clinically-relevant metric(s) of the disorder or condition being treated is assessed during each treatment period. Methods are readily known for quantifying or assessing apnea, pain, spasms, etc.

Optionally, an initial “low dose” THC medicament of the present invention can contain about any mg amounts of a cannabinoid, e.g. 0.1, 0.5, 1, 2, 5, 10, 20, or 50 (mg).

Optionally, a treatment period for each dose is about 1 to about 10 days or about 5 to about 10 days, or longer than 10 days.

A typical step-up dose increase is any percent of about 10, 20, 25, 33, 50, 100, 200, or 400(%).

As a result of the titration study, an empirically determined dose that is well tolerated (minimal or no significant side effects) and optimally effective is selected. This selected dose is administered for another period (e.g. 1 or more days or more than 1 week or more than 1 month.

Optionally, the subject is administered a “step down” lower dose medicament (e.g. about 50% to about 75% or about 20% to about 50% of the previous dose). A clinically relevant metric of efficacy and side effects are assessed. If therapeutic efficacy is not diminished (over the previous dose), the subject can optionally be administered a dose with a further reduction (i.e. a second or subsequent step-down).

In one embodiment, the subject has sleep apnea and is administered a 0.1 or 0.5 mg THC medicament of the present invention (e.g. 0.5, 1, 1.5, or 2 hrs before anticipated sleep time) for a treatment period. During this treatment period, overnight PSG is optionally performed. If the patient tolerates this dose (e.g. minimal treatment related side effects), a step-up dose is administered daily for another treatment period. Therapeutic profile is assessed, and a subsequent escalation is performed until clinically-relevant side effects are observed or maximal safe dose is administered.

A step-down titration is optionally performed and evaluated.

In one embodiment of the present invention, a kit is provided that contains an appropriate number of one or more doses of a medicament (otherwise of the same formulation). The kit optionally contains patient instructions. Optionally, the doses are in a device that compartmentalizes the daily doses (e.g. a blisterpack).

Safety

Unexpectedly, it has been discovered that oral administration exemplary present medicaments maintains a therapeutic window while not resulting in a plasma levels at any time throughout the treatment window (sleep period) that increase the likelihood of side effects. Such side effect-sparing medicaments avoid one or more of the effects shown in Table 2.

Unexpectedly, it has been discovered that oral administration of examplary present medicaments maintains a therapeutic window (sleep period) while not resulting in a plasma levels at any time throughout the treatment window that increase the likelihood of side effects associated with co-administration of other prescription or over the counter medicines such as shown in Table 3.

TABLE 2
Dronabinol Side Effects

| | Side Effects |
|---|---|
| Cardiovascular: | Conjunctivitis*, hypotension*., |
| | tachycardia, hypotension |
| Digestive: | Diarrhea*, fecal incontinence. |
| Musculoskeletal: | Myalgias. |
| Nervous system: | Depression, nightmares, |
| | speech difficulties, tinnitus. |
| | impeded cognitive and/or |
| | psychomotor performance,, or |
| | impaired learning and memory |
| Skin and | Flushing*. |
| Appendages: | |
| Special senses: | Vision difficulties. |

*Incidence of events 0.3% to 1%

TABLE 3
Dronabinol Side Effects Associated with Co-administration of
Other Drugs

| Drug | Side effects |
|---|---|
| Amphetamines, cocaine, | Additive hypertension, tachycardia, possibly |
| other sympathomimetic | cardiotoxicity |
| agents | |
| Atropine, scopolamine, | other anticholinergic agents, Additive or |
| antihistamines, | super-additive tachycardia, drowsiness |
| Amitriptyline, amoxapine, | Additive tachycardia, hypertension, |
| desipramine, other tricyclic | drowsiness |
| antidepressants | |
| Barbiturates, benzo- | Additive drowsiness and CNS depression |
| diazepines, ethanol, | |
| lithium, opioids, | |
| buspirone, antihistamines, | |
| muscle relaxants, other | |
| CNS depressants | |
| Disulfiram A | reversible hypomanic reaction was reported |
| | in a 28 y/o man who smoked marijuana; |
| | confirmed by dechallenge and rechallenge |
| Fluoxetine | A 21 y/o female with depression and bulimia |
| | receiving 20 mg/day fluoxetine × 4 wks |
| | became hypomanic after smoking marijuana; |
| | symptoms resolved after 4 days |
| Antipyrine, barbiturates | Decreased clearance of these agents, |
| | presumably via competitive inhibition of |
| | metabolism |
| Theophylline | Increased theophylline metabolism reported |
| | with smoking of marijuana; effect |

Utility

The present methods and medicaments are useful for treating cannabinoid-sensitive disorders.

The present methods and medicaments are especially useful for treating apnea. Optionally, the apnea is any of obstructive sleep apnea syndrome, obstructive sleep apnea/hypopnea syndrome, upper airway resistance syndrome, apnea of prematurity, congenital central hypoventilation syndrome, obesity hypoventilation syndrome, central sleep apnea syndrome, Cheyne-Stokes respiration, and snoring. Unexpectedly, the present compositions are useful to reduce episodes of apnea, snoring, and sleep disruption, for example as demonstrated by oximetry, or polysomnogram (“PSG”), or self assessment.

In one embodiment, the administered dose and/or medicament comprises 1 mg to 75 mg (e.g. 2.5 mg to 20 mg).

At least some of the pharmacological effects of THC are exerted through the cannabinoid pathway, by interaction with cannabinoid receptors such as CB1 and/or CB2. Cannabinoid receptors are found predominantly at nerve terminals where they have a role in retrograde regulation of synaptic function and interact with at least acid (e.g. GABA, glutamate), monoamine (e.g. histamine, dopamine, serotonin, noradrenaline) purine (e.g. adenosine, ADP, ATP), peptide (e.g. somatostatin, neuropeptide Y, neurokinin, cholecystokinin), vanilloid, prostanoid, opiod and/or other pathways. For example, THCs such as delta-9-tetrahydrocannabinol act as agonists at CB1 and CB2 receptors, mimicking the effects of the naturally occurring endocannabinoids, which modulate the effects of neurotransmitters. Without being bound by theory, the inventors believe that the methods and medicaments of the present invention exert pharmacological action through modulation of one or more of these pathways. Indeed, the cannabinoid receptors are concentrated in regions of the brain that control functions associated with certain pharmacological effects of cannabinoid modulation (see Table 4).

TABLE 4
Brain Regions in Which Cannabinoid Receptors Are Abundant

| | Examplary Functions |
|---|---|
| Brain Region | Associated with Region |
| Basal ganglia | Movement control |
| Substantia nigra pars | Movement control |
| reticulata | |
| Entopeduncular nucleus | Movement control |
| Globus pallidus | Movement control |
| Putamen | Movement control |
| Cerebellum | Body movement coordination |
| Hippocampus | Learning and memory, stress |
| Cerebral cortex, especially | Higher cognitive functions |
| cingulate, | |
| frontal, and parietal regions | Higher cognitive functions |
| Nucleus accumbens | Reward center |
| Hypothalamus | Body housekeeping functions |
| | (body temperature regulation, salt |
| | and water balance, reproductive |
| | function) |
| Amygdala | Emotional response, fear |
| Spinal cord | Peripheral sensation, including |
| | pain |
| Brain stem | Sleep and arousal, temperature |
| | regulation, motor control |
| Central gray | Analgesia |
| Nucleus of the solitary tract | Visceral sensation, nausea and |
| | vomiting |

Based upon the insight of the inventors, the present methods and medicaments are surprisingly effective in treating disorders associated with the above-mentioned pathways, brain regions, or pharmacological effects, and other cannabinoid-sensitive disorders. Accordingly, the invention provides a method for treating a cannabinoid-sensitive disorder in a subject comprising administering to the subject a medicament taught herein. Optionally, the cannabinoid-sensitive disorder is a neurological disorder, pain, an appetite or wasting disorder, nausea, vomiting, a seizure disorder, a sleep disorder, breathing disorder, or a sleep-related breathing disorder. Optionally, the method further comprises administering an additional therapeutic agent for treating the disorder. Optionally, an additional therapeutic agent is included in the medicament. Optionally, the additional therapeutic agent is administered sequentially with the medicament.

In one embodiment, the present methods and medicaments are useful for treating a neurological disorder. Optionally, the neurological disorder is of the brain, spinal cord, peripheral nerves, or muscles. Optionally, the neurological disorder is a neurodegenerative disease, a neurological pain, a movement disorder, or a mood disorder.

In one embodiment, the present methods and medicaments are useful for treating a neurodegenerative disease. Optionally, the neurodegenerative disease is multiple sclerosis, Huntington's disease, or Alzheimer's disease.

In one embodiment, the present methods and medicaments are useful for treating a neurological pain. Optionally, the neurological pain is a central or peripheral neurological pain. Optionally, the neurological pain is chronic pain. Optionally, the neurological pain is associated with fibromyalgia, multiple sclerosis, spinal cord injury, or stroke.

In one embodiment, the present methods and medicaments are useful for treating a movement disorder. Optionally, the movement disorder is caused by abnormalities in the basal ganglia. Optionally, the movement disorder is a spasm disorder, muscle spasticity, seizure disorder, chorea, Huntington's disease, dystonia, basal ganglia movement disorder, Parkinson's disease, Tourette's syndrome, dyskinesia, bradykinesia, or epilepsy.

In one embodiment, the present methods and medicaments are useful for treating a mood disorder. Optionally, the movement disorder is a depressive disorder, a bipolar disorders, or anxiety.

In one embodiment, the present methods and medicaments are useful for treating pain. Optionally, the pain is neurological pain or nociceptive pain. Optionally, the pain is associated with a movement disorder, a headache, a spasm disorder, arthritis, dystonia, peripheral pain, or muscle aching. For example, treatable pain can be associated with any disorder such as fibromyalgia or multiple sclerosis.

Pain that is treatable by the present medicament includes pain associated with any of the infections caused by herpes simplex virus type 1 and type 2 and herpes zoster.

Headaches that are treatable by the present medicament include any vascular headache, e.g., migraines, cluster headache, toxic headache, and headache caused by elevated blood pressure.

Headaches that are treatable by the present medicament also include tension headaches, postcoital headaches, exertional headaches, trigeminal neuralgia, atypical trigeminal neuralgia, type 2 trigeminal neuralgia, trigeminal autonomic cephalalgias, hortons neuralgia, and histamine headaches, and headaches secondary to head or neck trauma.

In one embodiment, the present methods and medicaments are used for appetite stimulation or to treat wasting and/or depressed appetite. Optionally the wasting and/or depressed appetite is associated with HIV, chemotherapy, anorexia, or Alzheimer's disease.

In one embodiment, the present methods and medicaments are useful for treating glaucoma.

In one embodiment, the present methods and medicaments are useful for treating nausea and/or vomiting. Optionally, the nausea and/or vomiting is associated with viral/microbial illness, HIV/AIDS, cancer, chemotherapy, radiation exposure, postoperative recovery, pregnancy, motion, or poisoning.

In one embodiment, the present methods and medicaments are useful for treating a subject with a disorder selected from: anorexia, alcohol use disorders, cancer, amyotrophic lateral sclerosis, glioblastoma multiforme, glioma, increased intraocular pressure, glaucoma, inflammatory bowel disorders, arthritis, dermatitis, Rheumatoid arthritis, systemic lupus erythematosus, inflammation, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, juvenile rheumatoid arthritis, major depressive disorder, depression, brain cancer, asthma, lung cancer, chronic obstructive pulmonary disease, opioid dependence, muscle tension, posttraumatic stress disorder, and bipolar disorder.

The citations provided herein are hereby incorporated by reference for the cited subject matter.

### EXAMPLES

### Example 1

### Treatment of Apnea with Marinol

The goal of the clinical trial was to evaluate oral dosing of THC in sleep apnea patients. One objective was to assess low doses of cannabinoids for treatment of apnea. Another objective was to evaluate the therapeutic window of oral cannabinoid in the treatment of sleep-related disorders such as apnea.

The trial comprised a single-center, randomized, double-blind, placebo-controlled dose escalation study of dronabinol in 22 patients with OSAS. The study began with a 7-day baseline/PAP-washout period, with polysomnography (PSG) performed on the final night. Subjects meeting inclusion/exclusion criteria were randomized to either placebo (N=5) or dronabinol (N=17) treatment.

The study drug (active or placebo) was taken 30 min before bed for 21 days. Overnight PSG was performed on treatment nights 7, 14 and 21. The initial nightly dose was 2.5 mg and was escalated, as tolerated, to 5 mg on day 8 and to 10 mg on day 15 of treatment. A blood sample was drawn immediately after each PSG for assay of the study drug and principal metabolites.

Sleep/activity/drug logs were maintained daily throughout the study. A Stanford Sleepiness Scale (SSS) was completed every 2 waking hours for the final two days of each 7-day baseline or treatment period.

The analysis of efficacy endpoints was performed using the Efficacy Evaluable population, defined as: all subjects completing a baseline PSG who received at least one dose of study medication, who did not miss more than 3 doses during, and who completed the PSG ending the first 7-day treatment period. Safety/tolerability analyses were performed using the All-Treated population, comprising all subjects who received at least one dose of study medication. All efficacy endpoints were assessed as the change from baseline measurement of the same parameter. For example, efficacy for AHI was examined by subtracting (for each subject) the AHI measured during PSG at the end of the baseline period from the AHI measured at the end of the relevant treatment period (in both active and placebo groups). Thus a decrease in AHI with treatment is represented by a negative value for ΔAHI (change from baseline).

Results for Arousal Index (arousals per hour of sleep) are shown in Table 5. Surprisingly, these data demonstrate that oral cannabinoids provide a therapeutic benefit to sleep continuity in apnea patients, even in reduced amounts. For example, these data support treatment by administering oral doses of less than 70 mg, 60 mg, or even less than 50 mg, such as 0.1, 0.5, 1.0, or 2.5 mg-20 mg doses.

Surprisingly, these data also demonstrate that sleep apnea may be treated with orally administered cannabinoids without causing (or without substantially causing) side effects associated with certain cannabinoids and/or without causing (or without substantially causing) side effects once the subject has awoken (e.g. post treatment window).

These results, and those of the subsequent Examples, also support the applicants' invention of methods of treating sleep apnea with a reduced dose (e.g. about 0.1-about 20 mg) of an immediate release cannabinoid.

TABLE 5
Arousal Index (Arousals/Hour of Sleep)

| Parameter | 2.5 mg | 10 mg |
|---|---|---|
| Number of Subjects | 17 | 8 |
| Number of Observations | 23 | 8 |
| Mean Change with | −4.8 | −2.5 |
| treatment vs Placebo | | |
| Significance vs Placebo | 0.49 | 0.79 |

### Example 2

### Marinol for Apnea; Comparing Early and Late Treatment Windows

The study from Example 1 was further analyzed with respect to Arousal Index during the early treatment window (i.e., T₀−T₄) and the late treatment window (i.e., T₅−T₈).

TABLE 6
Arousal Index, Early and Late Treatment Window

| | 2.5 mg | 10 mg |
|---|---|---|
| 1^{st} Half of Night | | |
| Number of Subjects | 17 | 8 |
| Number of | 23 | 8 |
| Observations | | |
| Mean Change with | −20.0 | −21.9 |
| treatment vs Placebo | | |
| Significance vs Placebo | 0.067 | 0.11 |
| 2^{nd} Half of Night | | |
| Number of Subjects | 17 | 8 |
| Number of | 23 | 8 |
| Observations | | |
| Mean Change with | 0.6 | 5.0 |
| Treatment vs Placebo | | |
| Significance vs Placebo | 0.93 | 0.56 |

### Example 3

### Marinol for Apnea: 75% Reduction Analysis

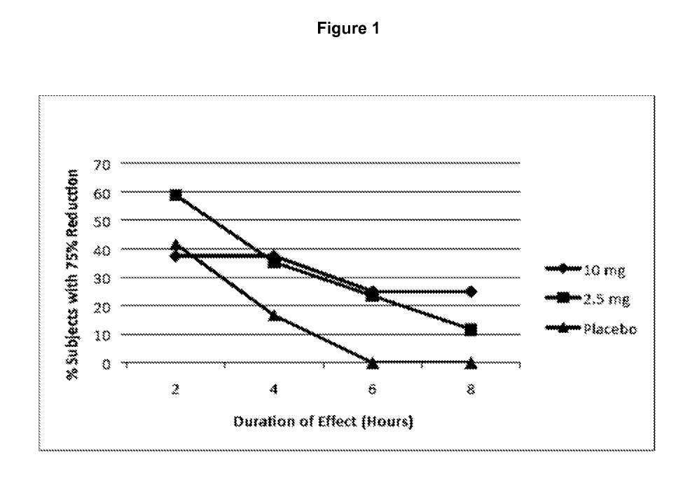

The study from Example 1 was further analyzed with respect to the percentage of subjects demonstrating a 75% reduction in the AHI for 2-, 4-, 6-, and 8-hour consecutive intervals. As shown in FIG. 1, a dose of 2.5 mg (line with square data points) resulted in greater than 60% of the subjects showing a ≧75% reduction (versus baseline) in AHI for at least 2 consecutive hours. In contrast, a dose of 10 mg (line with diamond data points) resulted in fewer than 30% of the subjects showing a 2-hour reduction in AHI of ≧75%. This same phenomenon was seen with respect to a four-hour response interval. Thus, for a 2 and 4 hour treatment window, 2.5 mg of Marinol was more effective in these patients than a 10 mg dose. In contrast to the expected sigmoidal dose-response curve that typifies most drug therapies, THC effect on cannabinoid-sensitive disorders such as apnea is consistent with a non-monotonic response of the inverted U. Thus, a superior medicament of the present invention produces a threshold plasma THC concentration but does not reach the decreasing response portion of the dose curve.

### Example 4

### Marinol for Apnea: Dose and Time Dependence

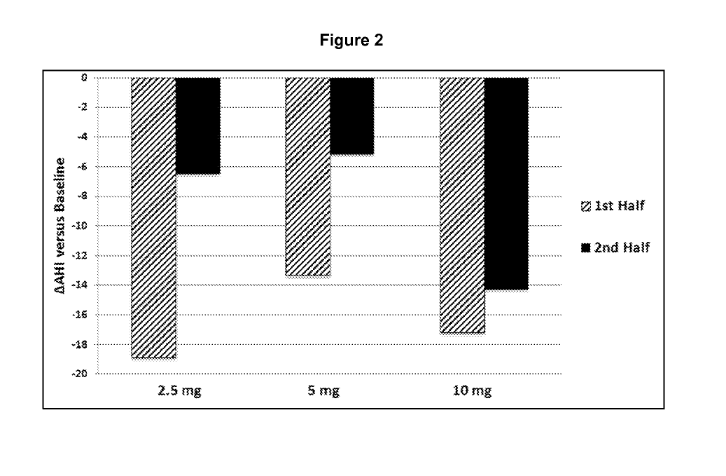

The study from Example 1 was further analyzed for efficacy and dose response of THC with respect to AHI during early (T₀−T₄) and late (T₅−T₈) treatment windows. In the results are shown in FIG. 2; the early treatment window is indicated by stippled bars and the late treatment window is indicated by solid bars.

The orally administered instant release cannabinoid provided remarkable efficacy during the early treatment window. Consistent with Example 3, these results further unexpectedly show that 2.5 mg of Marinol was superior to 10 mg which was superior to 5 mg. In contrast, in the late treatment window, 10 mg of Marinol was superior to 2.5 mg and 5 mg. Furthermore, based on the immediate but transient therapeutic effect of the immediate release doses, additional therapeutic efficacy can be provided by adding a delayed-release component to an immediate release component. Thus, these data indicate that a surprising therapeutic benefit is provided by medicaments of the present invention, for example, a medicament which provides a PK profile of PK Profile 1 or any of PK Profile 2 through PK Profile 19.

### Example 5

### Marinol for Apnea: Establishing the Therapeutic Window

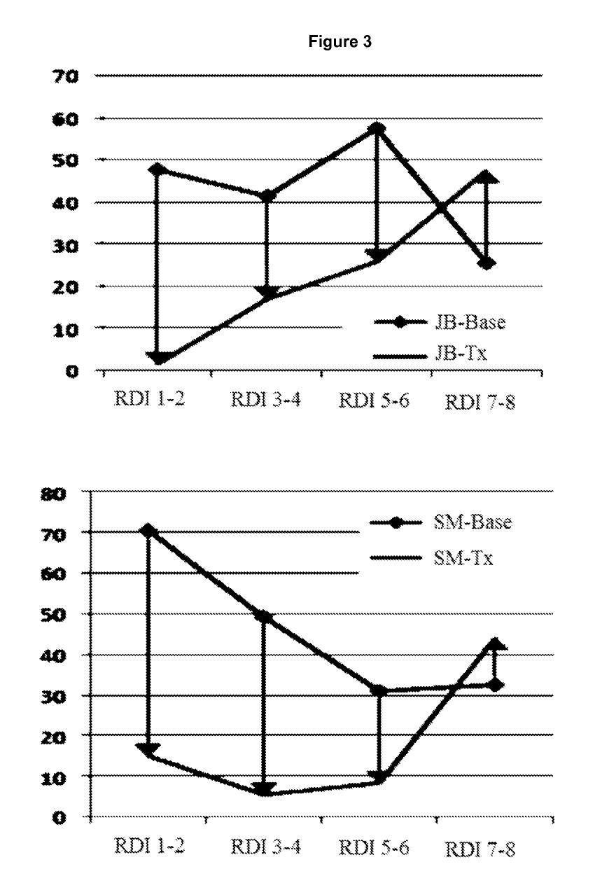

The apnea-hypopnea index (AHI) during a treatment window was calculated for two examplary patients (“JB” and “SM”) for hours T₁−T₂ (RDI1-2), T₃−T₄ (RDI1-2), T₅-T₆ (RDI1-2), and T₇−T₈ (RDI1-2). The patients had each taken a single 2.5 mg dose of Marinol 30 minutes before bed. The results, as shown in FIG. 3, are of the baseline (lines with diamond data points) and the treatment (lines without symbols). A single 2.5 mg immediate release dose of cannabinoid (Marinol) provided a significant therapeutic effect during an early treatment window but provided a reduced therapeutic effect during a late treatment window. These data are consistent with the arousal index data presented in Table 6.

Based on the immediate but transient therapeutic effect of the immediate release dosage, these results support the formulation for a combined immediate and delayed-release medicament.

These results further support the superior therapeutic efficacy of the PK profiles and present medicaments for apnea patients throughout a normal sleeping period.

### Example 6

### Oral Dosing

For the purpose of comparing oral medicaments (i.e., providing a comparator), a medicament is orally administered to one or more subjects. The various factors affecting ADME (absorption, distribution, metabolism, and excretion) of the drug are standardized. For example, the patient is optionally a fasted patient and optionally falls asleep within one of 15 minutes or 30 minutes of laying down for bed. The same subjects are used for comparing different medicaments (after providing an appropriate wash-out period).

Plasma levels of the drug (e.g. THC) and metabolites thereof (e.g. 11-OH-THC) are taken at regular intervals (e.g. every 30 minutes) during a treatment window (e.g. from T₀ to T₈ₕᵣ).

Therapeutic responses are recorded throughout the entire treatment window.

Therapeutic responses are correlated with pharmacokinetic parameters of the drug or metabolites thereof. The pharmacokinetic parameters include one or more of: plasma concentration and AUC (at various times).

### Example 7

### Oral Dosing Comparator 1

An oral dosing “comparator” is provided by orally administering an immediate release dosage of cannabinoid (e.g. MARINOL® soft gel).

The therapeutic response is assessed. AHI is improved in subjects receiving 1 to 75 mg. For subjects receiving lower doses of THC (e.g. 5 mg-20 mg), the AHI revealed a period of therapeutic efficacy following a latency period. The therapeutic window does not consistently extend through the seventh or eighth hour of sleep.

### Example 8

### Oral Dosing Comparator 2

A second oral dosing comparator is provided by the method of Example 6, where a first immediate release dosage of cannabinoid (e.g. 2.5 mg MARINOL® soft gel) to a sleep apnea patient 30 minutes before bed at Tₒ. The patient is allowed to fall asleep and at a predetermined time, the subject is briefly woken to receive a second immediate release dosage of MARINOL® soft gel (e.g. at T₃) and allowed to return to sleep.

The therapeutic response is assessed. AHI is improved in subjects receiving 1 to 75 mg. For subjects receiving lower doses of THC (e.g. 5 mg-20 mg), the AHI reveals a period of therapeutic efficacy following a latency period. The therapeutic window generally extends through the seventh or eighth hour of sleep.

### Example 9

### Predictable Plasma Levels

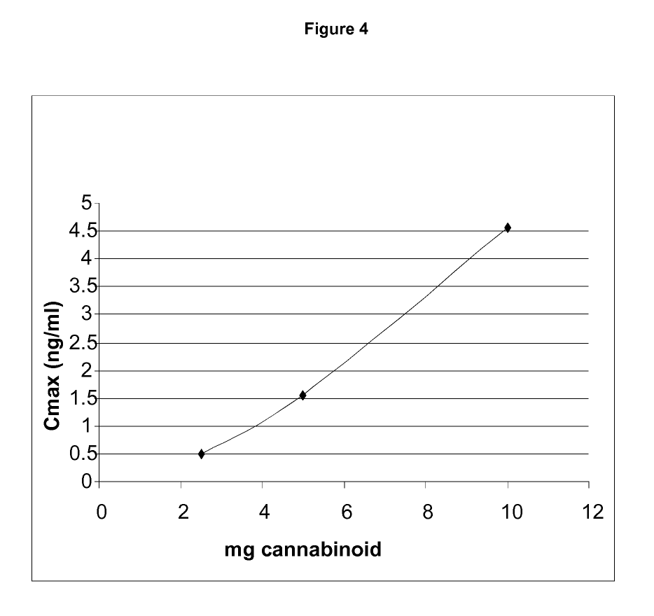

FIG. 4 depicts the relationship of Cmax (ng/ml) and cannabinoid amount (mg) for an immediate release dosage compartment (Marinol formulation). As can be seen from FIG. 4, given the drug dose, plasma levels such as Cmax are predictable from an immediate release dosage compartment.

### Example 10

### In-Vitro Dissolution Assays

The rate of extraction of a drug such as a cannabinoid from a medicament is a key contributor to the resulting PK profile. One method of tailoring a medicament to a desired PK profile involves the use of an in-vitro dissolution assay that mimics or approximates the conditions experienced by the drug in the GI tract. This assay can be used to predict the rate and/or efficiency of drug extraction after administration.

Any dissolution assay is useful when tailoring the PK profile of a medicament. Various assays are known, for example, USP Apparatus 1 (basket), 2 (paddle), 3 (recipricating cylinder), and 4 (flow-through cell). In these assays, a medicament is placed in an appropriate medium (or media) and the medicament is evaluated by sampling the medium (or media) periodically and determining the fraction or percent of drug dissolved or released at each sampling time.

An examplary dissolution method follows. Using USP Apparatus II (paddles) evaluate the medicament for 2 hours in 750 mL of 0.1 N HCl. Operate the rotation speed at 50-75 RPM in medium held at 37 C. At two hours add 125 mL of previously heated 0.2M Sodium Phosphate Tribasic and adjust the pH to 6.0 with 2N HCl or 2N NaOH. Evaluate the medicament in this medium for 2 hours (total time 2 to 4 hours). At 4 hours, add an additional 125 mL of previously heated 0.2M Sodium Phosphate Tribasic and adjust pH to 6.8 to 7.4 with 2N HCl or 2N NaOH. Evaluate the drug from 4 to 6 hours in this final medium. Note that a surfactant or solvent may be necessary in order to obtain appropriate sink conditions.

### Example 11

### Medicament: Immediate Release (IR) plus Delayed Release (DR) Compartments

A medicament is provided comprising a delayed release compartment and an immediate release compartment. By example, the delayed release compartment is an aqueous or semi-aqueous liquid compartment comprising:

a) cannabinoid (e.g. 9−THC)— from about 2 to about 10 mg/ml (e.g. about 5 mg/ml);

b) ethanol—from 15% to about 65% (e.g. about 45%);

c) propylene glycol—from about 0.1 to about 25% (e.g. about 5%);

d) polyethylene glycol—from about 1% to about 25% (e.g. about 10%);

e) buffered (e.g. pH 7) aqueous solution (e.g. phosphate buffer)—from about 10% to about 60% (e.g. about 37%); and

f) a viscosity modifier (e.g. hydroxypropyl cellulose) in a viscosity modifying amount (e.g. about 0.3%).

Based on the profile data shown in FIG. 5 for such a delayed release compartment (reproduced from FIG. 6 of US 2009/0181080), a medicament of the present invention is produced by combining the delayed release compartment with one or more additional compartments (e.g. an immediate release compartment) to provide a PK profile of at least one of PK Profile 1 through PK Profile 19.

With the teaching here, the skilled artisan can now modify this type of formulation to meet the target PK profile. For example, the following modification approaches are useful:
Adjust drug to release modifier ratio
Adjust concentration of viscosity modifier (typically increasing the concentration slows release)
Adjust the grade of the viscosity modifier (using a higher molecular weight polymer typically slows release)
Use different viscosity modifiers (separate material or combinations of materials)

### Example 12

### Medicament: Plurality of Pellets, Continuous Release

A medicament is provided comprising a delayed release compartment and an immediate release compartment contained within the same dose unit. By example, the immediate release compartment is a plurality of solid pellets (or microspheres) and the delayed release compartment is a plurality of solid pellets comprising a delayed release modifier coating.

TABLE 7
Pellet in Capsule (IR + DR)
10 mg Dronabinol (or other THC) Capsule

| | | | |
|---|---|---|---|
| No. | Ingredient | mg/capsule | % w/w |

Immediate Release Pellets/Compartment

| | | | |
|---|---|---|---|
| 1 | Dronabinol | 2.5 | 1.16 |
| 2 | Sodium Lauryl Sulfate | 2.5 | 1.16 |
| 3 | Neusilin US2 (Magnesium | 20.0 | 9.30 |
| | Aluminometasilicate) | | |
| 4 | Avicel PH101 (Microcrystalline | 25.0 | 11.63 |
| | Cellulose) | | |
| | SubTotal | 50.0 | 23.3 |

Delayed Release Pellets/Compartment

| | | | |
|---|---|---|---|
| 5 | Dronabinol | 7.5 | 3.49 |
| 6 | Sodium Lauryl Sulfate | 7.5 | 3.49 |
| 7 | Neusilin US2 (Magnesium | 60.0 | 27.91 |
| | Aluminometasilicate) | | |
| 8 | Avicel PH101 (Microcrystalline | 75.0 | 34.88 |
| | Cellulose) | | |
| 9 | Ethylcellulose (Aquacoat ECD 30% | 11.4 | 5.30 |
| | w/w dispersion) | | |
| 10 | Dibutyl Sebecate | 3.6 | 1.67 |
| | SubTotal | 165.0 | 76.7 |
| | Total Capsule Fill Weight | 215.0 | 100.0 |

Manufacturing Process for Dronabinol 10% w/w Pellets. These pellets represent an immediate release compartment when uncoated and a delayed release compartment when coated. Mixtures of the two in a capsule shell provide for a combination of immediate- and delayed-release compartments. The ratio of the two pellets types allows for variation in weighting of the dose fraction between the two compartments. This example uses a 2.5 mg dose in the immediate release compartment and a 7.5 mg dose in the delayed release compartment.
A) Dissolve Dronabinol (1+5) in Ethanol (200 proof) in a suitable tank and mixer.
B) Disperse Sodium Lauryl Sulfate (2+6) into (A). Continue to mix.
C) Charge Neusilin (3+7) to a high shear granulator.
D) While mixing the Neusilin, add the dispersion from (B). Mix until (B) is suitably dispersed.
E) Transfer the wet contents of the high shear granulator to a tray dryer.
F) Dry at 50 C (+/−10 C) to remove the Ethanol.
G) Collect the dried material from the trays and pass through a No. 20 mesh screen to deagglomerate.
H) Charge the Dronabinol-Loaded Neusilin (G) to a high shear granulator.
I) Charge the required quantity of Avicel PH101 (4+8) to the high shear granulator.
J) Mix the contents in the high shear granulator for 5 minutes.
K) While mixing, add sufficient purified water to wet mass.
L) Transfer the wet mass (K) to a suitable cold mass extruder fitted with a dome screen of suitable size (e.g. 0.8 mm-1.2 mm).
M) Extrude the wet mass to collect strands.
N) Load the collected strands (M) into a marumerizer. Operate the marumerizer to produce pellets.
O) Extrude/marumerize the entire wet mass (K) from the high shear granulator.
P) Transfer the wet pellets (O) to a fluid bed or tray dryer and dry.
Q) Collect the dried pellets (P) from the dryer into a suitable container.
R) Using screens, collect pellets that pass through a No. 18 mesh screen but are retained on a No. 30 mesh screen.

Dronabinol DR Pellets
S) Prepare the required quantity of Ethylcellulose Coating Dispersion by adding the Dibutyl Sebecate to Aquacoat in a suitable tank with mixer.
T) Stir the Aquacoat/Dibutyl Sebecate dispersion for not less than 30 minutes.
U) Add sufficient purified water to dilute (T) to a 20% w/w dispersion. Mix the dispersion continuously.
V) Charge the required quantity of Dronabinol 10% w/w Pellets (18-30 mesh) to a fluid bed coater with bottom spray and a Wurster column insert.
W) Apply a 10% weight gain of Ethylcellulose Coating Dispersion (U) to the fluidized pellets.
X) Allow the pellets to dry in the fluid bed coater for 15 to 30 minutes after the completion of coating.
Y) Collect the Dronabinol DR Pellets from the fluid bed coater.

Encapsulation

Z) Using a suitable encapsulator, fill suitably sized two piece hard capsule shells with 50 mg of Dronabinol IR 10% w/w Pellets (18-30 mesh) and 165 mg of Dronabinol DR Pellets.

Modifications.

With the teaching here, the skilled artisan can now modify this type of formulation to meet the target PK profile. For example, the following modification approaches are useful:
Adjust drug to surfactant ratio
Use different surfactants
Adjust weight gain of ethylcellulose coating (typically higher weight gains decrease drug release)
Use a film modifier (e.g. the addition of hypromellose in an ethylcellulose film increases diffusion or can change the rate of release by making it more hydrophilic)
Adjust the quantity or types of film additives such as the plasticizer
Use a different film forming polymer
Use combinations of different polymers (can add enteric polymers to ethylcellulose to increase diffusion as the pH of the GI tract increases)
Use post coating drying/curing to affect film properties

### Example 13

### Bilayer/Matrix Tablet

A medicament is provided comprising a delayed release compartment and an immediate release compartment contained with the same dose unit. By example, the immediate release compartment is a distinct solid matrix layer without release modifying excipients and the delayed release compartment is a solid matrix layer comprising release modifying agents. The medicament provided in this Example is a solid, adsorbate to facilitate solids handling of dronabinol and incorporation into the solid phase immediate- and delayed-release compartments. The delayed-release compartment releases portions of the dronabinol dose over time in a continuous manner.

TABLE 8
Bilayer/Matrix Tablet
10 mg Dronabinol (or other THC) Tablet

| | | | |
|---|---|---|---|
| No. | Ingredient | mg/tablet | % w/w |

Immediate Release Layer/Compartment

| | | | |
|---|---|---|---|
| 1 | Dronabinol | 2.5 | 0.61 |
| 2 | Sodium Lauryl Sulfate | 2.5 | 0.61 |
| 3 | Fujicalin (Dicalcium Phosphate Anhydrous) | 50.0 | 12.14 |
| 4 | Prosolv SMCC 90HD (Silicified | 39.5 | 9.59 |
| | Microcrystalline Cellulose) | | |
| 5 | Croscarmellose Sodium | 5.0 | 1.21 |
| 6 | Magnesium Stearate | 0.5 | 0.12 |
| | SubTotal | 100.0 | 24.27 |

Delayed Release Layer/Compartment

| | | | |
|---|---|---|---|
| 7 | Dronabinol | 7.5 | 1.82 |
| 8 | Sodium Lauryl Sulfate | 7.5 | 1.82 |
| 9 | Fujicalin (Dicalcium Phosphate Anhydrous) | 150.0 | 36.41 |
| 10 | Prosolv SMCC 90HD (Silicified | 42.0 | 10.19 |
| | Microcrystalline Cellulose) | | |
| 11 | Methocel K4M Premium DC (Hypromellose | 90.0 | 21.84 |
| | Type 2208) | | |
| 12 | Colloidal Silicon Dioxide | 1.5 | 0.36 |
| 13 | Magnesium Stearate | 1.5 | 0.36 |
| | SubTotal | 300.0 | 72.82 |

Outer Trade Dress Coating (Non release modifying)

| | | | |
|---|---|---|---|
| 14 | Opadry Colored Coating System - PVA | 12.0 | 2.91 |
| | Based | | |
| | Total Tablet Weight | 412.0 | 100.00 |

Manufacturing Process for Dronabinol Loading onto Fujicalin (Adsorbate Formation)
A) Dissolve Dronabinol (1+7) in Ethanol (200 proof) in a suitable tank with a mixer.
B) Disperse Sodium Lauryl Sulfate (2+8) into (A). Continue to mix.
C) Charge Fujicalin (3+9) to a high shear granulator.
D) While mixing the Fujicalin, add the dispersion from (B). Mix until (B) is suitably dispersed.
E) Transfer the wet contents of the high shear granulator to a tray dryer.
F) Dry at 50 C (+/−10 C) to remove the Ethanol.
G) Collect the dried material from the trays and pass through a No. 20 mesh screen to deagglomerate.
H) Store in an HPDE drum double lined with PE bags and desiccants as required.

Immediate Release Layer Blend/Compartment. The prepared powder blend provides immediate release of the dronabinol from the compressed tablet.
I) Charge the required quantity of the Dronabinol-Loaded Fujicalin (H) into a tumble blender after passing through a No. 20 mesh screen.
J) Charge the required quantity of Prosolv (4) to the tumble blender after passing through a No. 20 mesh screen.
K) Close the blender and blend for 15 minutes.
L) Open the blender and charge the required quantity of Croscarmellose Sodium (5) after passing through a No. 20 mesh screen.
M) Close the blender and blend for 5 minutes.
N) Open the blender and charge the required quantity of Magnesium Stearate (6) after passing through a No. 20 mesh screen.
O) Close the blender and blend for 2 minutes.
P) Collect the IR Layer Blend (O) from the blender into an HDPE drum double lined with PE bags and desiccants as required.

Delayed Release Layer Blend/Compartment. The prepared powder blend provides for a delayed release of dronabinol from the compressed tablet using Methocel as a rate modifying excipient.
Q) Charge the required quantity of the Dronabinol-Loaded Fujicalin (H) into a tumble blender after passing through a No. 20 mesh screen.
R) Charge the required quantity of Prosolv (10) and Methocel (11) to the tumble blender after passing through a No. 20 mesh screen.
S) Close the blender and blend for 15 minutes.
T) Open the blender and charge the required quantity of Colloidal Silicon Dioxide (12) after passing through a No. 20 mesh screen.
U) Close the blender and blend for 5 minutes.
V) Open the blender and charge the required quantity of Magnesium Stearate (13) after passing through a No. 20 mesh screen.
W) Close the blender and blend for 2 minutes.
X) Collect the DR Layer Blend (W) from the blender into an HDPE drum double lined with PE bags and desiccants as required.

Tablet Compression. As instructed below, the immediate release compartment comprises 25% of the total dose (e.g. 2.5 mg), and the delayed release compartment comprises 75% of the total dose (e.g. 7.5 mg). The combination of the two layers provides for both distinct IR and DR compartments in a tablet.
Y) Using a dual/bi-layer tablet press, compress tablets containing 100 mg of the IR Layer Blend/Compartment and 300 mg of the DR Layer Blend/Compartment.
Z) Collect the Dronabinol 10 mg Tablets into HDPE drums lined with PE bags and desiccants as required.

Tablet Coating (Non-release modifying trade dress)
AA) Prepare the Opadry Coating System as an 18% w/w dispersion in water using a suitable tank and mixer.
AB) Load the Dronabinol 10 mg Tablets (Z) into a suitable tablet coater.
AC) Apply a 3% weight gain of the Opadry Coating dispersion to the tablets.
AD) Collect the coated tablets from the tablet coater into HDPE drums lined with PE bags and desiccants as required.

Modifications.

With the teaching here, the skilled artisan can now modify this type of formulation to meet the target PK profile. For example, the following modification approaches are useful:
Adjust the ratio of the rate controlling matrix excipient (typically increasing concentration slows release)
Adjust the grade or type of matrix excipient (typically increasing molecular weight of the polymer slows release)
Use different types of rate controlling matrix excipients (polymers, waxes, etc.)
Adjust the geometry of the tablet (tablet shapes the minimize their surface area to volume ratio slow drug release)
Adjust tablet properties (adjusting the porosity of the tablet by making them harder (less porous) or softer (more porous) affects release rate)
Use post compression curing to affect release (e.g. can heat tablets prepared with a wax to modify release)
Can apply a release modifying coating over the entire dosage form to add additional options

### Example 14

### Medicament: 2 Pulse, Enteric Coating

A medicament is provided comprising a delayed release compartment and an immediate release compartment contained within the same dose unit. By example, the immediate release compartment is a plurality of solid pellets (or microspheres) and the delayed release compartment is a plurality of solid pellets comprising a delayed release modifier (pH dependent, enteric coating). The medicament provided in this example is a solid, adsorbate to facilitate solids handling of dronabinol and incorporation into a solid phase immediate- and delayed-release compartment. The delayed-release compartment releases the entire dose of the dronabinol when the pH of the gastrointestinal tract is sufficiently alkaline (e.g. pH>5.5) to dissolve the coating. Since the coating is insoluble in acidic conditions, the delayed release compartment is released in the small intestine unlike the immediate release compartment which releases in the stomach.

TABLE 9
Pellet in Capsule - (IR + DR)
10 mg Dronabinol (or other THC) Capsule

| | | | |
|---|---|---|---|
| No. | Ingredient | mg/capsule | % w/w |

Immediate Release Pellets/Compartment

| | | | |
|---|---|---|---|
| 1 | Dronabinol | 2.5 | 1.12 |
| 2 | Sodium Lauryl Sulfate | 2.5 | 1.12 |
| 3 | Neusilin US2 (Magnesium | 20.0 | 8.99 |
| | Aluminometasilicate) | | |
| 4 | Avicel PH101 (Microcrystalline Cellulose) | 25.0 | 11.24 |
| | SubTotal | 50.0 | 22.47 |

Delayed Release Enteric Coated Pellets/Compartment

| | | | |
|---|---|---|---|
| 5 | Dronabinol | 7.5 | 3.37 |
| 6 | Sodium Lauryl Sulfate | 7.5 | 3.37 |
| 7 | Neusilin US2 (Magnesium | 60.0 | 26.97 |
| | Aluminometasilicate) | | |
| 8 | Avicel PH101 (Microcrystalline Cellulose) | 75.0 | 33.71 |
| 9 | Methacrylic Acid Copolymer, Type C | 13.2 | 5.93 |
| | (Eudragit L30D-55 30% w/w dispersion) | | |
| 10 | Triethyl Citrate | 2.7 | 1.21 |
| 11 | Talc | 6.6 | 2.97 |
| | SubTotal | 172.5 | 77.53 |
| | Total Capsule Fill Weight | 222.5 | 100.0 |

Manufacturing Process for Dronabinol 10% w/w Pellets. These pellets represent an immediate release compartment when uncoated and a delayed release compartment when coated. Mixtures of the two in a capsule shell provide for a combination of immediate- and delayed-release compartments. The ratio of the two pellets types allows for variation in weighting of the dose fraction between the two compartments. This example uses a 2.5 mg dose in the immediate release compartment and a 7.5 mg dose in the delayed release compartment.
A) Dissolve Dronabinol (1+5) in Ethanol (200 proof) in a suitable tank and mixer.
B) Disperse Sodium Lauryl Sulfate (2+6) into (A). Continue to mix.
C) Charge Neusilin (3+7) to a high shear granulator.
D) While mixing the Neusilin, add the dispersion from (B). Mix until (B) is suitably dispersed.
E) Transfer the wet contents of the high shear granulator to a tray dryer.
F) Dry at 50 C (+/−10 C) to remove the Ethanol.
G) Collect the dried material from the trays and pass through a No. 20 mesh screen to deagglomerate.
H) Charge the Dronabinol-Loaded Neusilin (G) to a high shear granulator.
I) Charge the required quantity of Avicel PH101 (4+8) to the high shear granulator.
J) Mix the contents in the high shear granulator for 5 minutes.
K) While mixing, add sufficient purified water to wet mass.
L) Transfer the wet mass (K) to a suitable cold mass extruder fitted with a dome screen of suitable size (e.g. 0.8 mm-1.2 mm).
M) Extrude the wet mass to collect strands.
N) Load the collected strands (M) into a marumerizer. Operate the marumerizer to produce pellets.
O) Extrude/marumerize the entire wet mass (K) from the high shear granulator.
P) Transfer the wet pellets (O) to a fluid bed or tray dryer and dry.
Q) Collect the dried pellets (P) from the fluid bed dryer into a suitable container.
R) Using screens, collect pellets that pass through a No. 18 mesh screen but are retained on a No. 30 mesh screen.

Dronabinol DR Pellets
S) Prepare the required quantity of Methacrylic Acid Copolymer Coating Dispersion by adding the Triethyl Citrate to Eudragit L30D-55 in a suitable tank with mixer.
T) Stir the Eudragit/Triethyl Citrate dispersion for not less than 30 minutes.
U) Disperse the required quantity of talc in sufficient purified water to provide a final solids content of the coating suspension at 15% w/w.
V) Combine the Eudragit/Triethyl Citrate dispersion and the talc/water dispersion. Mix the dispersion continuously.
W) Charge the required quantity of Dronabinol 10% w/w Pellets (18-30 mesh) to a fluid bed coater with bottom spray and a Wurster column insert.
X) Apply a 15% weight gain of Methacrylic Acid Coating Dispersion (W) to the fluidized pellets.
Y) Allow the pellets to dry in the fluid bed coater for 15 to 30 minutes after the completion of coating.
Z) Collect the Dronabinol DR Pellets from the fluid bed coater.

Encapsulation
AA) Using a suitable encapsulator, fill suitably sized two piece hard capsule shells with 50 mg of Dronabinol IR 10% w/w Pellets (18-30 mesh) and 172.5 mg of Dronabinol DR Pellets.

Modifications.

With the teaching here, the skilled artisan can now modify this type of formulation to meet the target PK profile. For example, the following modification approaches are useful:
For enteric coatings, increasing coating weight gain can be used to blunt release
Use different polymers or polymer grades to adjust trigger points (i.e. Eudragit L30D-55/L100-55 is meant to release at pH>5.5, Eudragit L100 at pH>6, Eudragit FS30D at pH>7.2, Eudragit S100 at pH>7.4 while HPMCAS-L at pH>5.5, HPMCAS-M at pH>6.0, and HPMCAS-H at pH>6.8). Eudragit is based on methacrylic acid while HPMCAS is based upon hypromellose acetate succinate. Other enterics include cellulose acetate phthalate, polyvinyl acetate phthalate, shellac, and others.
Use release modifiers in enteric films to modify release further
Use alternative film additives (e.g. alternative plasticizer and glidants)

### Example 15

### Medicament: 3 Pulse, Enteric Coating

A medicament is provided comprising 2 delayed release compartments and an immediate release compartment within the same dose unit. By example, the immediate release compartment contains solid pellets (or microspheres) and the delayed release compartments each have solid pellets comprising different delayed release modifier (pH dependent, enteric) coatings. The medicament provided in this example is a solid, adsorbate to facilitate solids handling of dronabinol and incorporation into a solid phase immediate- and delayed-release compartment.

Upon administration, the IR compartment releases the dronabinol and the DR. compartment releases its dose of dronabinol when the pH of the gastrointestinal tract is sufficiently alkaline (e.g. pH>5.5 and pH>7.2) to dissolve the different coatings (e.g. DR1 and DR2, respectively). Since the coatings are insoluble in acidic conditions, the delayed release compartments release in the upper portion of the small intestine and in the lower portion of the small intestine/large intestine, respectively, unlike the immediate release compartment which releases in the stomach.

TABLE 10
Pellet in Capsule - (IR + DR1 + DR2)
10 mg Dronabinol (or other THC) Capsule

| | | | |
|---|---|---|---|
| No. | Ingredient | mg/capsule | % w/w |

Immediate Release Pellets/Compartment

| | | | |
|---|---|---|---|
| 1 | Dronabinol | 2.5 | 1.12 |
| 2 | Sodium Lauryl Sulfate | 2.5 | 1.12 |
| 3 | Neusilin US2 (Magnesium | 20.0 | 8.99 |
| | Aluminometasilicate) | | |
| 4 | Avicel PH101 (Microcrystalline Cellulose) | 25.0 | 11.24 |
| | SubTotal | 50.0 | 22.5 |

Delayed Release Enteric Coated Pellets/Compartment (DR1)

| | | | |
|---|---|---|---|
| 5 | Dronabinol | 5.0 | 2.25 |
| 6 | Sodium Lauryl Sulfate | 5.0 | 2.25 |
| 7 | Neusilin US2 (Magnesium | 40.0 | 17.98 |
| | Aluminometasilicate) | | |
| 8 | Avicel PH101 (Microcrystalline Cellulose) | 50.0 | 22.47 |
| 9 | Methacrylic Acid Copolymer, Type C | 8.8 | 3.96 |
| | (Eudragit L30D-55 30% w/w dispersion) | | |
| 10 | Triethyl Citrate | 1.8 | 0.81 |
| 11 | Talc | 4.4 | 1.98 |
| | SubTotal | 115.0 | 51.7 |

Delayed Release Enteric Coated Pellets/Compartment (DR2)

| | | | |
|---|---|---|---|
| 12 | Dronabinol | 2.5 | 1.12 |
| 13 | Sodium Lauryl Sulfate | 2.5 | 1.12 |
| 14 | Neusilin US2 (Magnesium | 20.0 | 8.99 |
| | Aluminometasilicate) | | |
| 15 | Avicel PH101 (Microcrystalline Cellulose) | 25.0 | 11.24 |
| 16 | Eudragit FS30D (30% w/w dispersion) | 7.0 | 3.15 |
| 17 | Triethyl Citrate | 0.2 | 0.09 |
| 18 | Glycerol Monostearate | 0.3 | 0.13 |
| | SubTotal | 57.5 | 25.8 |
| | Total Capsule Fill Weight | 222.5 | 100.0 |

Manufacturing Process for Dronabinol 10% w/w Pellets. These pellets represent an immediate release compartment when uncoated and delayed release compartments when coated with different enteric polymers. Mixtures of the three in a capsule shell provide for a combination of immediate-(IR) and delayed-release compartments (DR1 and DR2). The ratio of the three pellets types allows for variation in weighting of the dose fraction between the two main compartments (e.g. IR and DR) with further refinement in the DR compartment (e.g. DR1 and DR2). This example uses a 2.5 mg dose in the immediate release compartment and a 7.5 mg dose in the delayed release compartment (5.0 mg dose in DR1 and 2.5 mg dose in DR2).
A) Dissolve Dronabinol (1+5+12) in Ethanol (200 proof) in a suitable tank and mixer.
B) Disperse Sodium Lauryl Sulfate (2+6+13) into (A). Continue to mix.
C) Charge Neusilin (3+7+14) to a high shear granulator.
D) While mixing the Neusilin, add the dispersion from (B). Mix until (B) is suitably dispersed.
E) Transfer the wet contents of the high shear granulator to a tray dryer.
F) Dry at 50 C (+/−10 C) to remove the Ethanol.
G) Collect the dried material from the trays and pass through a No. 20 mesh screen to deagglomerate.
H) Charge the Dronabinol-Loaded Neusilin (G) to a high shear granulator.
I) Charge the required quantity of Avicel PH101 (4+8-F15) to the high shear granulator.
J) Mix the contents in the high shear granulator for 5 minutes.
K) While mixing, add sufficient purified water to wet mass.
L) Transfer the wet mass (K) to a suitable cold mass extruder fitted with a dome screen of suitable size (e.g. 0.8 mm-1.2 mm).
M) Extrude the wet mass to collect strands.
N) Load the collected strands (M) into a marumerizer. Operate the marumerizer to produce pellets.
O) Extrude/marumerize the entire wet mass (K) from the high shear granulator.
P) Transfer the wet pellets (O) to a fluid bed or tray dryer and dry.
Q) Collect the dried pellets (P) from the fluid bed dryer into a suitable container.
R) Using screens, collect pellets that pass through a No. 18 mesh screen but are retained on a No. 30 mesh screen.

Dronabinol DR1Pellets
S) Prepare the required quantity of Methacrylic Acid Copolymer Coating Dispersion by adding the Triethyl Citrate to Eudragit L30D-55 in a suitable tank with mixer.
T) Stir the Eudragit/Triethyl Citrate dispersion for not less than 30 minutes.
U) Disperse the required quantity of talc in sufficient purified water to provide a final solids content of the coating suspension at 15% w/w.
V) Combine the Eudragit/Triethyl Citrate dispersion and the talc/water dispersion. Mix the dispersion continuously.
W) Charge the required quantity of Dronabinol 10% w/w Pellets (18-30 mesh) to a fluid bed coater with bottom spray and a Wurster column insert.
X) Apply a 15% weight gain of Methacrylic Acid Coating Dispersion (W) to the fluidized pellets.
Y) Allow the pellets to dry in the fluid bed coater for 15 to 30 minutes after the completion of coating.
Z) Collect the Dronabinol DR1Pellets from the fluid bed coater.

Dronabinol DR2Pellets
AA) Prepare the required quantity of Eudragit FS30D Coating Dispersion.
BB) Combine the required quantity of purified water (sufficient to provide a final solids content of 20% w/w), triethyl citrate, and glycerol monostearate.
CC) Heat the water/triethyl citrate/glycerol monostearate dispersion to 50 C-60 C. Use a high shear homogenizer to mix.
DD) Allow dispersion (CC) to cool to <30 C with mixing then combine with required quantity of Eudragit FS30D. Mix throughout coating.
EE) Charge the required quantity of Dronabinol 10% w/w Pellets (18-30 mesh) to a fluid bed coater with bottom spray and a Wurster column insert.
FF) Apply a 15% weight gain of Eudragit FS30D Coating Dispersion (EE) to the fluidized pellets.
GG) Allow the pellets to dry in the fluid bed coater for 15 to 30 minutes after the completion of coating.
HH) Collect the Dronabinol DR2Pellets from the fluid bed coater.

Encapsulation
II) Using a suitable encapsulator, fill suitably sized two piece hard capsule shells with 50 mg of Dronabinol IR 10% w/w Pellets (18-30 mesh), 115 mg of Dronabinol DR1Pellets, and 57.5 mg of Dronabinol DR2Pellets.

Modifications.

With the teaching here, the skilled artisan can now modify this type of formulation to meet the target PK profile. For example, the following modification approaches are useful:
The modifications set forth in Example 14
Combine an IR pellet with an enteric coated pellet and a delayed release pellet like in Example 12

### Example 16

### Medicament: Two Pulse Enteric Coating

A medicament is provided comprising a delayed release compartment and a separate immediate release compartment contained within the same dose unit or package. By example, the immediate release compartment is liquid compartment and the delayed release compartment is a liquid compartment comprising a delayed release modifier (pH dependent, enteric) coating. The delayed-release compartment in this example is prepared by coating a liquid compartment with an enteric polymer. The delayed-release compartment releases the entire dose of the dronabinol when the pH of the gastrointestinal tract is sufficiently alkaline (e.g. pH>5.5) to dissolve the coating. Since the coating is insoluble in acidic conditions, the delayed release compartment is released in the small intestine unlike the immediate release liquid compartment which releases in the stomach.

TABLE 11
Two Pulse/Coated Soft Gels
10 mg Dronabinol Capsules

| | | |
|---|---|---|
| | | g/100 g Coating |
| No. | Ingredient | Suspension |

Immediate Release Capsule Unit/Compartment

| | |
|---|---|
| 1 | Marinol Capsule 5 mg (NDC 0051-0022-21) |

Enteric Coated Capsule Unit/Delayed-Release Compartment
Substrate

| | | |
|---|---|---|
| 2 | Marinol Capsule 5 mg (NDC 0051-0022-21) | |
| | Enteric Coating | |
| 3 | Methacrylic Acid Copolymer, Type C | 29.4 |
| | (Eudragit L30D-55 30% w/w dispersion) | |
| 4 | Triethyl Citrate | 1.8 |
| 5 | Talc | 4.4 |
| 6 | Purified Water | 64.4 |

Preparation of Coating Suspension for the DR Component
A) Combine Eudragit L30D-55 (3) and Triethyl Citrate (4). Stir for at least 30 minutes.
B) In a separate container, disperse the Talc (5) into sufficient Purified Water (6) to provide a final solids content of 15% in the coating dispersion with a high shear homogenizer.
C) Pour the Purified Water/Talc Dispersion into the Eudragit/Triethyl Citrate Dispersion with stirring.
D) Make sufficient coating suspension for the target batch size.

Coating of Substrate
E) Charge the required quantity of Marinol Capsule 5 mg (2) into a perforated pan coating machine.
F) Apply the Coating Suspension to a target weight gain of 10%.

Preparation of Final Medicament
G) In a suitably sized two piece hard capsule, shell combine one immediate release capsule unit (1) and one DR enteric coated capsule unit (F).
H) Close the capsule shell.

Modifications.

With the teaching here, the skilled artisan can now modify this type of formulation to meet the target PK profile. For example, the following modification approaches are useful:
Varying the properties of the starting substrate as set forth in Example 11.
Adjust coatings as set forth in Example 12 and Example 14.
Combine with other DR components as set forth in Example 15.

### Example 17

### Medicament: IR/DR (Monolithic/Matrix Tablet, Continuous)

Here, a medicament is provided comprising a delayed release compartment and an immediate release compartment contained within the same dose unit. This example also illustrates a contiguous release dosage form of the present invention.

The release modification here, in part, is due to spatial localization of the cannabinoid within the dosage form. Thus, the IR compartment is localized to the perimeter (and immediate sub-perimeter or periphery) of the dosage form and the DR compartment is localized within or interior to the dosage form. The drug in the IR compartment is not substantially buried within the release modifying matrix The total dose contained with the medicament is continuously released. This is another example of a solid adsorbate type dosage form of the present invention.

TABLE 12
Monolithic/Matrix Tablet (IR/DR)
10 mg Dronabinol (or other THC) Tablet

| | | | |
|---|---|---|---|
| No. | Ingredient | mg/tablet | % w/w |

Core Matrix Tablet (IR/DR Compartment)

| | | | |
|---|---|---|---|
| 1 | Dronabinol | 10.0 | 2.43 |
| 2 | Sodium Lauryl Sulfate | 10.0 | 2.43 |
| 3 | Fujicalin (Dicalcium Phosphate Anhydrous) | 200.0 | 48.54 |
| 4 | Prosolv SMCC 90HD (Silicified | 98.0 | 23.79 |
| | Microcrystalline Cellulose) | | |
| 5 | Methocel K4M Premium DC (Hypromellose | 80.0 | 19.42 |
| | Type 2208) | | |
| 6 | Magnesium Stearate | 2.0 | 0.49 |
| | SubTotal | 400.0 | 97.1 |

Outer Trade Dress Coating (Non release modifying)

| | | | |
|---|---|---|---|
| 7 | Opadry Colored Coating System - PVA | 12.0 | 2.91 |
| | Based | | |
| | Total Tablet Weight | 412.0 | 100.0 |

Manufacturing Process for Dronabinol Loading onto Fujicalin (Adsorbate Formation)
A) Dissolve Dronabinol (1) in Ethanol (200 proof) in a suitable tank with a mixer.
B) Disperse Sodium Lauryl Sulfate (2) into (A). Continue to mix.
C) Charge Fujicalin (3) to a high shear granulator.
D) While mixing the Fujicalin, add the dispersion from (B). Mix until (B) is suitably dispersed.
E) Transfer the wet contents of the high shear granulator to a tray dryer.
F) Dry at 50 C (+/−10 C) to remove the Ethanol.
G) Collect the dried material from the trays and pass through a No. 20 mesh screen to deagglomerate.
H) Store in an HPDE drum double lined with PE bags and desiccants as required.

Core Matrix Tablets (IR/DR Compartment)
I) Charge the required quantity of the Dronabinol-Loaded Fujicalin (H) into a tumble blender after passing through a No. 20 mesh screen.
J) Charge the required quantity of Prosolv (4) and Methocel (5) to the tumble blender after passing through a No. 20 mesh screen.
K) Close the blender and blend for 15 minutes.
L) Open the blender and charge the required quantity of Magnesium Stearate (6) after passing through a No. 20 mesh screen.
M) Close the blender and blend for 2 minutes.
N) Collect the Powder Blend (M) from the blender into an HDPE drum double lined with PE bags and desiccants as required.

Tablet Compression
O) Using a tablet press, compress tablet matrix cores at a target tablet weight of 400 mg.
P) Collect the Dronabinol 10 mg Tablets into HDPE drums lined with PE bags and desiccants as required.

Tablet Coating (Non-Release Modifying Trade Dress)
Q) Prepare the Opadry Coating System as an 18% w/w dispersion in water using a suitable tank and mixer.
R) Load the Dronabinol 10 mg Tablets (P) into a suitable tablet coater.
S) Apply a 3% weight gain of the Opadry Coating dispersion to the tablets.
T) Collect the coated tablets from the tablet coater into HDPE drums lined with PE bags and desiccants as required.

Modifications.

With the teaching here, the skilled artisan can now modify this type of formulation to meet the target PK profile. For example, the following modification approaches are useful:
Adjust ratio of drug to excipients
Adjust ratio of rate controlling excipients to non-rate controlling excipients
Adjust properties of rate controlling excipients (e.g. molecular weight for polymers)
Combine rate controlling excipients (e.g. Hypromellose Type 2208 15,000 cps grade with 100,000 cps grade, etc.)
Change rate controlling excipients (e.g. different polymers, different type of material such as a wax)
Manipulate porosity of tablet (e.g. make it harder or softer)
Manipulate tablet geometry
Apply an outer release modifying coating
Make adsorbate using a material that further manipulates release of drug substance

### Example 18

### Medicament: IR/DR (Monolithic, Coated Tablet, Continuous)

A medicament is provided comprising a delayed release compartment and an immediate release compartment contained within the same dose unit. The IR and DR compartments are spatially oriented within a “continuous release” tablet as was also illustrated in Example 17.

This medicament is another example of a solid, adsorbate-type and of a coated DR compartment type dosage form.

TABLE 13
Monolithic/Coated Tablet
10 mg Dronabinol (or other THC) Tablet

| | | | |
|---|---|---|---|
| No. | Ingredient | mg/tablet | %w/w |

Core Tablet

| | | | |
|---|---|---|---|
| 1 | Dronabinol | 10.0 | 2.49 |
| 2 | Sodium Lauryl Sulfate | 10.0 | 2.49 |
| 3 | Fujicalin (Dicalcium Phosphate Anhydrous) | 200.0 | 49.85 |
| 4 | Prosolv SMCC 90HD (Silicified | 98.3 | 24.50 |
| | Microcrystalline Cellulose) | | |
| 5 | Hydropropyl Cellulose (Klucel LF) | 20.0 | 4.99 |
| 6 | Magnesium Stearate | 1.7 | 0.42 |

Controlled Release Coating (IR/DR Compartment)

| | | | |
|---|---|---|---|
| 7 | Ammonio Methacrylate Copolymer, Type A | 7.5 | 1.87 |
| | (Eudragit RL30D 30% Dispersion) | | |
| 8 | Ammonio Methacrylate Copolymer, Type B | 22.5 | 5.61 |
| | (Eudragit RS30D 30% Dispersion) | | |
| 9 | Triethyl Citrate | 6.0 | 1.50 |
| 10 | Talc | 15.0 | 3.74 |

Outer Trade Dress Coating (Non release modifying)

| | | | |
|---|---|---|---|
| 11 | Opadry Colored Coating System - PVA | 10.2 | 2.54 |
| | Based | | |
| | Total Tablet Weight | 401.2 | 100.0 |

Manufacturing Process: Dronabinol Loading onto Fujicalin (Adsorbate Formation)
A) Dissolve Dronabinol (1) in Ethanol (200 proof) in a suitable tank with a mixer.
B) Disperse Sodium Lauryl Sulfate (2) into (A). Continue to mix.
C) Charge Fujicalin (3) to a high shear granulator.
D) While mixing the Fujicalin, add the dispersion from (B). Mix until (B) is suitably dispersed.
E) Transfer the wet contents of the high shear granulator to a tray dryer.
F) Dry at 50 C (+/−10 C) to remove the Ethanol.
G) Collect the dried material from the trays and pass through a No. 20 mesh screen to deagglomerate.
H) Store in an HPDE drum double lined with PE bags and desiccants as required.

Blend for Compression
I) Charge the required quantity of the Dronabinol-Loaded Fujicalin (H) into a tumble blender after passing through a No. 20 mesh screen.
J) Charge the required quantity of Prosolv (4) and Hydroxypropyl cellulose (5) to the tumble blender after passing through a No. 20 mesh screen.
K) Close the blender and blend for 15 minutes.
L) Open the blender and charge the required quantity of Magnesium Stearate (6) after passing through a No. 20 mesh screen.
M) Close the blender and blend for 2 minutes.
N) Collect the Powder Blend (M) from the blender into an HDPE drum double lined with PE bags and desiccants as required.

Core Tablet Compression
O) Using a tablet press, compress tablet cores at a target tablet weight of 340 mg.
P) Collect the Dronabinol 10 mg Tablets into HDPE drums lined with PE bags and desiccants as required.

Controlled Release Coating (IR/DR Compartment)
Q) Prepare the required quantity of Ammonio Methacrylate Copolymer Coating Dispersion by adding the Triethyl Citrate to a mixture of Eudragit RL30D and Eudragit RL30D in a suitable tank with mixer.
R) Stir the Eudragit/Triethyl Citrate dispersion for not less than 30 minutes.
S) Disperse the required quantity of talc in sufficient purified water to provide a final solids content of the coating suspension at 15% w/w.
T) Combine the Eudragit/Triethyl Citrate dispersion and the talc/water dispersion. Mix the dispersion continuously.
U) Charge the required quantity of Dronabinol 10 mg tablet cores to a suitable tablet pan coater.
V) Apply a 15% weight gain of Ammonio Methacrylate Copolymer Coating Dispersion (T) to the tablet cores.
W) Allow the coated tablets to dry in the tablet coater for an additional period of time before applying the Outer Trade Dress Coating.

Tablet Coating (Non-release modifying trade dress)
X) Prepare the Opadry Coating System as an 18% w/w dispersion in water using a suitable tank and mixer.
Y) Apply a 3% weight gain of the Opadry Coating to the Controlled Release coated tablets (W).
Z) Collect the coated tablets from the tablet coater into HDPE drums lined with PE bags and desiccants as required.

Modifications.

With the teaching here, the skilled artisan can now modify this type of formulation to meet the target PK profile. For example, the following modification approaches are useful:
Manipulate the coating as set forth in Example 12
Alter properties of core tablet to provide for an osmotic

### Example 19

### Medicament: IR/DR (Monolithic/Matrix Tablet, Continuous)

A medicament is provided comprising a delayed release compartment and an immediate release compartment contained within the same dose unit. The IR and DR compartments are spatially oriented within a “continuous release” tablet as was also illustrated in Example 17 and Example 18.

The medicament provided in this Example 19 is a solid, co-precipitate type formulation where the co-precipitate facilitates solids handling of dronabinol and incorporation into a solid medicament (e.g. tablet).

TABLE 14
Monolithic/Matrix Tablet
10 mg Dronabinol (or other THC) Tablet

| | | | |
|---|---|---|---|
| No. | Ingredient | mg/tablet | % w/w |

Core Matrix Tablet (IR/DR Compartment)

| | | | |
|---|---|---|---|
| 1 | Dronabinol | 10.0 | 2.72 |
| 2 | Polyvinyl Caprolactam-Polyvinyl Acetate- | 90.0 | 24.46 |
| | Polyethylene Glycol Graft Copolymer | | |
| | (Soluplus) | | |
| 3 | Lactose Monohydrate | 70.0 | 19.03 |
| 4 | Microcrystalline Cellulose | 70.0 | 19.03 |
| 5 | Polyvinyl Acetate/Polyvinylpyrrolidone | 110.0 | 29.90 |
| | (Kollidon SR) | | |
| 6 | Colloidal Silicon Dioxide | 3.6 | 0.98 |
| 7 | Magnesium Stearate | 3.6 | 0.98 |
| | SubTotal | 357.2 | 97.1 |

Outer Trade Dress Coating (Non release modifying)

| | | | |
|---|---|---|---|
| 7 | Opadry Colored Coating System - PVA | 10.7 | 2.91 |
| | Based | | |
| | Total Tablet Weight | 367.9 | 100.0 |

Manufacturing Process: Spray Drying Dronabinol with SoluPlus (Coprecipitate Formation)
A) Dissolve Dronabinol (1) and Soluplus (2) in Ethanol (200 proof) in a suitable tank with a mixer.
B) Spray dry the dronabinol/Soluplus solution.
C) Collect the spray dried powder and dry in a vacuum oven at approximately 30 C to remove residual ethanol.

Roller Compaction
D) Blend Spray Dried Dronabinol (C) with Lactose Monohydrate (3), Microcrystalline Cellulose (4), and Kollidon SR (5) in a tumble mixer for 25 minutes.
E) Add one-half of the required quantity of each colloidal silicon dioxide (6) and magnesium stearate (7) and continue blending in the tumble mixer for 5 minutes.
F) Roller compact the powder blend (E) and pass the compacted ribbons through a suitable mill (i.e. Comil, Fitzmill, or oscillating granulator) to form granules.

Core Matrix Tablets (IR/DR Compartment)
G) Collect the milled granulate (F) and blend with the remaining quantity of colloidal silicon dioxide (6) and magnesium stearate (7) in a tumble mixer for 5 minutes.
H) Using a tablet press, compress tablet cores at a target tablet weight of 357.2 mg.
I) Collect the Dronabinol 10 mg Tablets into HDPE drums lined with PE bags and desiccants as required.

Tablet Coating (Non-release modifying trade dress)
J) Prepare the Opadry Coating System as an 18% w/w dispersion in water using a suitable tank and mixer.
K) Load the Dronabinol 10 mg Tablets (I) into a suitable tablet coater.
L) Apply a 3% weight gain of the Opadry Coating dispersion to the tablets.
M) Collect the coated tablets from the tablet coater into HDPE drums lined with PE bags and desiccants as required.

Modifications.

With the teaching here, the skilled artisan can now modify this type of formulation to meet the target PK profile. For example, the following modification approaches are useful:
Use different materials to prepare coprecipiate (e.g. sugar alcohols to make a glass, etc.)
Manipulate the matrix as set forth in Example 17
Use enteric polymers to make matrix tablets

### Example 20

### Medicament-Pellet Type, Continuous Release-Type

A medicament is provided comprising a delayed release compartment and an immediate release compartment contained within the same plurality of pellets in the dose unit. The IR and DR compartments are spatially oriented within a “continuous release” tablet as was also illustrated in Example 17, Example 18, and Example 19.

The medicament provided in this Example is a solid, coprecipitate to facilitate solids handling of dronabinol and incorporation into a plurality of pellets.

TABLE 15
Pellet in Capsules (IR/DR)
10 mg Dronabinol (or other THC) Capsule

| | | | |
|---|---|---|---|
| No. | Ingredient | mg/capsule | % w/w |

Core Matrix Pellets (IR/DR Compartment)

| | | | |
|---|---|---|---|
| 1 | Dronabinol | 10.0 | 4.00 |
| 2 | Cellulose Acetate Butyrate (CAB 381-0.5) | 177.5 | 71.00 |
| 3 | Triacetin | 12.5 | 5.00 |
| 4 | Low Substituted Hydroxypropyl Cellulose | 50.0 | 20.00 |
| | Target Fill Weight | 250.0 | 100.0 |

Manufacturing Process: Melt Extrusion with Cellulose Acetate Butyrate (Coprecipitate Formation)
A) Dissolve Dronabinol (1) and Triacetin (3) in Ethanol (200 proof) in a suitable tank with a mixer.
B) Using a melt extruder (ie twin screw with liquid feed addition port and vent station), prepare the dronabinol matrix pellets.
C) Meter the cellulose acetate butyrate (2) and low substituted hydroxypropyl cellulose (4) using loss in weight feeders into the extruder.
D) Meter the dronabinol/triacetin/ethanol solution into the extruder downstream of the powder feed and upstream of the vent station.
E) Extrude molten mixture using heat and screw shear through a rod die.
F) Process the cylindrical extrudate into pellets using a rotary cutter.
G) Collect the pellets.
H) Using a suitable encapsulator, fill suitably sized two piece hard capsule shells with 250 mg of melt extruded pellets.

Modifications.

With the teaching here, the skilled artisan can now modify this type of formulation to meet the target PK profile. For example, the following modification approaches are useful:
Adjust ratio of drug to excipients
Adjust ratio of non-rate controlling excipients to rate controlling excipients
Use different polymers or polymer grades
Use different processing aids (e.g. different plasticizers if needed)
Use different fillers to manipulate release
Vary pellet size to modify release (smaller pellets typically release faster)
Can compress pellets into a tablet to create a singular dosage unit like in Example 17 and Example 19

### Example 21

### Medicament: Monolithic/Matrix Tablet, Continuous

This is another example of the present medicaments of the monolithic/matrix-type. The dosage unit is designed to swell in the stomach after ingestion and be retained for sufficient quantity of time while continuously releasing the dose. This is another example where the IR and DR compartments can be defined spatially. This is also another example of a useful “solid adsorbate” type dosage form.

TABLE 16
Monolithic/Matrix Tablet (IR/DR) - Gastroretentive
10 mg Dronabinol (or other THC) Tablet

| | | | |
|---|---|---|---|
| No. | Ingredient | mg/tablet | % w/w |

Core Matrix Tablet (IR/DR Compartment)

| | | | |
|---|---|---|---|
| 1 | Dronabinol | 10.0 | 1.63 |
| 2 | Sodium Lauryl Sulfate | 10.0 | 1.63 |
| 3 | Fujicalin (Dicalcium Phosphate Anhydrous) | 200.0 | 32.68 |
| 4 | Polyethylene Oxide (Polyox N-60K) | 70.0 | 11.44 |
| 5 | Polyethylene Oxide (Polyox N-80) | 304.0 | 49.67 |
| 6 | Magnesium Stearate | 6.0 | 0.98 |
| | SubTotal | 600.0 | 98.0 |

Outer Trade Dress Coating (Non release modifying)

| | | | |
|---|---|---|---|
| 7 | Opadry Colored Coating System - PVA Based | 12.0 | 1.96 |
| | Total Tablet Weight | 612.0 | 100.0 |

Manufacturing Process: Dronabinol Loading onto Fujicalin (Adsorbate Formation)
A) Dissolve Dronabinol (1) in Ethanol (200 proof) in a suitable tank with a mixer.
B) Disperse Sodium Lauryl Sulfate (2) into (A). Continue to mix.
C) Charge Fujicalin (3) to a high shear granulator.
D) While mixing the Fujicalin, add the dispersion from (B). Mix until (B) is suitably dispersed.
E) Transfer the wet contents of the high shear granulator to a tray dryer.
F) Dry at 50 C (+/−10 C) to remove the Ethanol.
G) Collect the dried material from the trays and pass through a No. 20 mesh screen to deagglomerate.
H) Store in an HPDE drum double lined with PE bags and desiccants as required.

Core Matrix Tablets (IR/DR Compartment)
I) Charge the required quantity of the Dronabinol-Loaded Fujicalin (H) into a tumble blender after passing through a No. 20 mesh screen.
J) Charge the required quantity of Polyox N-60K (4) and Polyox N-80 (5) to the tumble blender after passing through a No. 20 mesh screen.
K) Close the blender and blend for 15 minutes.
L) Open the blender and charge the required quantity of Magnesium Stearate (6) after passing through a No. 20 mesh screen.
M) Close the blender and blend for 2 minutes.
N) Collect the Powder Blend (M) from the blender into an HDPE drum double lined with PE bags and desiccants as required.

Tablet Compression
O) Using a tablet press, compress tablet cores at a target tablet weight of 600 mg.
P) Collect the Dronabinol 10 mg Tablets into HDPE drums lined with PE bags and desiccants as required.

Tablet Coating (Non release modifying)
Q) Prepare the Opadry Coating System as an 18% w/w dispersion in water using a suitable tank and mixer.
R) Load the Dronabinol 10 mg Tablets (P) into a suitable tablet coater.
S) Apply a 3% weight gain of the Opadry Coating dispersion to the tablets.
T) Collect the coated tablets from the tablet coater into HDPE drums lined with PE bags and desiccants as required.

Modifications.

With the teaching here, the skilled artisan can now modify this type of formulation to meet the target PK profile. For example, the following modification approaches are useful:
See adjustments to matrix tablets as set forth in Example 17
Add materials to make dosage form float (i.e. disintegrants, effervescent salts)
Adjust tablet size and shape

### Example 22

### Medicament: Monolithic/Matrix Tablet, Continuous Release

This is another example of a monolithic/matrix-type tablet of the present invention.

TABLE 17
Monolithic/Matrix Tablet (IR/DR)
10 mg Dronabinol (or other THC) Tablet

| | | | |
|---|---|---|---|
| No. | Ingredient | mg/tablet | % w/w |

Core Matrix Tablet (IR/DR Compartment)

| | | | |
|---|---|---|---|
| 1 | Dronabinol | 10.0 | 2.27 |
| 2 | Hydroxypropyl Beta Cyclodextrin | 150.0 | 33.98 |
| 3 | Lactose Monohydrate | 75.0 | 16.99 |
| 4 | Microcrystalline Cellulose | 75.0 | 16.99 |
| 5 | Hypromellose (Methocel K100M Premium | 110.0 | 24.92 |
| | DC) | | |
| 6 | Colloidal Silicon Dioxide | 4.3 | 0.97 |
| 7 | Magnesium Stearate | 4.3 | 0.97 |
| | SubTotal | 428.6 | 97.1 |

Outer Trade Dress Coating (Non release modifying)

| | | | |
|---|---|---|---|
| 8 | Opadry Colored Coating System - PVA | 12.9 | 2.92 |
| | Based | | |
| | Total Tablet Weight | 441.5 | 100.0 |

Manufacturing Process: Spray Drying Dronabinol with Cyclodextrin (Complex Formation)
A) Dissolve Dronabinol (1) and Hydroxypropyl Beta Cyclodextrin (2) in Ethanol (200 proof) in a suitable tank with a mixer.
B) Spray dry the dronabinol/cyclodextrin solution.
C) Collect the spray dried powder and dry in a vacuum oven at approximately 30 C to remove residual ethanol.

Roller Compaction
D) Blend Spray Dried Dronabinol (C) with Lactose Monohydrate (3), Microcrystalline Cellulose (4), and Methocel (5) in a tumble mixer for 25 minutes.
E) Add one-half of the required quantity of each colloidal silicon dioxide (6) and magnesium stearate (7) and continue blending in the tumble mixer for 5 minutes.
F) Roller compact the powder blend (E) and pass the compacted ribbons through a suitable mill (i.e. Comil, Fitzmill, or oscillating granulator) to form granules.

Core Matrix Tablets (IR/DR Compartment)
G) Collect the milled granulate (F) and blend with the remaining quantity of colloidal silicon dioxide (6) and magnesium stearate (7) in a tumble mixer for 5 minutes.
H) Using a tablet press, compress tablet cores at a target tablet weight of 428.6 mg.
I) Collect the Dronabinol 10 mg Tablets into HDPE drums lined with PE bags and desiccants as required.

Tablet Coating (Non release modifying)
J) Prepare the Opadry Coating System as an 18% w/w dispersion in water using a suitable tank and mixer.
K) Load the Dronabinol 10 mg Tablets (I) into a suitable tablet coater.
L) Apply a 3% weight gain of the Opadry Coating dispersion to the tablets.
M) Collect the coated tablets from the tablet coater into HDPE drums lined with PE bags and desiccants as required.

Modifications.

With the teaching here, the skilled artisan can now modify this type of formulation to meet the target PK profile. For example, the following modification approaches are useful:
Adjust complexation agent (e.g. different substitutions and types of cyclodextrins)
Adjust the matrix tablet as set forth in Example 17.

### Example 23

### PK Profile Tailoring

The present invention provides medicaments which produce superior PK profiles. The medicaments of the present invention can be produced using a wide variety of formulation types and manufacturing processes and are not limited to any specific recipe. With the teachings provided herein, the skilled artisan can now, without undue experimentation, produce medicaments which produce a desired PK profile (e.g. any of PK profiles 1-19). For example, a medicament of the present invention can be produced by:
1) Produce one or more medicaments according to the present invention which, based upon the teaching here, is predicted to provide the target (desired) PK profile (the “test” medicaments”).
2) Perform an in vitro dissolution test on the test medicaments and an immediate release cannabinoid medicament (the “comparator”) with a known PK profile, such as a Marinol capsule (e.g. as detailed in Example 10);
3) Based upon the results of the in vitro dissolution assay of the test medicament and the comparator medicament, predict the pK of the test medicament using pK modeling as is generally well known by the skilled artisan.
4) Compare the target PK profile with the predicted test medicament PK profile.
5) Based upon any deviation between the target profile and the predicted (modeled) test medicament PK profile, modify the test medicament according to the teachings here to better approximate the target PK profile (the final test medicament).
6) Perform the in vitro dissolution test of step (2), the PK modeling of step (3), and the comparison of step (4). Repeat as necessary to obtain a predicted (modeled) pK profile that approximates the target pK profile.
7) Perform an in vivo PK analysis of the final test medicament on a study population that is representative of the target patient population. When the target patient population is sleep apnea, the medicament can be administered during a given window before bed (e.g. 30 mins), for example, as detailed in Example 1. Blood samples can be obtained at regular intervals to measure cannabinoid and/or metabolite levels throughout the window of the test subject's sleep without waking the test subject (using, e.g. a heparin lock cannula).
8) Compare the observed in vivo PK profile to the target PK profile. Modify the final test medicament as necessary by repeating the above steps.

We claim:

1. A medicament comprising a cannabinoid partitioned in an immediate release compartment and in a delayed release compartment wherein the cannabinoid is present in an amount of about 1 to about 75 mg and optionally wherein when administered to a human subject, the medicament provides a PK Profile of any one of PK Profiles 1-20.

2. The medicament of claim 1 wherein about 25% to about 90% of the cannabinoid is in the immediate release dosage compartment, optionally wherein about 25% to about 75% of the cannabinoid is in the immediate release dosage compartment.

3. The medicament of claim 2 wherein the delayed release compartment is a solid dosage compartment.

4. The medicament of claim 3 wherein the solid dosage compartment is a lipid dosage compartment, a coprecipiate compartment, an adsorbate compartment, a sugar glass compartment, a crystalline cannabinoid, a solubilizing compartment, or an osmotic compartment.

5. The medicament of claim 3 wherein the solid dosage compartment is an osmotic compartment comprising a core comprising the cannabinoid and an expandable polymer; a semipermeable membrane surrounding the core; and a passageway disposed in the semipermeable membrane.

6. The medicament of claim 2 wherein the delayed release dosage compartment is a semi-solid dosage compartment.

7. The medicament of claim 6 wherein the semi-solid dosage compartment is a SEDDS dosage compartment.

8. The medicament of claim 7 wherein the SEDDS dosage compartment comprises an oily medium, at least one surfactant, and a semi-solid inducer.

9. The medicament of claim 1 wherein the immediate release dosage compartment is a liquid immediate release compartment

10. The medicament of claim 9 wherein the liquid immediate release compartment comprises a lipophilic medium or oil.

11. The medicament of claim 10 wherein the lipophilic medium or oil is cottonseed oil, sesame oil, coconut oil or peanut oil.

12. The medicament of claim 1 wherein the delayed release compartment is a liquid compartment, a solid compartment, or a semi-solid compartment and wherein the delayed release compartment comprises a delayed release coating.

13. The medicament of claim 12 wherein the delayed release coating is a cellulosic polymer or an acrylic polymer.

14. The medicament of claim 1 wherein the delayed release compartment is a liquid or semi-solid compartment in the form of a soft-gel, a hard-gel, or a plurality of microparticles.

15. The medicament of claim 1 wherein the delayed release compartment is a solid dosage compartment in the form of a tablet or a plurality of microparticles.

16. The medicament of dosage form of claim 1 wherein the delayed release compartment is any one of the second dosage compartments of Table 1.

17. The medicament of claim 1 wherein, the cannabinoid is dronabinol.

18. The medicament of claim 1 wherein the cannabinoid is in an amount of about 0.5 to about 25 mg.

19. A method of treating a cannabinoid-sensitive disorder comprising administering to a subject in need thereof the medicament of claim 17.

20. A method of establishing an optimal dose of a medicament for a subject with a cannabinoid-sensitive disorder comprising the steps of:
(a) administering a first dose of the medicament for a first treatment period;
(b) administering a second dose of the medicament for a second treatment period wherein the second dose is increased compared to the first dose;
(c) optionally administering a third dose of the medicament for a third treatment period wherein the third dose is increased compared to the second dose;
(d) if step c is performed, optionally administering a fourth dose of the medicament for a fourth treatment period wherein the fourth dose is increased compared to the third dose;
(e) if step d is performed, optionally administering a fifth dose of the medicament for a fifth treatment period wherein the fifth dose is increased compared to the fourth dose;
(f) if step e is performed, optionally administering a sixth dose of the medicament for a sixth treatment period wherein the sixth dose is increased compared to the fifth dose; and
(g) if step f is performed, optionally administering a sixth dose of the medicament for a sixth treatment period wherein the sixth dose is increased compared to the fifth dose,
wherein the medicament is a medicament of claim 1.
